(19) United States
(12) Patent Application Publication
(10) Pub. No.: US 2004/0038222 A1
(43) Pub. Date: Feb. 26, 2004

(54) Anthrax susceptibility gene

(76) Inventors: William F. Dietrich, Newton, MA (US); James W. Watters, St. Louis, MO (US)

Correspondence Address: EDWARDS & ANGELL, LLP, P.O. BOX 9169, BOSTON, MA, 02209, US

(21) Appl. No.: 10/261,175

(22) Filed: Sep. 30, 2002

Related U.S. Application Data
(60) Provisional application No. 60/325,864, filed on Sep. 29, 2001.

(51) Int. Cl. C12Q 1/68; C07H 21/04; C12N 9/12; C12P 21/02; C12N 5/06

(52) U.S. Cl. 435/6; 435/69.1; 435/194; 435/320.1; 435/325; 536/23.2

(57) ABSTRACT

The Ltxsl gene has been cloned as KiflC. KiflC encodes a kinesin-like motor protein of the UNC104 subfamily. Nucleic acid sequences of KiflC are also disclosed that confer LeTx resistance to cells, particularly mammalian cells such as macrophages. Therapeutic methods are provided to treat a subject susceptible to anthrax.

The present application claims the benefit of U.S. provisional application number 60/325,864, filed Sep. 29, 2001, which is incorporated by reference herein in it entirety.

This invention was supported in part by a grant from the National Institutes of Health (AI 43321) and by the Howard Hughes Medical Institute. The U.S. Government has certain rights in this invention.

### BACKGROUND OF THE INVENTION

1. Field of the Invention

The invention relates to inter alia an anthrax susceptible gene and diagnostic and therapeutic uses thereof.

2. Background

Anthrax infection is mediated by spores of Bacillus anthracis, which can gain entry to the body through breaks in the skin, through inhalation, or through ingestion. Fatal anthrax is characterized by the establishment of a systemic bacteremia that is accompanied by an overwhelming toxemia. It seems that anthrax is a 2-pronged attack with the bacteremia and/or toxemia contributing to the fatal syndrome of shock, hypoperfusion, and multiple organ system failure. The likelihood of developing systemic disease varies with the portal of organism entry, and is most pronounced for the inhalational route (reviewed in Dixon et al., 1999, New England J. Med. 341: 815-826).

Diagnosis, treatment, and prophylaxis for systemic anthrax are difficult. The initial symptoms of systemic (especially inhalation) anthrax can be rather non-specific, forcing the clinician to consider conflicting diagnostic possibilities (Dixon et al, 1999, supra). Even though B. anthracis is sensitive to many antibiotics, successful treatment of systemic anthrax depends on rapid diagnosis and immediate, aggressive intervention (Dixon et al., 1999, supra). While vaccination has long been an important means for controlling anthrax in livestock and in at-risk humans, the low incidence of naturally occurring human anthrax has, until now, made it difficult to justify a population-wide vaccination campaign; however, while clinical anthrax is rare, there is growing concern over the potential use of B. anthracis in biological warfare and terrorism.

B. anthracis possesses two main virulence factors, a poly-D-glutamic capsule and a tripartite protein toxin. Anthrax toxin is responsible for the major symptoms of the disease; however, as with any infectious disease, there are many interactions between B. anthracis and its host that help to determine the infection outcome. One of the most important host-pathogen interactions in anthrax involves the response of the host to two bacterially produced toxin activities, collectively referred to as anthrax toxin. Well-established lines of evidence indicate that anthrax toxin is a crucial part of the pathogenesis of anthrax (reviewed in Leppla, 1991, “The anthrax toxin complex.” pp. 277-302 in Sourcebook of Bacterial Protein Toxins., J. Alouf, ed., Academic Press, New York, N.Y.). First, B. anthracis strains that lack the ability to deliver toxin into cells are essentially avirulent. Second, the basis of all current vaccinations against anthrax require the induction of protective immunity against toxin components. Third, administration of bacterium-free toxin preparations to animal models can induce death in those animals in a manner reminiscent of the death seen in systemic anthrax patients. Fourth, anthrax-infected guinea pigs can be rescued from fatality through the administration of antibiotics, but only before the bacteria reach a titer of greater than 10⁶ bacteria/ml in the bloodstream; after that point, antibiotic therapy is useless, suggesting that the buildup of toxin in the bloodstream has reached the point of no return.

Anthrax toxin consists of three proteins that comprise two distinct “A-B” toxin activities (Leppla, 1991, supra). The proteins, called Edema Factor (EF) and Lethal Factor (LF), are different catalytic “A” domains that each utilize the same cell-binding “B” moiety, called Protective Antigen (PA), to gain access to the cytosol of cells. The combination of PA and EF are referred to as Edema Toxin (EdTx). The combination of PA and LF are called Lethal Toxin (LeTx). A great deal is now known about the mechanisms of cellular entry of these toxins (Friedlander, 1986, J. Biol. Chem. 261: 7123-7126; Klimpel et al., 1992, Proc. Natl. Acad. Sci. U.S.A. 89: 10277-10281; Menard et al., 1996, FEBS Letters 386: 161-164; Gordon et al., 1997, Infect. Immun. 65: 3370-3375; Petosa et al., 1997, Nature 385: 833-838; Beauregard et al., 2000, Cell. Microbiol. 2: 251-258; Sellman et al., 2001, J. Biol. Chem. 276: 8371-8376; Sellman et al., 2001, Scienc,e 292: 695-697. However, the roles that these toxins play in the establishment of infection and the development of disease are incompletely understood.

PA, the non-toxic, cell-binding component of the toxin, is the essential component of the currently available human vaccine. The vaccine is usually produced from batch cultures of the Sterne strain of B. anthracis, which although avirulent, is still required to be handled as a Class III pathogen. In addition to PA, the vaccine contains small amounts of the anthrax toxin moieties, EF and LF, and a range of culture derived proteins. All these factors contribute to the recorded reactogenicity of the vaccine in some individuals. The vaccine is expensive and requires a six month course of four vaccinations. Furthermore, present evidence suggests that this vaccine may not be effective against inhalation challenge with certain strains (Broster et al., 1990, Proceedings of the International Workshop on Anthrax, Apr. 11-13, 1989, Winchester UK. Salisbury Med. Bull. Suppl. No. 68, pp. 91-92).

Although promptly-administered antibiotics may eradicate the bacteria, the harmful effects of the infection persist because of the continuing action of the toxin. Thus, antibiotic therapy is of limited efficacy once symptoms have become evident. Antibiotic-resistant strains that continue to emerge further exacerbate attempts at the treatment of anthrax infection. There is thus, a need for alternative forms of therapy or therapy that can be used adjunct to antibiotic therapy, such as identification of molecules that render mammals of certain haplotypes resistant to the effects of LeTx. Identification of the genes producing these proteins will aid in the development of specific inhibitors of the action of toxin.

For these reasons, it is desirable to identify individuals who may not require vaccination and also to devise novel therapeutic strategies.

The symptoms of lethal anthrax infection can be mimicked in animal models by the administration of anthrax LeTx, which is produced at high levels during systemic infection (Welkos et al., 1986, Infect. Immun. 51: 795-800). While PA mediates the intracellular delivery of LF into the cytosol of all cell types that have been tested, LF exerts a cytolytic effect that is specific to macrophages (Leppla, 1991, supra; Petosa et al., 1997, Nature, 385: 833-838). One therapeutic strategy would be to prevent the transport of LF to its intracellular destination, and thereby prevent cytolysis.

LeTx has been suggested to play important roles in both the early and late stages of anthrax infection. For example, there is controversial data suggesting that early in infection, sublytic amounts of LeTx can affect the production of several macrophage inflammatory signals (Hanna et al., 1993, Proc. Natl. Acad. Sci. U.S.A. 90: 10198-10201; Pellizari et al., 1999, FEBS Letters, 462: 199-204; Shin et al., 2000, Cell Biol. and Toxicol. 16: 165-174; Erwin et al., 2001, Infect. Immun. 69: 1175-1177). In contrast, larger amounts of LeTx (which mimic the levels of toxin in the bloodstream of a individual with end-stage systemic anthrax infection) can induce fatal shock when administered i.v. to animal models (reviewed in Leppla, 1991, supra), and cause the rapid cytolysis of macrophages (and apparently no other cell type) in vitro (Friedlander, 1986, J. Biol. Chem. 261: 7123-7126).

LF has a zinc metallopeptidase activity that is required for its cellular toxicity (Klimpel et al., 1994, Mol. Microbiol., 13: 1093-1100). Interestingly, it has been observed that LF can physically interact with and cleave the N-terminal tails from a number of different Map-Kinase-Kinase (MAPKK) proteins, likely altering their signalling functions (Duesbery et al., 1998, Science, 280: 734-737; Vitale, et al., 2000, Biochem. J., 352: 739-745; Pellizzari et al., 1999, FEBS Letters, 462: 199-204; Vitale et al., 1998, Biochem. Biophys. Res. Comm. 248: 706-711).

The cytolysis of macrophages that is induced by LF intoxication more closely resembles necrosis than it does apoptosis, and is perhaps a response to overproduction of- or inappropriately contained exposure to cytokines or reactive oxygen intermediates (Lin et al., 1996, Curr. Microbiol., 33: 224-227). The toxic activity of LF depends on its Zn²⁺ metallopeptidase activity (Klimpel et al., 1994, Mol. Microbiol., 13: 1093-1100; Hammond and Hanna, 1998, Infect. Immun. 66: 2374-2378), strongly suggesting that proteolysis of one or more cellular proteins unleashes a cascade of events that results in the death of the intoxicated macrophage. In support of this model, LF cleaves several distinct mitogen-activated protein kinase kinase (MAPKK) species; however, the physiological importance of this MAPKK proteolysis relative to macrophage cytolysis has not been established (Duesbery et al., 1998, Science, 280: 734-737; Vitale et al., 2000, Biochem. J., 352 pt. 3: 739-745).

Given the importance of the macrophage in the host response to infection and replication of B. anthracis, many investigations have focused on understanding the macrophage-specific cytolytic effects of LeTx. A number of physiologic changes have been noted in intoxicated macrophages prior to their lysis, including alterations in membrane permeability, conversion of ATP stores into ADP and AMP, and cessation of macromolecular synthesis (Hanna et al., 1992, Mol. Bio. Cell., 3: 1269-1277). There also appears to be a requirement for protein synthesis and for the activity of the proteasome in order for the cytolytic effect to occur (Bhatnagar and Friedlander, 1994, Infect. Immun., 62:2958-2962; Tang and Leppla, 1999, Infect. Immun., 67: 3055-3060). In addition, connections have been made between sensitivity to LeTx and the ability to mount an oxidative burst; compounds that act as antioxidants provide protection of the macrophage from cytolysis (Hanna et al., 1994, Mol. Med., 1: 7-18). The invention described hereinbelow provides methods for the identification of key elements of the LeTx-mediated cytolytic pathway, which may be advantageously used as drug targets.

It would be desirable to develop drugs or inhibitory molecules to render susceptible mammals resistant to the lethal effects of LeTx.

The manifestation of anthrax is a complex process, and the outcome of anthrax infection depends on many complex host-pathogen interactions (Welkos et al., 1996, supra; Leppla, 1991, supra; Welkos and Friedlander, 1988, Microb. Pathol., 4: 53-69). Because LeTx-induced cytolysis of macrophages plays an important role in the overall outcome of anthrax infection, the identification of a gene that affects macrophage susceptibility to LeTx would provide valuable insight into a poorly understood aspect of anthrax pathogenesis and enable the development of therapeutics aimed at blocking lethal intoxication of cells during anthrax infection.

The relative scarcity of well-described human anthrax cases has hampered the epidemiologic definition of risk factors. Spore exposure and age are two major risk factors identified thus far (Abramova et al., 1993, Proc. Natl. Acad. Sci. U.S.A. 90: 2291-2294; Meselson et al., 1994, Science, 266: 1202-1208). The invention provides methods for identifying genetic factors such as may be responsible for unexplained human variation in susceptibility to anthrax spore exposure that have been reported (Dahlgren et al., 1960, Am. J. Hyg., 72:24-31; Albrink et al., 1960, Am. J. Pathol., 36: 457-471; Brachman et al., 1966, Bacteriol. Rev., 30: 646-659).

It has been noted for several years that cultured macrophages from different inbred mouse strains exhibit differences in susceptibility to cytolysis caused by LeTx (Singh et al., 1989, J. Biol. Chem., 264:11099-11102; Friedlander et al., 1993, Infect. Immun., 61: 245-252). More recently, we have extended these observations, noting that sensitivity and resistance to LeTx is a common polymorphism among laboratory mouse stocks, and that, in many cases, the inheritance of this phenotype difference is due to a locus called Ltxsl on chromosome 11 (Roberts et al., 1998, Mol. Microbiol., 29: 581-591; Watters and Dietrich, 2001, Genomics, 73: 223-231). Human variation in macrophage susceptibility to anthrax lethal toxin has been reported (Hanna et al., 1994, Mol. Med., 1: 7-18). The invention provides methods for the identification of genes linked to anthrax susceptibility or resistance, and allelic variants of such genes that mediate the susceptible or resistant phenotype.

### SUMMARY OF THE INVENTION

The invention provides methods for the identification of genes and variants or variants thereof that can render macrophages resistant to intoxication with anthrax lethal factor. More particularly, the invention provides kinesin sequences that are involved in intracellular transportation of molecules. The KiflC molecular motor is shown herein to be an important mediator of macrophage resistance to intoxication with anthrax lethal factor. It is further shown that cleavage of map kinase kinase 3, a target of LeTx proteolysis, occurs in resistant cells.

Nucleic acid sequences of allelic variants of KiflC are also disclosed that confer LeTx resistance to cells, particularly mammalian cells such as macrophages. Critical amino acid substitutions involved in the resistance to LeTx are identified.

The invention provides methods for the identification of more such allelic variants.

The invention also encompasses methods for the identification of additional genes that mediate sensitivity or resistance to anthrax infection.

Currently-available anthrax vaccines may be accompanied by potentially severe side effects; therefore, it is desirable to identify individuals who are relatively resistant to anthrax infection in order to avoid needless vaccination of persons who are at low risk of disease. The invention provides methods of determining the susceptibility or resistance of an individual to anthrax infection, which methods may be used either in research or in diagnostic screening. Kits and reagents for use in such methods additionally are provided.

The invention also encompasses methods for the identification of novel therapeutic and/or prophylactic compositions directed at the treatment or prevention of anthrax infection.

Vectors also are provided that comprise nucleic acid sequences that code for KiflC that confer that confer LeTx resistance to cells, such as macrophages, are provided for. These vectors may be used as treatment to protect individuals susceptible to, or suffering from the lethal effects of LeTx.

A preferred use of nucleic acid sequences (anthrax resistance genes) of the invention is to generate therapeutic agents that can ameliorate the toxic effects of LeTx. The anthrax resistant genes can be expressed by a vector containing a DNA segment encoding the wild-type, alleles, variants, mutations or fragments of the genes. Mutations and variants, of the anthrax resistance genes are also preferably used in the construction of a vector. The vector comprising the desired nucleic acid sequence for conferring resistance to LeTx, preferably has at least one such nucleic acid sequence. Alternatively, the vector may be comprised of more than one such nucleic acid sequence, or combinations of allelic variants. The vector can also be comprised of cassettes of different allelic variants or wild type LeTx resistance genes.

Therapeutic methods of the invention include use of a nucleic acid sequence or amino acid sequence as disclosed herein to treat a subject in need of such treatment, particularly a mammal such as a primate especially a human suffering from or susceptible to adverse effects of LeTx. In a preferred approach, a nucleic acid sequence or amino acid sequence is employed to protect a subject against adverse effects of LeTx, i.e. to treat a subject against anthrax. Preferred administered agents include the entire nucleic acid sequence of the KiflC molecule, variants and fragments thereof; modified nucleic acid sequences of the KiflC molecule; and the entire amino acid sequence of the KiflC molecule or fragments thereof; modified amino acid fragments of the KiflC molecule.

Pharmaceutical compositions also are provided that include a nucleic acid or amino acid sequence as disclosed optionally admixed together with a pharmaceutically acceptable carrier.

In an especially preferred aspect of the invention, KiflC, variants, fragments, or oligopeptides thereof are used for identifying agents which may be useful for a number of therapeutic applications, including to reduce LeX effects. In particular, KiflC, variants, fragments, or oligopeptides thereof can be employed to identify such candidate agents, such as by determining which compounds bind or otherwise interact with such KiflC, variants, fragments, or oligopeptides thereof and for determining potential therapeutic agents, agents which compete for binding, or inhibitors which prevent candidate agents from binding.

The invention also includes methods to identify component(s) of a test sample by contacting a test sample with the KiflC gene, an allele or fragment thereof, or expression product of the KiflC gene, an allele or fragment thereof; and detecting interaction of the test sample with the KiflC gene, an allele or fragment thereof, or expression product of the KiflC gene, an allele or fragment thereof. Suitable test samples may include mammalian tissue sample, or a fluid sample such as mammalian plasma, saliva or urine sample.

The invention also includes methods for identifying one or more genes that can ate anthrax susceptibility in a mammal, which may comprise hridizing an isolated nucleic acid sequence with a KiflC nucleic acid probe to form a hybridized molecule, and detecting (or identifying) sequences hybridized to the probe. Preferably, nucleic acid sequences hybridized to the probe are compared to genomic synteny sequences from a gene database and then suitably mapped to locations on a syntenic chromosome based on percent homologies. Preferably, sequences hybridized to the probe are at least about 80 percent homologous, more preferably at least about 80, 90 or 95 percent homologous to mammalian chromosomal syntenic sequences. Expression products of the identified nucleic acid sequences also are suitably evaluated.

The invention also includes use of nucleic acid sequences and variants thereof, that render susceptible macrophages, from anthrax toxin susceptible mouse strains, resistant to the toxic effects of the anthrax toxin.

Preferred nucleic acid sequence code for a modified susceptible KiflC amino acid sequence. Specifically preferred sequences include those set forth in FIGS. 6A-6U. A “modified” susceptible KiflC amino acid sequence can be provided by a mutated KiflC gene, and particularly a “modified” susceptible KiflC amino acid sequence has one or more amino acids that differ from the amino acid sequence encoded by the non-mutated KiflC gene and/or at least one amino acid substitution or deletion that is capable of conferring resistance to adverse effects of LeTx.

Particularly preferred modified susceptible KiflC amino acid sequences will exhibit discernable functional differences from a non-modified sequence, e.g. a discernable (e.g. 10% difference) in a standard viability assay as defined herein.

Also preferably, a modified susceptible KiflC amino acid sequence contains some type of motif (i.e. epitope) which can reduce adverse effects of LeTx, e.g. as may be determined by an in vitro or in vivo assay.

An “allele” or “variant” is an alternative form of a gene. Of particular utility in the invention are variants of the gene encoding KiflC. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence.

Preferred KiflC nucleic acid sequences of the invention are shown in FIGS. 6A through 6U (SEQ ID NOS: 1 through 23). In accordance with the invention, any nucleic acid sequence that shows at least about a 50% sequence identity (homology) to any of the disclosed sequences and results in an increased viability of cells, or resistance to the lethal effects of to LeTx are preferred, more preferably these sequences have at least about 70 percent sequence identity, still more preferably at least about 80, 85, 90, 95, or 98 percent sequence identity to one or more of the sequences shown in FIGS. 6A through 6U. For purposes of determining sequence identity, a BLAST program is employed. Intron regions are typically excluded in determining sequence identity percents.

As referred to herein, functional fragments of a nucleic acid sequence indicate that the fragment will exhibit some type of activity in a standard viability assay, particularly at least about a 10 percent reduction in cellular death of treated cells as determined by a standard viability assay as that assay as defined herein (dye assay), more preferably at least about a 20, 30, 40 or 50 percent reduction in cellular death of treated cells as determined by a standard viability assay. Typical nucleic acid fragments comprise at least about 10 or 15 nucleic acid residues (nucleotides), more preferably at least about 20, 30, 40, 50, 60, 70, 80, 90, 100, 150 or 200 nucleic acid residues.

“Altered” nucleic acid sequences encoding KiflC include those sequences with deletions, insertions, or substitutions of different nucleotides, resulting in a polypeptide the same as KiflC or a polypeptide with at least one functional characteristic of KiflC. Included within this definition are polymorphisms which may or may not be readily detectable using a particular oligonucleotide probe of the polynucleotide encoding KiflC, and improper or unexpected hybridization to allelic variants, with a locus other than the normal chromosomal locus for the polynucleotide sequence encoding KiflC. The encoded protein may also be “altered,” and may contain deletions, insertions, or substitutions of amino acid residues which produce a silent change and result in a functionally equivalent KiflC. Deliberate amino acid substitutions may be made on the basis of similarity in polarity, charge, solubility, hydrophobicity, hydrophilicity, and/or the amphipathic nature of the residues, as long as the biological activity of KiflC is retained. For example, negatively charged amino acids may include aspartic acid and glutamic acid, positively charged amino acids may include lysine and arginine, and amino acids with uncharged polar head groups having hydrophilicity values may include leucine, isoleucine, and valine; glycine and alanine; asparagine and glutamine; serine and threonine; and phenylalanine and tyrosine.

The terms “amino acid” or “amino acid sequence” refer to an oligopeptide, peptide, polypeptide, or protein sequence, or a fragment of any of these, and to naturally occurring or synthetic molecules. In this context, “fragments,” refer to fragments of KiflC which are preferably at least or 10 to about 30 or 50, 60, 70, 80 90 or 100 amino acids in length, more preferably at least 15, 20, 25, 30, 40, or 50 amino acids, and preferably which retain some biological activity of KiflC. Where “amino acid sequence” is recited to refer to an amino acid sequence of a naturally occurring protein molecule, “amino acid sequence” and like terms are not meant to limit the amino acid sequence to the complete native amino acid sequence associated with the recited protein molecule.

“Amplification” relates to the production of additional copies of a nucleic acid sequence. Amplification is generally carried out using polymerase chain reaction (PCR) technologies or by isothermal amplification technologies well known in the art.

As used herein, the term “genetic construct” is meant to refer to plasmids which comprise anthrax lethal factor resistant coding sequences that encode an anthrax lethal factor resistant protein and an amino acid sequence that directs intracellular protein trafficking, the coding sequences being operably linked to regulatory elements required for expression of the coding sequences in eukaryotic cells. Regulatory elements for DNA expression include a promoter and a polyadenylation signal. In addition, other elements, such as a Kozak region, may also be included in the genetic construct. Initiation and termination signals are required regulatory elements which are often considered part of the coding sequence. The coding sequences of genetic constructs of the invention include functional initiation and termination signals.

As used herein, “gene” means a nucleotide sequence that contains a complete coding sequence. Generally, “genes” also include nucleotide sequences found upstream (e.g. promoter sequences, enhancers, etc.) or downstream (e.g. transcription termination signals, polyadenylation sites, etc.) of the coding sequence that affect the expression of the encoded polypeptide.

As used herein, a “gene product” is either a DNA or RNA (mRNA) copy of a portion of a gene, or a corresponding amino acid sequence translated from mRNA.

As used herein, the term “protective”, when applied to nucleic acids and proteins of the present invention, means a version of a nucleic acid or protein that functions in a manner to confer cellular resistance to the lethal effects of LeTx. Protective variants or other variants of a given nucleic acid or protein may function either normally or abnormally with respect to functions that do not relate to anthrax susceptibility.

As used herein, the term “non-protective” when applied to nucleic acids and proteins of the present invention, means an allele or other variant of a nucleic acid or protein that renders, whether through loss or gain of function, the bearer susceptible to lethal effects of LeTx. Non-protective variants or other variants of a nucleic acid or polypeptide of the invention may differ structurally from their protective counterparts in any of a variety of ways including, but not limited to, differences in the amino acid sequence of an encoded polypeptide and/or differences in expression levels of an encoded nucleotide transcript of polypeptide product. Such variants may function either normally or abnormally with respect to functions that do not relate to anthrax susceptibility.

For example, the nucleotide sequence of a non-protective allele of a nucleic acid of the invention may differ from that of a protective allele by, for example, addition, deletion, substitution, and/or rearrangement of nucleotides.

As used herein, the terms “upstream” and “downstream” are art-understood terms referring to the position of an element of nucleotide sequence. “Upstream” signifies an element that is more 5′ than the reference element. “Downstream” refers to an element that is more 3′ than a reference element.

As used herein, the terms “exon” and “intron” are art-understood terms referring to various portions of genomic gene sequences. “Exons” are those portions of a genomic gene sequence that encode protein. “Introns” are sequences of nucleotides found between exons in genomic gene sequences.

Chromosomal synteny may be used to further distinguish between homologous genes when there is sufficient evolutionary conservation between the genomes that are being compared, e.g. between mammalian species. A “syntenic homolog” has both sequence identity to the reference gene, and has the corresponding chromosomal location in relation to closely linked genes. Syntenic homologs have a high probability of sharing spatial and temporal localization of gene expression, and of encoding proteins that fill equivalent biological roles.

Various methods, well known in the art can also be used to identify genes, variants and variants thereof, for example, sequencing, hybridization assays etc.

As used herein, “variant” of KiflC polypeptides refers to an amino acid sequence that is altered by one or more amino acid residues. The variant may have “conservative” changes, wherein a substituted amino acid has similar structural or chemical properties (e.g., replacement of leucine with isoleucine). More rarely, a variant may have “nonconservative” changes (e.g., replacement of glycine with tryptophan). Analogous minor variations may also include amino acid deletions or insertions, or both. Guidance in determining which amino acid residues may be substituted, inserted, or deleted without abolishing biological activity may be found using computer programs well known in the art, for example, LASERGENE software (DNASTAR).

The term “variant,” when used in the context of a polynucleotide sequence, may encompass a polynucleotide sequence related to KiflC. This definition may also include, for example, “allelic” (as defined above), “splice,” “species,” or “polymorphic” variants. A splice variant may have significant identity to a reference molecule, but will generally have a greater or lesser number of polynucleotides due to alternate splicing of exons during mRNA processing. The corresponding polypeptide may possess additional functional domains or an absence of domains. Species variants are polynucleotide sequences that vary from one species to another.

The resulting polypeptides generally will have significant amino acid identity relative to each other. A polymorphic variant is a variation in the polynucleotide sequence of a particular gene between individuals of a given species. Polymorphic variants also may encompass “single nucleotide polymorphisms” (SNPs,) in which the polynucleotide sequence varies by one base. The presence of SNPs may be indicative of, for example, a certain population with a propensity for a disease state, that is susceptibility versus resistance.

As used herein, “anthrax resistant genes” or “genes” will be used interchangeably unless otherwise specified, and refer are those genes which render susceptible cells resistant to the anthrax lethal toxin as described in the examples and methods. The genes may be wild-type, alleles, variants, mutations, nucleic acid molecules or fragments, thereof.

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogues thereof, such as phosphorothioates and thioesters, in either single stranded form, or a double-stranded helix. Double stranded DNA--DNA, DNA-RNA and RNA--RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA). A “recombinant DNA molecule” is a DNA molecule that has undergone a molecular biological manipulation.

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1967 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

A DNA “coding sequence” is a double-stranded DNA sequence which is transcribed and translated into a polypeptide in a cell in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences and synthetic DNA sequences. If the coding sequence is intended for expression in a eukaryotic cell, a polyadenylation signal and transcription termination sequence will usually be located 3′ to the coding sequence.

Transcriptional and translational control sequences are DNA regulatory sequences, such as promoters, enhancers terminators, and the like, that provide for the expression of a coding sequence in a host cell. In eukaryotic cells, polyadenylation signals are control sequences.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

A coding sequence is “under the control” of transcriptional and translational control sequences in a cell when RNA polymerase transcribes the coding sequence into mRNA, which is then trans-RNA spliced and translated into the protein encoded by the coding sequence.

As used herein, the term “homologous” in all its grammatical forms refers to the relationship between proteins that possess a “common evolutionary origin,” such as for example kinesins, kinesin-like motor proteins. Other examples of common evolutionary origin are proteins from superfamilies (e.g., the immunoglobulin superfamily) and homologous proteins from different species (e.g., myosin light chain, etc.) (Reeck et al., 1987, Cell, 50: 667). Such proteins have sequence homology as reflected by their high degree of sequence similarity.

Accordingly, the term “sequence similarity” in all its grammatical forms refers to the degree of identity or correspondence between nucleic acid or amino acid sequences of proteins that may or may not share a common evolutionary origin (see Reeck et al., supra). However, in common usage and in the instant application, the term “homologous,” when modified with an adverb such as “highly,” may refer to sequence similarity and not a common evolutionary origin.

The term “corresponding to” is used herein to refer to homologous amino acid (or nucleotide) sequences in which the relative positions of the amino acid residues (or nucleotides) is equivalent though the numbering of the amino acid residues or nucleotide bases of the sequences may not be the same.

A “heterologous nucleotide sequence” as used herein is a nucleotide sequence that is added to a nucleotide sequence of the present invention by recombinant methods to form a nucleic acid which is not naturally formed in nature. Such nucleic acids can encode chimeric and/or fusion proteins. Thus the heterologous nucleotide sequence can encode peptides and/or proteins which contain regulatory and/or structural properties. In another such embodiment the heterologous nucleotide can encode a protein or peptide that functions as a means of detecting the protein or peptide encoded by the nucleotide sequence of the present invention after the recombinant nucleic acid is expressed. In still another such embodiment the heterologous nucleotide can function as a means of detecting a nucleotide sequence of the present invention. A heterologous nucleotide, sequence can comprise non-coding sequences including restriction sites, regulatory sites, promoters and the like.

A “vector” is a replicon, such as plasmid, phage or cosmid, to which another DNA segment may be attached so as to bring about the replication of the attached segment. A “replicon” is any genetic element (e.g., plasmid, chromosome, virus) that functions as an autonomous unit of DNA replication in vivo, i.e., capable of replication under its own control.

As used herein the term “stringent conditions” encompasses conditions known in the art under which a nucleotide sequence will hybridize to an isolated and purified nucleic acid molecule. One skilled in the art may vary conditions appropriately. Screening polynucleotides under stringent conditions may be carried out according to the method described in Nature, 313:402-404 (1985). Polynucleotide sequences capable of hybridizing under stringent conditions with the polynucleotides of the present invention may be, for example, allelic variants of the disclosed DNA sequences, or may be derived from other sources. General techniques of nucleic acid hybridization are disclosed by Sambrook et al., “Molecular Cloning: A Laboratory Manual”, 2nd Ed., Cold Spring Harbor Laboratory, Cold Spring Harbor, N.Y. (1984); and by Haymes et al., “Nucleic Acid Hybridization: A Practical Approach”, IRL Press, Washington, D.C. (1985), which references are incorporated herein by reference.

The phrase “high stringency hybridization” refers to conditions that permit hybridization of only those nucleic acid sequences that form stable hybrids in, for example, 0.018 M NaCl at 65 C. (i.e., if a hybrid is not stable in 0.018 M NaCl at 65 C., it will not be stable under high stringency conditions). High stringency conditions can be provided, for example, by hybridization in 50% formamide, 5× Denhart's solution, 5× SSPE, 0.2% SDS at 42 C., followed by washing in 0.1× SSPE, and 0.1% SDS at 65 C.

The phrase “low stringency hybridization” refers to conditions equivalent to hybridization in 10% formamide, 5× Denhart's solution, 6× SSPE, 0.2% SDS at 42 C., followed by washing in 1× SSPE, 0.2% SDS, at 50 C. Denhart's solution and SSPE (see, e.g., Sambrook et al., Molecular Cloning, A Laboratory Manual, Cold Spring Harbor Laboratory Press, 1989) are well known to those of skill in the art as are other suitable hybridization buffers.

High stringency hybridization conditions and low stringency hybridization conditions are also described in Example 3, which follows.

As defined herein, the term “standard viability assay” refers to the following protocols which measure the relative survival rates of test cells, such as between cells of different test subjects exposed to LeTx or as between test cells exposed to a candidate therapeutic agent and control cells no so exposed. Such standard viability assays include, without limitation:

a) Neutral red uptake, wherein the living cells take up the dye, rather than excluding the dye and the percentage of cells containing dye is then measured in each sample and compared with results obtained in other samples, which may include control samples;

b) Dye exclusion, which comprises contacting treated cells with a dye (Trypan Blue) which is excluded by live cells, wherein dead cells which take up the dye are counted and percent viability thereby determined for comparison with values observed in other samples, which may include control samples;

c) Direct visualization (microscope); and

d) Use of a modified MTT (3-[4,5-dimethylthiazol-2-yl]-2,5-diphenyltetrazolium bromide [Thiazolyl Blue]) Cytotoxicity Assay (Sigma). Additional known methods can be employed.

As used herein, “resistance” refers to the viability of cells when incubated with LeTx, results in the death of no more than about 10% of treated cells, after normalization against the death rate observed in untreated controls, as measured by viability assays.

As used herein, the term “fragment or segment”, as applied to a nucleic acid sequence, gene or polypeptide, will ordinarily be at least about 5 contiguous nucleic acid bases (for nucleic acid sequence or gene) or amino acids (for polypeptides), typically at least about 10 contiguous nucleic acid bases or amino acids, more typically at least about 20 contiguous nucleic acid bases or amino acids, usually at least about 30 contiguous nucleic acid bases or amino acids, preferably at least about 40 contiguous nucleic acid bases or amino acids, more preferably at least about 50 contiguous nucleic acid bases or amino acids, and even more preferably at least about 60 to 80 or more contiguous nucleic acid bases or amino acids in length. “Overlapping fragments” as used herein, refer to contiguous nucleic acid or peptide fragments which begin at the amino terminal end of a nucleic acid or protein and end at the carboxy terminal end of the nucleic acid or protein. Each nucleic acid or peptide fragment has at least about one contiguous nucleic acid or amino acid position in common with the next nucleic acid or peptide fragment, more preferably at least about three contiguous nucleic acid bases or amino acid positions in common, most preferably at least about ten contiguous nucleic acid bases amino acid positions in common.

Other aspects of the invention are disclosed infra.

### BRIEF DESCRIPTION OF THE DRAWINGS

FIGS. 1A and 1B show a diagrammatic presentation of the genomic comparison of the mouse Ltxsl interval and syntenic human region.

FIG. 2A is a schematic representation of mouse KiflC. Predicted conserved domains are shown as gray boxes. The black boxes represent additional regions identified in the human homologue of KiflC (Dorner et al., 1999, J. Biol. Chem. 274: 33654-33660). The arrows show the positions of the observed missense mutations. FIG. 2B is a diagram showing the Neighbor-joining distance-based phylogram of KiflC sequence.

FIG. 3 is a graph showing the viability of macrophages incubated with BFA and LeTx. Data represents the average of 4 independent experiments ± Standard Error of the Mean (SEM). Asterisk denotes a significant difference from non-BFA treated C57BL/6J cells, p<0.0001. Plus sign denotes a non-significant difference from non-BFA treated C3H/HeJ cells, p>0.25.

FIGS. 4A and 4B present a a graph showing that expression of a resistance allele of KiflC partially rescues susceptible primary macrophages from LeTx-induced cytolysis. The asterisk denotes a significant difference from control-GFP transduced cells, p<0.01.

FIG. 5 is an immunoblot showing that LF is active in resistant and susceptible macrophages.

FIGS. 6A-6U shows nucleic acid sequences involved in resistance or susceptibility to the adverse effects of LeTx (SEQ ID NOS: 1 through 23).

### DETAILED DESCRIPTION OF THE INVENTION

The invention provides diagnostic and therapeutic methods and agents for treatment against Bacillus anthracis. Methods also are provided to identify agents useful for such methods.

We observed that inbred mouse strains exhibit striking differences in the susceptibility of their cultured macrophages to the effects of LeTx. Genetic differences between a susceptible strain (C3H/HeJ) and a resistant strain (C57BL/6J) have been exploited to map a single gene (named Ltxsl) that controls this phenotype to mouse Chromosome 11 (Roberts et al., 1998, Mol. Microbiol. 29: 581-591; Watters and Dietrich, 2001, Genomics, 73: 223-231).

As described in the Examples below, we now have identified by positional cloning the Ltxsl gene as KiflC. KiflC encodes a kinesin-like motor protein of the UNC104 subfamily. A partial cDNA sequence for mouse KiflC has been reported (Nakagawa et al., 1997, Proc. Natl. Acad. Sci. U.S.A. 94 (18): 9654-9659; Genbank accession number AB001456). The invention is based on our observations that mutations in KiflC are responsible for the differences in susceptibility of inbred mouse macrophages to LeTx, and that proper KiflC function is required for LeTx resistance. The syntenic human chromosomal region has been characterized, and human KiflC sequences have been reported (Dorner et al., 1998, J. Biol. Chem., 273 (32): 20267-20275; Ishikawa et al., 1998, DNA Res. 5 (3): 169-176; Genbank accession numbers NM_{—}006612 and U91329). Since the LeTx-mediated proteolysis of map kinase kinase 3 occurs even in resistant cells, KiflC does not affect cellular entry or processing of LeTx, and likely influences events occurring later in the intoxication pathway.

Kinesins comprise a ubiquitous, conserved family of over 50 proteins that can be classified into at least 8 subfamilies based on primary amino acid sequence, domain structure, velocity of movement, and cellular function (reviewed in Moore and Endow, 1996, Bioassays, 18: 207-219; and Hoyt, 1994, Curr. Opin. Cell Biol. 6: 63-68). The prototypical kinesin molecule is involved in the transport of membrane-bound vesicles and organelles. Kinesin is also important in all cell types for the transport of vesicles from the Golgi complex to the endoplasmic reticulum. This role is critical for maintaining the identity and functionality of these secretory organelles.

The prototypical kinesin molecule is a heterotetramer comprised of two heavy polypeptide chains (KHCs) and two light polypeptide chains (KLCs). The KHC subunits are typically referred to as “kinesin.” KHC is about 1000 amino acids in length, and KLC is about 550 amino acids in length. Two KHCs dimerize to form a rod-shaped molecule with three distinct regions of secondary structure. At one end of the molecule is a globular motor domain that functions in ATP hydrolysis and microtubule binding. Kinesin motor domains are highly conserved and share over 70% identity. Beyond the motor domain is an a-helical coiled-coil region which mediates dimerization. At the other end of the molecule is a fan-shaped tail that associates with molecular cargo. The tail is formed by the interaction of the KHC C-termini with the two KLCs.

The nematode UNC-104 kinesin-like protein defines a distinct kinesin subfamily whose members may function monomerically (Moore and Endow, supra). Members of this subfamily are important for synaptic transport and mitochondrial translocation and are characterized by movement at relatively high velocities of about 40 to 90 microns/minute. Nematodes with mutations in the UNC-104 gene exhibit defects in locomotion and feeding behaviors, and at the molecular level, in synaptic vesicle transport.

The kinesin protein family consists of some 45 members. All kinesins contain certain highly conserved amino acids in their ATP- and microtubule-binding domains (together, these domains are said to comprise the “motor” of the kinesin). The mammalian kinesins have been grouped into several subfamilies based on phylogenetic analyses of their protein sequences (Miki et al., 2001, Proc. Natl. Acad. Sci. U.S.A. 98: 7004-7011). They can also be classified according to the position of the motor domain in the polypeptide (N-terminal or “KinN”, C-terminal or “KinC”, and internal or “KinI”). Interestingly, the KinN, KinC, and KinI proteins can also be distinguished functionally, with the KinN proteins catalyzing motility toward the “plus” end of microtubules, the KinC proteins generating movement toward the “minus” end, and KinI proteins possessing microtubule destabilizing activity. Several of the motile kinesins have been implicated in the movement of organelles, complexes of proteins or RNAs, and chromosomes or spindles (reviewed in Miki et al., 2001, supra).

Each of the kinesins differ greatly from the others in their non-motor portions. In general, these divergent regions are believed to facilitate distinct mechanisms of regulation and binding of molecular “cargo”. However, the mechanisms of motor regulation and cargo attachment and release remain poorly described for the vast majority of kinesin motors. Since it is widely believed that each kinesin possesses some relatively unique role in cellular transport and physiology, a major challenge for the kinesin field lies in the identification of the fundamental cargo binding and regulatory properties of these motors (Verhey and Rapoport, 2001, TIBS 26: 545-550; Bloom, G. S. 2001. “The UNC-104/KIF1 family of kinesins.” Curr. Opin. Cell Biol. 13: 36-40).

Homology-based screens have uncovered several homologs of Unc-104 in many other species, including mammals. Two of the best studied mammalian examples are Kiƒl A and Kiƒl B (Okada et al 1995; Nangaku et al 1994). The proteins encoded by Kiƒl A, Unc-104, and one of the Kiƒl B alternative splice forms (Kiƒl B) share extensive homology for the entire protein, including an ≈600 amino acid N-terminal region that encompasses the motor domain and an approx. 100 amino acid forkhead homology associated (FHA) domain. FHA domains, the distinctive feature of the Unc-104/Kifl kinesin subfamily (Westerholm-Parvinen et al., 2000, FEBS Letters 486: 285-290), were first described in a group of forkhead transcription factors, and are protein modules that have been found in over 100 different proteins (Li et al., 2000, J. Cell Sci. 113: 4143-4149). Many FHA domains have been shown to bind to peptide sequences containing phosphothreonine, phosphoserine, or possibly phosphotyrosine residues. The role of the FHA domain in Unc-104/Kifl kinesins is unknown, but reasonable speculation includes mediating homo- or hetero-interactions with proteins involved with motor regulation or cargo binding.

Human KIFl C was originally identified in a 2-hybrid screen for proteins that interact with the ezrin-like domain of protein tyrosine phosphatase D1 (PTPD1) (Dorner et al., 1998, J. Biol. Chem. 273: 20267-20275). KIFl C encodes a widely expressed 1103 amino acid protein that has extremely high homology to Kifl A, Kifl Bα, and Kifl Bβ for the first half of the protein. This region of high homology encompasses the motor and FHA domains, placing KIFl C firmly in the Unc-104/Kifl family. Interestingly, the C-terminal half of KIFl C is also highly homologous to the C-terminal half of Kifl Bα, but not to Kifl A and Kifl Bβ. As previously stated, Kifl Bα was previously shown to be capable of binding to and transporting mitochondria, although biochemical fractionation experiments rule out the possibility that KiflC transports mitochondria (see below). The cargo of KiflC is currently unknown.

Dorner et al found that the physical interaction between KIFl C and PTPD1 is mediated by a small region (amino acids 714-809) in the C-terminal half of KIFl C. Furthermore, this interaction is specific to PTPD1; other ezrin-domain containing tyrosine phosphatases are unable to bind to KIFl C. Interestingly, KIFl C can itself be phosphorylated, both on as yet unmapped tyrosines, and on serine 1092 (Dorner et al 1998, supra; Dorner et al., 1999, J. Biol. Chem. 274: 33654-33660). While it is unclear what role the tyrosine phosphorylations play in the regulation of KIFl C, it seems that the serine phosphorylations are important regulators of binding 14-3-3 protein adaptors (Dorner et al 1999, supra). The relationship of these 14-3-3 adaptors to cargo binding and motor function is unclear.

The discovery of new allelic variants of a gene that encodes a kinesin-like motor protein satisfies a need in the art by providing new compositions which are useful in the diagnosis, prevention, and treatment of individuals susceptible to, or infected with the causative agent of anthrax.

The present invention has important advantages for use in diagnostic screening, drug therapy, drug design and/or discovery and protection against the adverse (lethal) effects of LeTx as it does not contain any anthrax toxins and may be capable of protecting against airborne spores, used for example in biological warfare.

Two fully functional variants of KiflC are required for macrophage resistance to LeTx, while disruptions of KiflC function can induce LeTx susceptibility. In addition, Lethal Factor (LF) is proteolytically active in the cytosol of resistant macrophages, demonstrating that KiflC is not involved in LeTx uptake or activation. Without wishing to be bound by any theory, we believe that KiflC functions downstream in the intoxication pathway, and serves to protect the macrophage from signaling events induced by LF proteolysis of target proteins.

KiflC is a ubiquitously expressed gene encoding an 1100 amino acid kinesin-like motor protein of the UNC104 subfamily that is likely to be involved in the intracellular transport of molecular cargo. The human homolog of KiflC has been implicated in retrograde vesicular transport from the Golgi apparatus to the endoplasmic reticulum (Dorner et al., 1998, J. Biol. Chem., 273: 20267-20275).

KiflC is the only gene in the Ltxsl critical interval that contains mutations between susceptible and resistant inbred mouse strains. As described in the Examples that follow, methods for the identification of genes that influence anthrax susceptibility and useful allelic variants thereof are shown. Methods also are described for the diagnostic screening of subjects for anthrax sensitivity or resistance, as well as for the identification of novel therapeutic compositions aimed at the prevention or treatment of anthrax infection. In addition, procedures are shown that alter the cellular localization of KiflC induce susceptibility in normally resistant macrophages. Ectopic expression of a resistant allele of KiflC rescues normally susceptible cells also is shown.

In the Examples below, the following materials and methods were employed as indicated:

Macrophage Culture

Primary macrophages were differentiated from bone marrow cells following a published protocol (Celada et al., 1984, J. Exp. Med. 160: 55-74). Femurs were flushed with bone marrow media (BMM: RPMI Medium 1640 containing 20% Heat inactivated fetal bovine serum, 30% L-cell conditioned media as a source of granulocyte/macrophage colony stimulating factor (Roberts et al., 1998, Mol. Microbiol. 29: 581-591), 200 μM L-glutamine, 10 μg/ml Streptomycin, and 10 units/ml Penicillin) to obtain approximately 4×10⁶ cells. These cells were then cultured in BMM for 6-7 days in 100 mm polystyrene petri dishes that allow weak adherence of macrophages and easy harvesting by scraping.

Genomic Sequencing

We have previously reported the generation of 1.1X sequence coverage of the Ltxsl interval (Watters and Dietrich, 2001, Genomics 73: 223-231). In order to generate complete genomic sequence of the Ltxsl interval, we sequenced the following BAC clones, listed by accession number of their available sequence, to a sequence coverage of ≧4.6X, with a cumulative Phred score of ≧20 for each base: AC013775, AC090293, AC091476, AC027185, AC026912, AC079221, and AC036122. The BAC clones AC091476, AC027185, AC026912, and AC079221 were obtained from the CITB BAC library (Research Genetics; derived from strain 129S3), whereas the BAC clones AC013775, AC090293 and AC036122 were obtained from the RPCI-23 BAC library (Research Genetics; derived from strain C57BL/6J). Sequencing of these BAC clones was performed using a shotgun sequencing strategy. BAC DNA isolation, library construction, template preparations, sequencing reactions and sequence assembly was done as described in (Lander et al., 2001, Nature, 409: 860-921). Dot-plots comparing the mouse and human syntenic intervals were generated using PipMaker (Schwartz et al., 2000, Genome Res., 10: 577-586).

Transcript Mapping

In order to identify all candidate genes, our mouse genomic sequence was compared to the near complete genomic sequence of the syntenic human interval. In addition to BLAST searches and comparison to public and private genome annotations, the gene finding program GENSCAN was used to identify genes in the mouse and human regions. PCR assays and oligo probes were developed from the 5′ and 3′ ends of any genes or ESTs which were predicted by GENSCAN, and any transcripts to which our genomic sequence showed ≧90% sequence identity over a length of at least 100 bp. The presence or absence of these transcripts was then confirmed by PCR amplification of BAC DNA or hybridization to BAC Southern blots. All BAC Southern blots were performed as previously described (Watters and Dietrich, 2001, Genomics 73: 223-231).

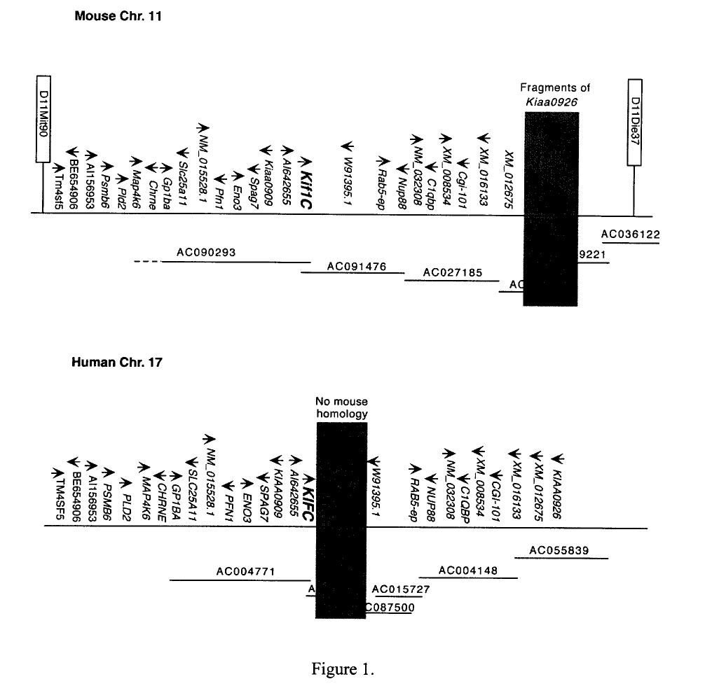

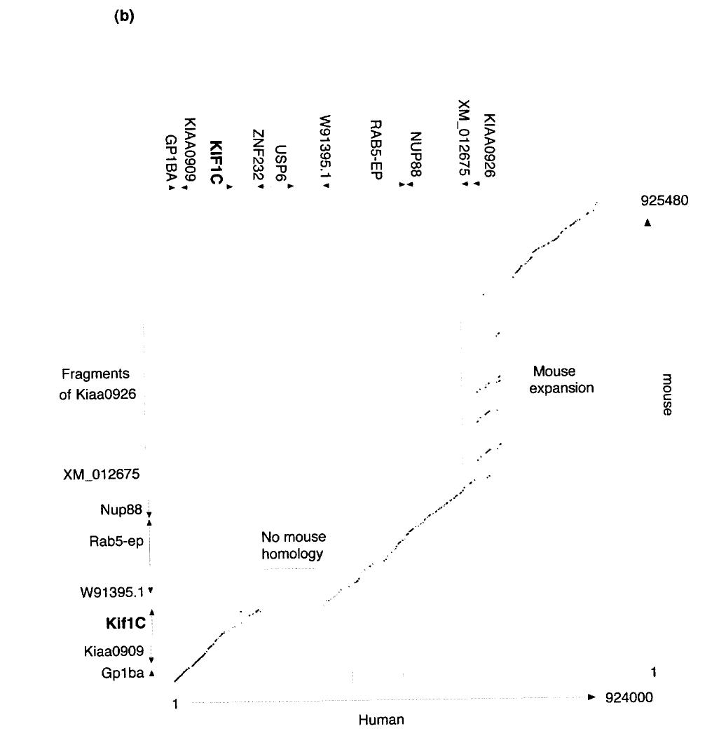

Some gene homologies were detected that are not shown in FIG. 1A. Three genes annotated in the Celera mouse genome assembly of the Ltxsl region have been excluded due to their homology to repetitive elements, or the lack of homology to any sequence in available public databases. The Celera ID of these genes are: mCG66014, mCG57736 and mCG21191. One gene annotated in the Celera human genome assembly was excluded because it is not annotated in the publicly available human genome assembly, and it is not present in the mouse Ltxsl interval. The Celera ID of this gene is hCG1643917. In addition, an intronless pseudogene of the mouse ribosomal protein L37A was detected proximal of Rab5-ep, but is not included due to its presumed lack of function. Finally, numerous ESTs were not included on the map due to their homology to interspersed repetitive elements, or because they encoded ORFs of less than 140 base pairs. Nevertheless, these ESTs were analyzed for expression or sequence differences between resistant and susceptible mouse strains, and no differences were found. The accession numbers of these mouse ESTs are: AI415455, AK018106, AA797627, NM_{—}014922, NM_{—}021730, AA756428, and AA050425.

Analysis of Candidate Genes

Expression of all candidate genes was analyzed by RT-PCR and Northern blot analysis between the resistant strain C57BL/6J and the susceptible strain C3H/HeJ. Total macrophage RNA was extracted from primary macrophages using the Rneasy Midi kit (Qiagen). RT-PCR was performed using the SuperScript One-Step RT-PCR system (Gibco BRL) using 100 ng of total RNA as the amount of template, and the following cycling parameters: An initial 30 minute incubation at 50° C. followed by 40 cycles of 94° C. for 30 seconds, 55° C. for 30 seconds, and 72° C. for 1 minute. Northern blots were performed exactly as described in (Sambrook et al., 1989, Molecular cloning: A laboratory manual, Cold Spring Harbor Laboratory Press, Cold Spring Harbor, N.Y.).

The entire coding sequence of all candidate genes was amplified from the resistant strain C57BL/6J and the susceptible strain C3H/HeJ by RT-PCR as described above, and sequenced. 500 ng of template DNA was sequenced using ABI Big Dye terminator chemistry (Perkin Elmer) according to the manufacturer's specifications. The reactions were performed in an MJ Research thermal cycler (PTC-225). An ABI 3700 was used for detection. DNA sequences were extracted using ABI Data Collection software (Perkin Elmer).

Phenotype Analysis of the CAST/Ei×C57BL/6J F2 Intercross

A 44 animal CAST/Ei×C57BL/6J F2 intercross was analyzed in order to map the LeTx susceptibility phenotype in these strains. Macrophages were harvested as stated above, and phenotyped as described in (Roberts et al., 1998, Mol. Microbiol. 29: 581-591). Using the program MAPMAKER (Lander et al., 1987, Genomics 1: 174-181), LeTx susceptibility maps to D11Mit90, D11Mit320, and D11Die37 at a LOD score of 7.86 in a 44 animal CAST/Ei×C57BL/6J F2 intercross. LeTx susceptibility in this intercross is linked to no other marker at a LOD score higher than 1.7.

Phylogenetic Analysis of KiflC Sequence

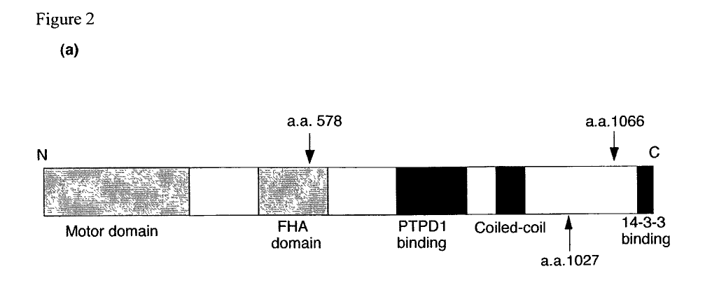

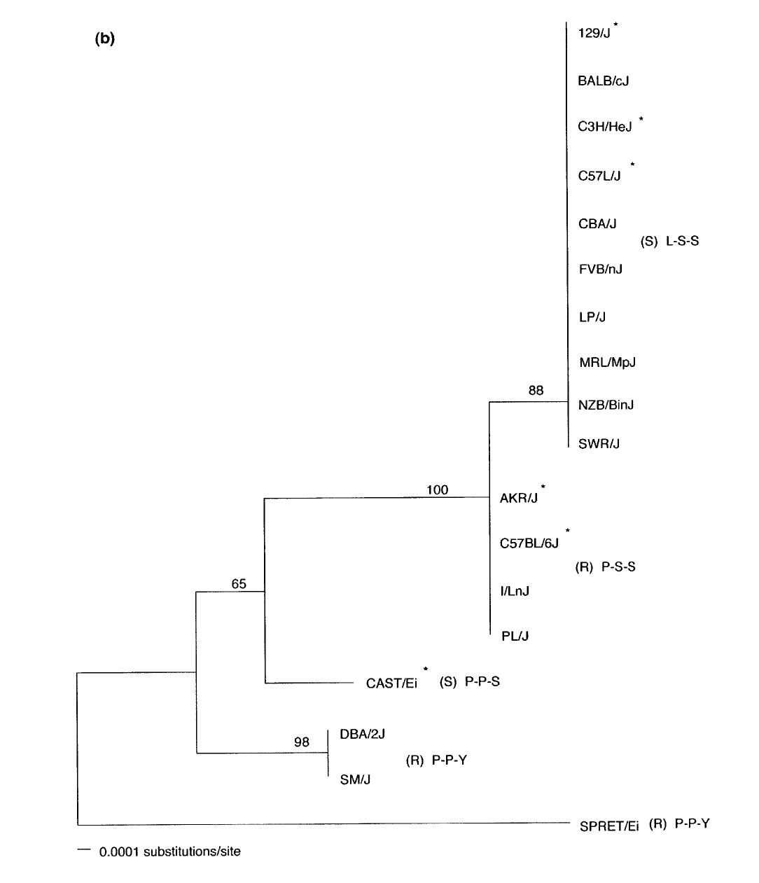

The neighbor-joining, distance-based phylogram shown in FIG. 2B was constructed using the PAUP 4.0β8 (Swofford, 1998, Phylogenetic Analysis Using Parsimony, 4th ed., Sinauer Associates, Sunderland, Mass.) software package, with SPRET/Ei defined as the outgroup. The Kimura 2-paramater calculation was used to generate the distance matrix.

Brefeldin-A Treatment

Primary macrophages were harvested as stated above, and approximately 4×10⁵ cells were plated in 96 well dishes in BMM. 5 μg/ml BFA was added to the indicated samples, and cells were incubated for 20 minutes. 1 μg/ml PA and 100 ng/ml LF were then added, and the cells were incubated for 4 hours at 37° C. Macrophages were then assayed for viability as described by Roberts et al., supra.

Production of Retrovirus

To produce the KiflC^{B6} vector, the C57BL/6J allele of the KiflC cDNA was cloned into the pBABE-MN-IRES-E-GFP vector (gift of Dr. Gary Nolan, Stanford University) derived from the Moloney Murine Leukemia Virus (MMLV), where it is linked to an internal ribosome entry site (IRES) to drive the translation of GFP. Retrovirus was produced by transiently transfecting constructs, using lipofectamine (Gibco BRL), into a stable human-derived packaging cell line that allows for the replacement of the retroviral envelope glycoprotein by the vesicular somatitis virus G protein (VSV-G) (Ory et al., 1996, Proc Natl Acad Sci U.S.A. 93: 11400-11406). This packaging cell line was maintained in DMEM media (Gibco BRL) containing 25 mM HEPES buffer, 10% fetal calf serum, 200 μM L-glutamine, 10 μg/ml Streptomycin 10 units/ml Penicillin, 1 μg/ml Tetracycline, 2 μg/ml Puromycin, and 0.3 mg/ml G418. After transfection, virus was obtained by incubating the cells in the following media, and collecting the media after 168 hours: DMEM containing 25 mM HEPES buffer, 10% fetal calf serum, 200 μM L-glutamine, 10 μg/ml Streptomycin and 10 units/ml Penicillin.

Analysis of Transduced Mouse Macrophages

Primary macrophages were harvested as stated above. Mice were pretreated with 5-fluororuacil (200 mg/kg intravenously) 4 days prior to bone marrow harvest. Two days after bone marrow harvest, the BMM was aspirated, and 10 mls of the previously collected, virus-containing media was added to the cells. Polybrene (Sigma) was also added at a final concentration of 8 μg/ml to increase transduction efficiency. Cells were incubated for 4 hours at 37° C. Macrophages were transduced with the control-GFP construct (C3H/HeJ-GFP) or the KiflC^{B6} construct (C3H/HeJ-Resistant). The virus-containing media was then aspirated, and the cells were grown for 10 days in BMM. Transduced macrophages were harvested by scraping, and sorted on the basis of fluorescence. Cells expressing GFP were assayed for viability in response to challenge with LeTx. Approximately 1×10⁴ cells were plated in 96 well dishes in BMM with 1 μg/ml PA and 100 ng/ml LF for 4 hours at 37° C. Macrophages were then assayed for viability as described in (Roberts et al., 1998, supra).

Analysis of Map Kinase Kinase 3 Cleavage

A polyclonal antibody generated against a C-terminal epitope of map kinase kinase 3 (MKK3) was purchased from Santa Cruz Biotechnology (catalog number SC-961). Primary macrophages from C57BL/6J and C3H/HeJ mice, differentiated from bone marrow cells according to published protocols (Roberts et al., 1998, supra; Celada et al., 1984, J. Exp. Med. 160: 55-74), were grown to confluence in 6 well dishes (Costar). Cells were either processed without toxin treatment, or after a one hour incubation in 10 μg/ml PA and 1 μg/ml LF. Protein extracts were prepared from the cells by resuspending in 200 μl SDS sample buffer (50 mM DTT, 62.5 mM Tris pH=7.0, 2% weight/volume SDS, 10% glycerol, and 0.1% Bromophenol Blue). Proteins were separated on a 10% SDS-PAGE gel, and transferred to PDVF membranes using a Bio-Rad wet transfer apparatus. Membranes were probed with a 1:3000 dilution of the MKK3 antibody, washed and processed with the enhanced chemiluminescence detection kit (Amersham Pharmacia) according to manufacturer's suggested protocol.

Human Test Subjects for Macrophage Screening

Approximately 20 ml to 50 ml of venous peripheral blood is drawn from each human test subject, which subject is to be a healthy individual, based on one or more of criteria that include apparent physical health, answers to questions posed by the administrator of the diagnostic screening procedure and the results of physical examination or other medical testing procedures. The risks of venous blood collection in the amount specified are minimal.

The blood so drawn then is used as a source of cells to determine macrophage susceptibility to anthrax LeTx, and also to create lymphoblastoid cell lines as a source of genetic material for use in the correlation of the observed susceptibility phenotype with particular allelic variants. Identifying information ideally is kept confidential, consistent with accepted biomedical ethics, and is used only to guard against duplicate blood donations.

Analysis of Human Macrophage

To isolate macrophages, approximately 20 ml of blood from each healthy human subject is collected in ACD tubes. The blood is kept on ice until it is layered onto a Ficoll gradient. The mononuclear layer then is isolated and half of it is plated onto tissue culture plastic in RPMI+10% FCS+recombinant human M-CSF. The remainder of the mononuclear cell fraction is frozen for later transformation with EBV for the production of lymphoblastoid cell lines to be used in genetic studies. After a week's differentiation, the cells are harvested and replated for intoxication assays, done with LeTx. Mouse cells are intoxicated contemporaneously as controls. The viability of the cells is measured using standard methods, such as neutral red staining and microscopy, as described above. This method is used to determine with reasonable accuracy the macrophage susceptibility of humans. In control studies, we have found that mouse macrophages obtained by treatment with recombinant mouse M-CSF have LeTx susceptibility phenotypes consistent with the behavior of macrophages isolated using different methods.

Additional intoxications may be performed to further characterize any resistance phenotype that is observed, such as to titrate levels of resistance. Human macrophage cells also may be tested with LFn-DTA, which is a derivative of LF that uses PA to gain entry to cells, but kills them with the catalytic subunit of diphtheria toxin. This toxin can give us some information about whether any given individual's macrophages are resistant because their mechanisms for PA-mediated toxin uptake are defective. Since genetically resistant mouse macrophages can be made susceptible with brefeldin A treatment, human cells also may be intoxicated according to the invention in the presence of brefeldin A for further phenotypic analysis.

Macrophages from a number of different human individuals from different racial and ethnic backgrounds, and of different ages, are advantageously screened according to the invention for susceptibility or resistance to LeTx, thereby enabling classification as “resistant” or “susceptible” the allelic variants that exist among humans, even such variants as may be found at very low frequencies or in specific sub-populations.

In addition to screening methods described herein that entail the use of Ficoll gradients, magnetic bead purification of cells via commercially-available methods (for example Dynal Beads) to select for those containing known mononuclear phagocyte specific marker proteins may be employed to enable rapid screening of large numbers of test subjects.

Human Test Subjects for Genetic Screening

In addition to the methods described above, it may be desirable to screen for allelic variants of genes that mediate anthrax susceptibility in individuals already known to be susceptible to anthrax infection; namely, individuals who have suffered anthrax infection. Once their genotypes are determined, variants of anthrax-susceptibility genes identified according to the invention may be compared with those of the general population or with those of individuals in whom macrophage screening has revealed a LeTx-resistant phenotype to determine which variants correlate with anthrax susceptibility.

Only a small number of individuals have been diagnosed as having pulmonary anthrax, of whom an even smaller number survive. Living individuals, where identified, may provide blood samples for testing. To the extent that frozen or otherwise preserved biological samples derived from deceased anthrax patients, they may serve as a source of nucleic acids for screening. In addition to those individuals who have suffered pulmonary anthrax, a relatively large number of people, most particularly farmers, hunters and others who live or work in rural areas or in proximity to wool-processing operations, suffer cutaneous anthrax infection, a form of the disease that only rarely is fatal. Such individuals may be recruited and genetically screened.

The following non-limiting examples are illustrative of the invention. All documents cited in these Examples and in the above description of methods used therein are incorporated herein by reference.

### Example 1

Identification and characterization of a gene that mediates anthrax susceptibility:

An illustrative example of the methods to identify and characterize the KiflC gene and its product is provided here. This example is not meant to limit or construe the invention in any way but provides a general method of identifying and isolating genes and allelic variants thereof useful in the invention.

Identification of Candidate Genes

In order to identify all positional candidate genes, a virtually complete genomic sequence was generated for the mouse Ltxsl interval using BAC clones derived from strains C57BL/6J and 129S3 (129S3 is a susceptible strain in which the LeTx susceptibility phenotype maps to the Ltxsl interval; Watters and Dietrich, 2001, Genomics 73: 223-231). To aid in the identification of candidate genes, this mouse sequence was compared to the publicly available genomic sequence data of the syntenic human interval. As shown in FIG. 1A, the structure of the mouse and human regions are very similar, with a few notable exceptions. First, there is a break in synteny homology in the middle of the interval, in which three human genes are present, but no homologous mouse genes can be detected. Additionally, the two most distal genes, XM_{—}012675 and KIAA0926, appear to be non-functional in the mouse. Comparative sequence analysis revealed that the first three exons of KIAA0926, as well as the coding region of XM_{—}012675, are not present in the genome of strain 129S3, while RT-PCR and Northern blot analysis failed to detect expression of XM_{—}012675 or KIAA0926 in susceptible or resistant mouse macrophages. Furthermore, BLAST searches revealed that there are no mouse ESTs homologous to either XM_{—}012675 or KIAA0926, indicating that these two genes are likely not expressed in the mouse.

XM_{—}012675 and KIAA0926 are present in an area of the mouse genome that appears to have undergone a repetitive expansion, with at least 3 partial copies of KIAA0926 being present in strain 129S3 (see FIG. 1B). The possibility that this genomic expansion represents the Ltxsl mutation can be ruled out for two reasons: 1) The presence or absence of this expansion does not perfectly correlate with LeTx susceptibility (Watters and Dietrich, 2001, supra), and 2) LeTx susceptibility maps to the Ltxsl interval in a recombinant inbred strain panel derived from strains C57L/J and AKR/J, two strains which have opposite phenotypes, but an identical expansion (Watters and Dietrich, 2001, supra).

The results are shown in FIG. 1, which is a genomic comparison of the mouse Ltxsl interval and syntenic human region. FIG. 1A presents a gene map of the mouse chromosomal region that contains Ltxsl and the syntenic human chromosomal region. The mouse Ltxsl interval is covered by draft quality sequence (≧4.6×sequence coverage, with a cumulative Phred score of ≧20 for each base). Genes are represented by their accepted abbreviations. In cases where no accepted abbreviation exists, GenBank accession numbers of transcripts homologous to the identified gene are listed. The centromere is oriented toward the left of the figure, and an arrow represents the direction of gene transcription. KiflC is indicated in bold. The genetic markers that flank the Ltxsl region are shown inside vertical rectangles (Watters and Dietrich, 2001, supra). The horizontal bars beneath the genes represent BAC clones (listed by the accession number of their available sequences) that were used to construct the ordered and oriented sequence constructs depicted in FIG. 1B. The dashed portion of mouse BAC AC090293 was not included in the ordered and oriented mouse sequence construct. FIG. 1B presents a dot plot, generated using PipMaker (Schwartz et al., 2000, Genome Res. 10: 577-586), that shows the relative homology between the human and mouse sequence constructs. The human interval is displayed left to right, and the mouse interval is displayed bottom to top; the lengths of the intervals are indicated in base pairs. An area of homology is indicated by a dot at the corresponding coordinate. Hash marks along the bottom of the plot represent sequence gaps in the human assembly. Landmark genes are indicated according to their position. The region of no mouse homology and the mouse expansion are indicated within rectangles at their respective positions. The mouse expansion appears to be repetitive, as multiple contiguous regions of the strain 129S3 genome are homologous to one stretch of the human genomic assembly.

Identification of Mutations in KiflC

Analysis of the expression patterns and coding sequence of all candidate genes in the Ltxsl interval revealed that only one gene, KiflC, exhibited any polymorphisms between resistant and susceptible strains.

KiflC contains a number of domains that are likely to be important for its function (see FIG. 2A). The data show a C→T transition in KiflC that results in a proline to leucine substitution at position 578 in the susceptible strain C3H/HeJ relative to the resistant strain C57BL/6J. This substitution alters an evolutionarily conserved proline in the Forkhead Homology Association (FHA) domain, which is thought to be involved in protein-protein interactions regulated by phosphorylation (Dorner et al., 1998, J. Biol. Chem., 273: 20267-20275; Westerholm-Parvinen et al., 2000, FEBS Letters 486: 285-290). This evolutionary conservation suggests that the proline at position 578 is functionally important in KiflC, and substitution of this amino acid is likely to have a deleterious effect on the function of KiflC.

To correlate KiflC genotype with the LeTx susceptibility phenotype, the coding region of KiflC in 16 other inbred strains, was sequenced. The P⁵⁷⁸→L⁵⁷⁸ substitution observed between C57BL/6J and C3H/HeJ was by far the most common polymorphism encountered, with 12 other strains exhibiting either the C57BL/6J or C3H/HeJ allele of KiflC. Susceptibility and resistance correlates with L⁵⁷⁸ and P⁵⁷⁸, respectively, in all 12 strains (see FIG. 2B).

Other variants of KiflC were discovered. These additional variants all contained the P⁵⁷⁸ observed in the resistant strain C57BL/6J, with various C-terminal amino acid substitutions. Surprisingly, one of these variants was discovered in strain CAST/Ei, which has the susceptible phenotype. In addition to P⁵⁷⁸, the CAST/Ei allele of KiflC contains a T→C transition that causes the substitution of P¹⁰²⁷ for S¹⁰²⁷. This mutation confers susceptibility, as confirmed by the fact that the LeTx susceptibility phenotype of CAST/Ei indeed maps to the Ltxsl interval (see materials and methods, above).

In addition, three other resistant strains were identified (DBA/2J, SM/J and SPRET/Ei) that have the P⁵⁷⁸ and P¹⁰²⁷ polymorphisms seen in the CAST/Ei allele of KiflC, with an additional AG→TA nucleotide change introducing a S¹⁰⁶⁶→Y¹⁰⁶⁶ substitution. The S¹⁰⁶⁶→Y¹⁰⁶⁶ substitution observed in KiflC, possibly has a functional consequence, as this substitution was only seen in resistant strains (see FIG. 2B).

The neighbor-joining distance-based phylogram shown in FIG. 2B demonstrates the molecular relatedness of the various KiflC variants. The basic architecture of this tree, which separates the commonly used inbred strains from the haplotypes seen in CAST/Ei, DBA/2J, SM/J, and SPRET/Ei, is maintained using sequences throughout the entire Ltxsl interval. The phylogram shows that susceptible and resistant strains always cluster in the same group according to KiflC sequence, and that susceptibility and resistance can be perfectly correlated with the particular combination of amino acids at positions 578, 1027, and 1066. This result is highly suggestive, that the observed KiflC mutations are causative, and not simply in linkage disequilibrium with the true mutation(s), as this would require that the observed KiflC mutations happened to arise on distinct resistance and susceptibility haplotypes in a manner that perfectly correlates with the phenotype. Although positions 1027 and 1066 are not in evolutionarily conserved domains, they are in the C-terminal tail region of KiflC that is thought to participate in cargo binding.

The results are summarized in FIG. 2 which shows the mutations encountered in KiflC. FIG. 2A presents a schematic representation of mouse KiflC. Predicted conserved domains are shown as gray boxes. The black boxes represent additional regions identified in the human homologue of KiflC (Dorner et al., 1999, J. Biol. Chem. 274: 33654-33660). The arrows show the positions of the observed missense mutations. FIG. 2B shows neighbor-joining distance-based phylogram of the KiflC sequence. Strain names are represented at their position on the phylogram, with the corresponding LeTx phenotype shown in parenthesis. S=susceptible, R=resistant. Indicated bootstrap values were obtained with 1000 pseudoreplicates. The combination of amino acids at positions 578, 1027, and 1066 of KiflC is shown to the right of the phenotype for each group. Asterisk denotes a strain for which the LeTx susceptibility phenotype has been mapped to the Ltxsl interval. A parsimony-based method of tree building resulted in an identical tree.

Disruption of KiflC Localization Induces Susceptibility

KiflC localizes to the Golgi apparatus in NIH3T3 cells, and that treatment with the fungal metabolite brefeldin A (BFA) results in a dramatic relocalization of KiflC to the Endoplasmic Reticulum. Reasoning that perturbing the cellular localization of KiflC would alter its ability to perform its molecular function, we challenged C57BL/6J and C3H/HeJ primary bone marrow-derived macrophages with LeTx in the presence of BFA.

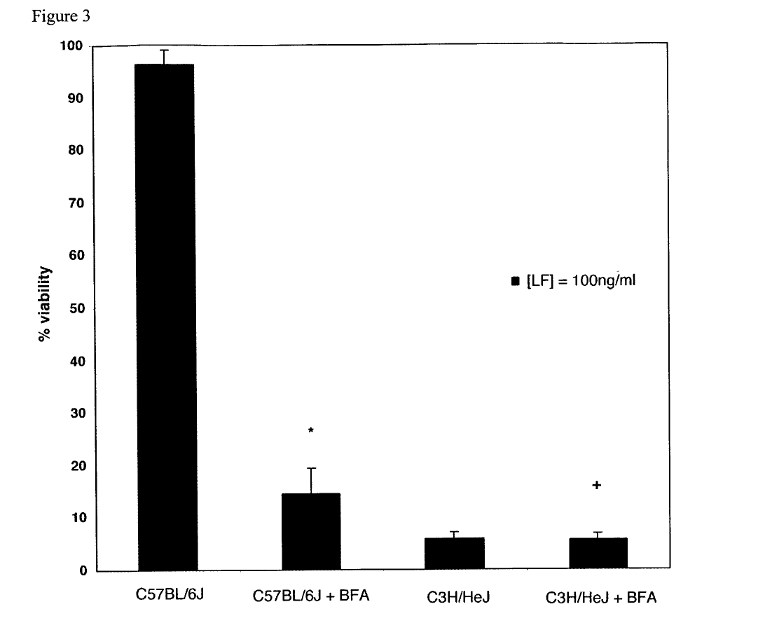

As shown in FIG. 3, intoxication of the cells in the presence of BFA caused the resistant C57BL/6J macrophages to become completely susceptible to intoxication with LF, whereas the susceptibility of C3H/HeJ macrophages was unaltered. Incubation with BFA causes resistant macrophages to become susceptible to the effects of LeTx. BFA alone had no effect on the viability of either resistant or susceptible cells (data not shown). Data represents the average of 4 independent experiments ± Standard Error of the Mean (SEM). Asterisk denotes a significant difference from non-BFA treated C57BL/6J cells, p<0.0001. Plus sign denotes a non-significant difference from non-BFA treated C3H/HeJ cells, p>0.25.

The same experiment was performed using a double-mutant PA (K397D, D425K) that has been shown to prevent the translocation of LF into the cytosol of target cells (Sellman et al., 2001, Science, 292: 695-697). The double mutant PA completely blocked the susceptibility-inducing effect of BFA, demonstrating that this effect of BFA is dependent upon proper internalization and targeting of LF to the cytosol.

KiflC is Not Involved in LeTx Cellular Uptake

It is possible that KiflC is involved in the process of cellular uptake or activation of LF, and that differences in these processes caused by mutations in KiflC could be the mechanism by which resistant macrophages survive LeTx intoxication. To address this, the ability of LF to cleave map kinase kinase 3 (MKK3), a known target of LF proteolysis, in resistant and susceptible macrophages, was assayed.

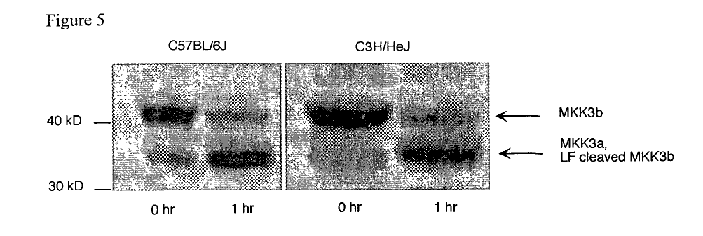

As shown in FIG. 5, the anti-MKK3 antibody detected two distinct bands: a higher molecular weight band corresponding to isoform MKK3b, and a lower molecular weight band corresponding to isoform MKK3a (Moriguchi et al., 1996, J. Biol. Chem. 271: 26981-26988). After a one-hour incubation in LeTx, the lower molecular weight band becomes much more prominent in both resistant and susceptible macrophages. This banding pattern is consistent with that seen in previous demonstrations of MKK3 cleavage by LF, in which MKK3b was cleaved, but MKK3a was not (Vitale et al., 2000, Biochem. J., 352 pt. 3: 739-745). The data suggested that upon cleavage, isoform MKK3b migrates near the same apparent molecular weight as MKK3a. Therefore, the observed LeTx-induced increase in the relative intensity of the lower molecular weight band demonstrates LF cleavage of MKK3b. Since LF is able to enter resistant cells and cleave a known target of proteolysis, resistant variants of KiflC cannot be responsible for a loss of the ability to internalize or activate LF.

The results are summarized in FIG. 5 whereby, LF is active in resistant and susceptible macrophages. C57BL6/J (resistant) and C3H/HeJ (susceptible) macrophages were either untreated (0 hr), or incubated for one hour in the presence of anthrax LeTx (1 hr). Protein extracts were separated by SDS-PAGE, blotted and probed with a polyclonal antibody specific for the C-terminus of MKK3 (see materials and methods). A downward shift of the MKK3b isoform reveals proteolysis by LF.

Particularly important to understanding the mechanisms of lethal toxin activity, the invention provides for the enzyme cleavage activity of the lethal factor. We have confirmed that LF exhibits a map kinase kinase cleavage activity (MKK3) in resistant macrophages (see also Pellizzari et al., 1999, FEBS Letters, 462: 199-204). LF cleaved MKK3 in both resistant and susceptible cells, demonstrating that LF is able to enter the cytosol and function in resistant macrophages. We consider it very unlikely that KiflC participates in the cellular uptake or activation of LF. This is supported by several independent observations that the macrophage resistance of certain mouse strains to LeTx is not due to defects in toxin uptake (Roberts et al., 1998, Mol. Microbiol., 29: 581-591; Swain et al., 1997, Indian J. Biochem. Biophys., 34: 186-91; Friedlander et al., 1993, Infect. Immun. 61: 245-252). Based on this reasoning, we believe that KiflC impacts important physiologic events that are downstream from the initiating LF proteolytic event(s).

The specificity of LeTx for killing macrophages cannot be currently explained by macrophage specificity of any of its known proteolytic targets or downstream effectors. This suggests that LeTx treatment induces a response unique to macrophages, such as a massive inflammatory burst (Hanna et al., 1993, Proc. Natl. Acad. Sci. U.S.A. 90: 10198-10201) (perhaps influenced by the MAPKK cleavage activity attributed to LF), and that KiflC is required to function at very high levels in order to accommodate this stress. By this logic, the observed susceptibility variants of KiflC represent hypomorphs, and the reduced KiflC function in susceptible and heterozygous macrophages renders them unable to protect themselves from the LF-induced hyper-inflammatory response. Resistant macrophages, which harbor two fully functional KiflC alleles, are able to survive this response. It is therefore likely that small kinetic defects in the ability of KiflC to perform its normal function become deadly upon LeTx treatment. Interestingly, haploinsufficiency of Kifl Bβ, another UNC104 kinesin family member, has recently been shown to cause Charcot-Marie-tooth disease type 2A in humans (Zhao et al., 2001, Cell, 105: 587-597), supporting the notion that modest reduction of kinesin function can produce deleterious effects in situations in which the cellular transport machinery is stretched to its limits.

Animal species differ in their susceptibility both to infection by Bacillus anthracis, and to anthrax toxin (Welkos et al., 1986, Infect. Immun. 51: 795-800), and it is possible that differences in homologous KiflC variants are responsible for these variations in susceptibility. All publicly available rat and human KIFl C sequences contain amino acids P-P-Y, respectively, at positions homologous to 578, 1027 and 1066 in mouse KiflC. While this suggests that rat and human macrophages should be resistant to the effects of LeTx, this contradicts previous data demonstrating the susceptibility of primary human and rat macrophages (Beall et al., 1962, J. Bacteriol. 83: 1274-1280; Hanna et al., 1994, Mol. Med. 1: 7-18). However, there are many other sites of missense polymorphism between mouse, rat and human KiflC. Since even subtle disruption of KiflC function can have profound effects on macrophage susceptibility to LeTx, these polymorphisms must be considered as possible sources of rat and human susceptibility.

Identification of additional genes that mediate anthrax susceptibility or resistance

In another embodiment of the invention, nucleic acid sequences encoding KiflC may be used to generate hybridization probes useful in mapping the naturally-occurring genomic sequence, as well as to detect in an individual, or group of individuals, allelic variants of genes that mediate anthrax susceptibility. The sequences may be mapped to a particular chromosome, to a specific region of a chromosome, or to artificial chromosome constructions, e.g., human artificial chromosomes (HACs), yeast artificial chromosomes (YACs), bacterial artificial chromosomes (BACs), bacterial P1 constructions, or single chromosome cDNA libraries (see, e.g., Harrington et al., 1997, Nat Genet. 15: 345-355; Price, 1993, Blood Rev. 7: 127-134; and Trask, 1991, Trends Genet. 7: 149-154).

Fluorescent in situ hybridization (FISH) may be correlated with other physical chromosome mapping techniques and genetic map data (see, e.g., Heinz-Ulrich et al., 1995, in Meyers, supra, pp. 965-968). Examples of genetic map data can be found in various scientific journals or at the Online Mendelian Inheritance in Man (OMIM) site. Correlation between the location of the gene encoding KifC1 on a physical chromosomal map and susceptibility, may help define the region of DNA associated with susceptibility. The nucleotide sequences of the invention may be used to detect differences in gene sequences among resistant, susceptible, or allelic variants in individuals.

In situ hybridization of chromosomal preparations and physical mapping techniques, such as linkage analysis using established chromosomal markers, may be used for extending genetic maps. Often the placement of a gene on the chromosome of another mammalian species, such as mouse, may reveal associated markers even if the number or arm of a particular human chromosome is not known. New sequences can be assigned to chromosomal arms by physical mapping. This provides valuable information to investigators searching for genes of the invention using positional cloning or other gene discovery techniques. Once the genes have been crudely localized by genetic linkage to a particular genomic region, e.g., ataxia-telangiectasia to 11 q22-23, any sequences mapping to that area may represent associated or regulatory genes for further investigation (see, e.g., Gatti et al., 1988, Nature 336: 577-580). The nucleotide sequence of the subject invention may also be used to detect differences in the chromosomal location due to translocation, inversion, etc., among normal, or affected individuals.

The genes identified from individuals are amplified by PCR and sequenced by methods well known in the art. These nucleic acid sequences are then used in the assays described in the examples and materials and methods to correlate the sequence of the genes identified, with the in vitro LeTx response of macrophages isolated from individuals. As more gene sequences and their amino acid sequences are identified, allows for a correlation between the effects of LeTx on macrophages (resistant, intermediate or susceptible) and different gene sequences.

In a preferred embodiment, once genes have been identified, these can be cloned, introduced into cells, by any of the methods described above, allow the expression of the encoded product and then test the cells against LeTx. In this way, if these genes confer protection or do not protect, they can be identified and correlations can be drawn between the genetic information and LeTx effects on cells.

### Example 2

Having identified genes that influence the extent to which an individual is resistant or sensitive to anthrax infection, one can use the invention to correlate allelic variants of such genes with the resistant or susceptible phenotype when macrophages are subjected to the LeTx. The identification of genes which can influence susceptible and resistant individuals, an important first step in determining which nucleic acid sequences render individuals susceptible to the lethal effects of LeTx, is performed according to the methods described above and in Example 1. Detection of KiflC variants that correlate with resistance or susceptibility is accomplished as described hereinbelow.

A preferred embodiment of the invention is to identify genes and/or variants and correlate the effects of the protein encoded by these genes, when macrophages are subjected to the LeTx. The identification of genes which can distinguish between susceptible and resistant individuals is important for distinguishing which nucleic acid sequences render individuals susceptible to the lethal effects of LeTx. Detection of KiflC correlated with resistance or susceptibility is accomplished using methods well known in the art.

The genes identified from individuals are amplified by PCR and sequenced by methods well known in the art. These nucleic acid sequences are then used in the assays described in the examples and materials and methods to correlate the sequence of the genes identified, with the in vitro LeTx response of macrophages isolated from individuals. As more gene sequences and their amino acid sequences are identified, allows for a correlation between the effects of LeTx on macrophages (resistant, intermediate or susceptible) and different gene sequences.

As more genes or variants thereof, are identified, oligonucleotide sequences are generated such as for example SEQ ID NOs 1-23, or fragments thereof, may be employed as probes in the purification, isolation and detection of genes with similar sequences. Identification of a nucleic acid sequence capable of binding to a biomolecule of interest can be achieved by immobilizing a library of nucleic acids onto the substrate surface so that each unique nucleic acid was located at a defined position to form an array. The array would then be exposed to the biomolecule under conditions which favored binding of the biomolecule to the nucleic acids. Non-specifically binding biomolecules could be washed away using mild to stringent buffer conditions depending on the level of specificity of binding desired. The nucleic acid array would then be analyzed to determine which nucleic acid sequences bound to the biomolecule. Preferably the biomolecules would carry a fluorescent tag for use in detection of the location of the bound nucleic acids. Assays using an immobilized array of nucleic acid sequences may be used for determining the sequence of an unknown nucleic acid; single nucleotide polymorphism (SNP) analysis; analysis of gene expression patterns from a particular species, tissue, cell type, etc.; gene identification; etc. Any sequence can then be tested in macrophage viability assays described infra, or any other physical phenotypic criteria such as localization, MAP kinase 3 cleavage patterns and the like.

Other methods to determine the contributions of individual genes and or variants thereof, and their expression products. Genes or variants, thereof, can be isolated to selectively inactivate the native wild-type gene, for example the resistant or susceptibility gene. Techniques are available to inactivate or alter any genetic region to any mutation desired by using targeted homologous recombination to insert specific changes into chromosomal variants. One approach for detecting homologous alteration events uses the polymerase chain reaction (PCR) to screen pools of transformant cells for homologous insertion, followed by screening individual clones (Kim et al., Nucleic Acids Res. 16:8887-8903 (1988); Kim et al, Gene 103:227-233 (1991)). Alternatively, a positive genetic selection approach has been developed in which a marker gene is constructed which will only be active if homologous insertion occurs, allowing these recombinants to be selected directly (Sedivy et al., Proc. Natl. Acad Sci. USA 86:227-231 (1989)). One of the most general approaches developed for selecting homologous recombinants is the positive-negative selection (PNS) method developed for genes for which no direct selection of the alteration exists (Mansour et al., Nature 336:348-352: (1988); Capecchi, Science 244:1288-1292, (1989); Capecchi, Trends in Genet. 5:70-76 (1989)). The PNS method is more efficient for targeting genes that are not expressed at high levels because the marker gene has its own promoter. Nonhomologous recombinants are selected against by using the Herpes Simplex virus thymidine kinase (HSV-TK) gene and selecting against its nonhomologous insertion with the herpes drugs such as gancyclovir (GANC) or FIAU (1-(2-deoxy 2-fluoro-B-D-arabinofluranosyl)-5-iodouracil). By this counter-selection, the number of homologous recombinants in the surviving transformants can be enriched. Such transformants can be correlated with phenotypes as described infra.

### Example 3

Methods for Screening an Individual for Anthrax Susceptibility or Resistance

a. Macrophage Screening of Humans

According to the invention, human subjects are selected and their macrophages assayed for resistance or susceptibility to the effects of LeTx as described above and also as shown below.

Analysis of Human Monocyte-Derived Macrophages for LeTx Susceptibility

The LeTx susceptibility phenotype was examined in macrophages isolated from the blood of 6 different human volunteers, 5 of whom are Caucasians less than 36 years old, and one of whom is a 25 year old African American woman. The methods described above for the analysis of mouse macrophage intoxication were employed. None of the individuals surveyed had LeTx-susceptible macrophages. Contemporaneously-assayed mouse macrophage controls behaved as expected, indicating that the intoxication protocols were successful. In addition, the human macrophages were all susceptible to LFn-DTA, which is a fusion protein that contains the N-terminal anthrax lethal factor sequences required for protective-antigen-mediated cell entry and the catalytic domain of diphtheria toxin. This result suggests that the cells we obtained had intact mechanisms for the uptake of native LeTx.

Different Macrophage Differentiation Protocols

To address the issue that only macrophages susceptible to LeTx-mediated cytolysis were observed in these experiments, we tested whether our differentiation protocols failed to yield the appropriate cell type. The methods employed utilized Ficoll gradient isolation of the mononuclear cell fraction, followed by selection for adherence on tissue culture plastic, and a week-long differentiation of the cells in the presence of a variety of different medias. Attempts were made to grow the adherent mononuclear cells in RPMI supplemented with 10% FCS, with 10% FCS+5% human donor serum, with 20% FCS and 30% L-cell conditioned medium (a source of M-CSF that we use for our mouse bone marrow derived macrophages), and with 10% FCS plus either recombinant human M-CSF or human GM-CSF. While the yields of the cells varied widely depending on the media (with recombinant M-CSF and GM-CSF resulting in the highest yields), the phenotypic result from a given individual was always the same. FACs analysis of the resulting cells demonstrated that approx 98% were positive for CD14, CD11b, or both, suggesting that we were obtaining the correct cells.

b. Genetic screening for susceptibility or resistance to anthrax infection:

In these methods, subjects are selected as described above, and tested for the identity of allelic variants of KiflC or other genes identified according to the invention. Comparison of the observed KiflC genotype with those found to associate with anthrax resistance or susceptibility, as determined in the above Examples, permits predictive determination of the test subject's resistance or susceptibility to anthrax infection or its effects.

The polynucleotides encoding KiflC and allelic variants thereof may be used for diagnostic purposes. A variety of protocols for measuring KiflC levels; including ELISAs, RIAs, and FACS, are known in the art and provide a basis for detecting KiflC or its encoded product.

Other diagnostic methods include use of polynucleotides in a variety of methods. The polynucleotides which may be used include oligonucleotide sequences, complementary RNA and DNA molecules, and PNAs. The polynucleotides may be used to detect and quantitate gene expression in biopsied tissues in which expression of KiflC may be correlated with susceptibility to the lethal effects of LeTX. The diagnostic assay may be used to determine absence, presence, and excess expression of KiflC, and to monitor regulation of KiflC levels during therapeutic intervention.

One suitable method for diagnosis includes contacting a test sample with the KiflC gene, an allele or fragment thereof, or expression product of the KiflC gene, an allele or fragment thereof; and detecting interaction of the test sample with the KiflC gene, an allele or fragment thereof, or expression product of the KiflC gene, an allele or fragment thereof. The test sample is a mammalian tissue or fluid (e.g. blood) sample. The KiflC gene, an allele or fragment thereof, or expression product of the KiflC gene, an allele or fragment thereof suitably can be detectably labeled e.g. with a fluorescent or radioactive component.

In one aspect, hybridization with oligonucleotide probes that are capable of detecting polynucleotide sequences, including genomic sequences, encoding KiflC or closely related molecules may be used to identify nucleic acid sequences which encode KiflC. The specificity of the probe, whether it is made from a highly specific region, e.g., the 5′ regulatory region, or from a less specific region, e.g., a conserved motif, and the stringency of the hybridization or amplification (maximal, high, intermediate, or low), will determine whether the probe identifies only naturally occurring sequences encoding KiflC, allelic variants, or related sequences.

Probes may also be used for the detection of related sequences, and should preferably have at least 50% sequence identity or homology to any of the KiflC encoding sequences, more preferably at least about 60, 70, 75, 80, 85, 90 or 95 percent sequence identity to any of the KiflC encoding sequences (sequence identity determinations discussed above, including use of BLAST program). The hybridization probes of the subject invention may be DNA or RNA and may be derived from the sequences of the invention or from genomic sequences including promoters, enhancers, and introns of the KiflC gene.

“Homologous”, as used herein, refers to the subunit sequence similarity between two polymeric molecules, e.g., between two nucleic acid molecules such as two DNA molecules, or two polypeptide molecules. When a subunit position in both of the two molecules is occupied by the same monomeric subunit (e.g., if a position in each of two DNA molecules is occupied by adenine) then they are homologous at that position. The homology between two sequences is a direct function of the number of matching or homologous positions. For example, if 5 of 10 positions in two compound sequences are matched or homologous then the two sequences are 50% homologous, if 9 of 10 are matched or homologous, the two sequences share 90% homology. By way of example, the DNA sequences 3′ ATTGCC 5′ and 3′ TTTCCG 5′ share 50% homology.

Means for producing specific hybridization probes for DNAs encoding KiflC include the cloning of polynucleotide sequences encoding KiflC or KiflC derivatives into vectors for the production of mRNA probes. Such vectors are known in the art, are commercially available, and may be used to synthesize RNA probes in vitro by means of the addition of the appropriate RNA polymerases and the appropriate labeled nucleotides. Hybridization probes may be labeled by a variety of reporter groups, for example, by radionuclides such as ³²P or ³²S, or by enzymatic labels, such as alkaline phosphatase coupled to the probe via avidin-biotin coupling systems, fluorescent labeling, and the like.

The polynucleotide sequences encoding KiflC may be used in Southern or Northern analysis, dot blot, or other membrane-based technologies; in PCR technologies; in dipstick, pin, and multiformat ELISA-like assays; and in microarrays utilizing fluids or tissues from patients to detect altered KiflC expression. Gel-based mobility-shift analyses may be employed. Other suitable qualitative or quantitative methods are well known in the art.

Identity of genes, or variants thereof, can be verified using techniques well known in the art. Examples include but are not limited to, nucleic acid sequencing of amplified genes, hybridization techniques such as single nucleic acid polymorphism analysis (SNP), microarrays wherein the molecule of interest is immobilized on a biochip. Overlapping cDNA clones can be sequenced by the dideoxy chain reaction using fluorescent dye terminators and an ABI sequencer (Applied Biosystems, Foster City, Calif.) Any type of assay wherein one component is immobilized may be carried out using the substrate platforms of the invention. Bioassays utilizing an immobilized component are well known in the art. Examples of assays utilizing an immobilized component include for example, immunoassays, analysis of protein-protein interactions, analysis of protein-nucleic acid interactions, analysis of nucleic acid-nucleic acid interactions, receptor binding assays, enzyme assays, phosphorylation assays, diagnostic assays for determination of disease state, genetic profiling for drug compatibility analysis, SNP detection, etc.

A resistant or susceptibility gene means the gene and all currently known variants thereof, including the different mRNA transcripts to which the gene and its variants can give rise, and any further gene variants which may be elucidated. In general, however, such variants will have significant homology (sequence identity) to a sequence of KiflC (resistant or susceptible), e.g. a variant will have at least about 70 percent homology (sequence identity) to a sequence of KiflC, more typically at least about 75, 80, 85, 90, 95, 97, 98 or 99 homology (sequence identity) to a sequence of KiflC. Homology of a variant can be determined by any of a number of standard techniques such as a BLAST program.

Identification of a nucleic acid sequence capable of binding to a biomolecule of interest can be achieved by immobilizing a library of nucleic acids onto the substrate surface so that each unique nucleic acid was located at a defined position to form an array. The array would then be exposed to the biomolecule under conditions which favored binding of the biomolecule to the nucleic acids. Non-specifically binding biomolecules could be washed away using mild to stringent buffer conditions depending on the level of specificity of binding desired. The nucleic acid array would then be analyzed to determine which nucleic acid sequences bound to the biomolecule. Preferably the biomolecules would carry a fluorescent tag for use in detection of the location of the bound nucleic acids.

An assay using an immobilized array of nucleic acid sequences may be used for determining the sequence of an unknown nucleic acid; single nucleotide polymorphism (SNP) analysis; analysis of gene expression patterns from a particular species, tissue, cell type, etc.; gene identification; etc.

Additional diagnostic uses for oligonucleotides designed from the sequences encoding KiflC may involve the use of PCR. These oligomers may be chemically synthesized, generated enzymatically, or produced in vitro. Oligomers will preferably contain a fragment of a polynucleotide encoding KiflC, or a fragment of a polynucleotide complementary to the polynucleotide encoding KiflC, and will be employed under optimized conditions for identification of a specific gene. Oligomers may also be employed under less stringent conditions for detection or quantitation of closely-related DNA or RNA sequences.

High stringency conditions are known in the art; see for example Maniatis et al., Molecular Cloning: A Laboratory Manual, 2d Edition, 1989, and Short Protocols in Molecular Biology, ed. Ausubel, et al., both of which are hereby incorporated by reference. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Acid Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH and nucleic acid concentration) at which 50% of a nucleic acid sequence complementary to the target, hybridizes to the target sequence at equilibrium. Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short nucleic acid sequences (e.g. 10 to 50 nucleotides) and at least about 60° C. for long nucleic acid sequences (e.g. greater than 50 nucleotides). Stringent conditions may also be achieved with the addition of destabilizing agents such as formamide.

The phrase “stringent hybridization” is used herein to refer to conditions under which polynucleic acid hybrids are stable. As known to those of skill in the art, the stability of hybrids is reflected in the melting temperature (Tₘ) of the hybrids. In general, the stability of a hybrid is a function of sodium ion concentration and temperature. Typically, the hybridization reaction is performed under conditions of lower stringency, followed by washes of varying, but higher, stringency. Reference to hybridization stringency relates to such washing conditions. As used herein, the phrase “moderately stringent hybridization” refers to conditions that permit target-DNA to bind a complementary nucleic acid that has about 60% identity, preferably about 75% identity, more preferably about 85% identity to the target DNA; with greater than about 90% identity to target-DNA being especially preferred. Preferably, moderately stringent conditions are conditions equivalent to hybridization in 50% formamide, 5×Denhart's solution, 5×SSPE, 0.2% SDS at 42° C., followed by washing in 0.2×SSPE, 0.2% SDS, at 65° C.

The phrase “high stringency hybridization” refers to conditions that permit hybridization of only those nucleic acid sequences that form stable hybrids in, for example, 0.018 M NaCl at 65° C. (i.e., if a hybrid is not stable in 0.018 M NaCl at 65° C., it will not be stable under high stringency conditions). High stringency conditions can be provided, for example, by hybridization in 50% formamide, 5×Denhart's solution, 5×SSPE, 0.2% SDS at 42° C., followed by washing in 0.1×SSPE, and 0.1% SDS at 65° C.

The phrase “low stringency hybridization” refers to conditions equivalent to hybridization in 10% formamide, 5×Denhart's solution, 6×SSPE, 0.2% SDS at 42° C., followed by washing in 1×SSPE, 0.2% SDS, at 50° C. Denhart's solution and SSPE (see, e.g., Sambrook et al., Molecular Cloning, A Laboratory Manual, Cold Spring Harbor Laboratory Press, 1989) are well known to those of skill in the art as are other suitable hybridization buffers.

In further embodiments, oligonucleotides or longer fragments derived from any of the mouse or human polynucleotide sequences described herein, for example SEQ ID NOs: 1-23, may be used as targets in a microarray. The microarray can be used to monitor the identity and/or expression level of large numbers of genes and gene transcripts simultaneously to identify genes with which KiflC or its product interacts and/or to assess the efficacy of candidate therapeutic agents in regulating genes that mediate anthrax susceptibility. Microarrays may be used to particular advantage in diagnostic assays, to identify genetic variants, mutations, and polymorphisms of genes that mediate anthrax susceptibility in a biological sample from a mammal, such as a human or other research subject or clinical patient. This information may be used to determine gene function, to understand the genetic basis of anthrax resistance or sensitivity, to determine a subject's relative resistance or susceptibility to anthrax, and to develop and monitor the activities of therapeutic agents.

In other embodiments, oligonucleotides or longer fragments derived from any of the polynucleotide sequences herein (including, in non-limiting fashion, human sequences) and genomic sequences adjacent to them may be used as diagnostic reagents, such as to detect single-nucleotide polymorphisms or other variations or mutations in KiflC or a homologous gene, amplification of KiflC or homologous nucleic acid sequences, and for use in nucleic acid sequencing methods.

Microarrays may be prepared, used, and analyzed using methods known in the art (see, e.g., Brennan et al., 1995, U.S. Pat. No. 5,474,796; Schena et al., 1996, Proc. Natl. Acad. Sci. U.S.A. 93: 10614-10619; Baldeschweiler et al., 1995, PCT application WO95/251116; Shalon, et al., 1995, PCT application WO95/35505; Heller et al., 1997, Proc. Natl. Acad. Sci. U.S.A. 94: 2150-2155; and Heller et al., 1997, U.S. Pat. No. 5,605,662).

Expression or activity levels for KiflC also may be examined. Normal or standard values for KiflC expression are established by combining body fluids or cell extracts taken from normal mammalian subjects, preferably human, with antibody to KiflC under conditions suitable for complex formation. The amount of standard complex formation may be quantitated by various methods, preferably by photometric means. Quantities of KiflC expressed in subject, control, and disease samples from biopsied tissues are compared with the standard values. Deviation between standard and subject values establishes the parameters for susceptibility or resistance to the lethal effects of LeTx. Parameters studied include, but are not limited to, the below and those described throughout the specification:

Antisera to Ltxsl that can be used to determine the subcellular localization of the protein. Without being bound by theory, the LeTx susceptibility-inducing mutations in KiflC could affect the steady-state levels of the encoded protein, either by affecting synthesis or protein turnover. The preferred means is to utilize the antisera against peptides of KiflC. These antisera can be used to probe Western blots of protein lysates obtained from mouse bone-marrow derived macrophages that have different variants of KiflC. For this purpose, mouse strains are evaluated representative of each of the variants we have detected: C57BL/6J (common resistant allele), C3H/HeJ (common susceptible allele), CAST (another susceptibility allele), and DBA/2J (another resistance allele). Since the KiflC RNA levels of these strains are equivalent, any reproducible differences in the intensity of the KiflC band (normalized for total protein and expression of tubulin, or some other common control antigen) can be considered to reflect differences in the steady state levels of the protein.

Again without being bound by theory, one or more of the polymorphic forms of KiflC may be proteolytically digested by LF. To search for evidence of proteolysis of KiflC, and to determine if differences in susceptibility to digestion exist among the susceptible and resistant forms of KiflC, the above methods are suitably be used. The KiflC antisera can be employed to probe Western blots of proteins obtained from mouse macrophages isolated from the strains described above. In addition to the samples from unintoxicated macrophages, lanes also can be included with proteins isolated from macrophages that have been intoxicated with LeTx. The intoxicated samples are examined for evidence of alterations in relative KiflC band intensity (again, normalized to total protein and tubulin) and/or migration (which would be altered if the protein is clipped into smaller fragments, as is observed with the MAPKKs cleaved by LF).

The choices of toxin amount and length of incubation for this experiment are discernable, e.g. the proteolysis of MAPKK3 can be readily detected in intoxicated resistant and susceptible macrophages (Watters et al 2001, Genomics 73: 223-231). Methods to achieve synchronous, high-level intoxication, are below.

KiflC Phosphorylation

Endogenous KiflC is phosphorylated on both tyrosine and serine residues, and that the phosphorylation of a particular serine (at position 1092 in the human sequence, 1089 in the mouse) is important in the ability of KiflC to bind to 14-3-3 adaptor proteins. The level and location of phosphorylation events of the proteins encoded by the different KiflC variants can be investigated.

Immunoprecipitation is used to bring down either overexpressed or endogenous KiflC protein from cell lysates. The KiflC antisera and standard methods can be utilized to perform simple experiments of this type, then blot and probe the immunoprecipitated KiflC protein with commercially available anti-phosphotyrosine antibody (the results can be normalized by reprobing the blot with the KiflC antisera). Using this method, reasonably quantitative information can be obtained about the relative levels of tyrosine phosphorylation of the different KiflC variants both before and after LeTx intoxication. The serine phosphorylation status also can be measured using methods well known in the art. Immunoprecipitated protein can be employed in mass spectroscopy experiments to determine the relative amount and location of phosphorylation events throughout the protein. It can be important to do these experiments on cells having the different polymorphic forms of KiflC. In addition, the LeTx-intoxicated and unintoxicated cells can be examined to search for alterations in the amount, location, and kind of KiflC phosphorylation.

Observation of different patterns or quantities of phosphorylation, either in normal or LeTx-intoxicated conditions, can provide an indication clue about the effects of the susceptibility inducing mutations. Without being to be bound by theory, it would indicate that the different forms of KiflC are differentially able to receive signals transduced to them by regulatory kinases or phosphatases. Certainly, in combination with an observation of altered cellular localization of the different protein forms it would indicate that the regulation of KiflC activity is affected by signalling pathways that could be directly or indirectly affected by LeTx. Known candidate kinases and phosphatases for differential interaction with the polymorphic KiflC proteins also can be used. Screening for physical interactions between Ltxsl and other proteins.

KiflC can interact with several known proteins in cells. These proteins include members of the 14-3-3 adaptor family (which could play an important role in cargo binding) and KiflC itself (which could play an important role in regulating KiflC's motor activity). Since several of the susceptibility-inducing polymorphisms that we observe occur in regions hypothesized to be involved in interactions with other proteins, the capacity of the polymorphic forms of KiflC to interact with these candidate interacting proteins (or candidate drug compounds, see Example 4) can be analyzed.

Basically, human KIFl C interacts with some, but not all, 14-3-3 proteins, and in which they provide evidence of KIFl C homo-dimer formation (Dorner et al 1999). KIFl C physically interacts with 14-3-3 beta, gamma, epsilon, and zeta, but not eta, sigma, or tau. Furthermore, 14-3-3 gamma can be co-immunoprecipitated by pulling down KIFl C, and vice versa, and co-immunoprecipitate native KiflC with tagged KiflC. Dorner et al indicated that treatment of non-transfected cell lysates with the crosslinking reagent DFNDB (an aryl hydride that reacts to primary amines, thiol groups, and phenolate groups) allowed the observation of a KiflC antibody reactive western band with a larger molecular weight that was consistent with the size of the homodimer.

These experiments with mouse macrophages of different KiflC genotype (that are either intoxicated or not), using our new KiflC antisera and commercially available antibodies to various 14-3-3 proteins can be conducted. Differential affinity of the proteins are determined by quantitating the relative amounts of the interacting complexes (as observed by the co-immunoprecipitated 14-3-3 band, or by the larger KiflC reactive band in the cross linking experiment).

Use of epitope-tagged LF to determine the subcellular localization of LF in sensitive and resistant cells. The polymorphisms that are observed between the resistant and susceptible variants of KiflC could alter the ability of the protein to function as a molecular motor or in its ability to regulate this function properly. Each of these possible defects could result in differences in the overall intracellular distribution of the protein. Therefore, the steady state localization of the KiflC proteins encoded by the different susceptible and resistant variants can be analyzed.

The methods are straightforward in that standard immunofluorescence labeling techniques with the KiflC antisera can be employed. The intracellular localization can be examined of the endogenous protein contained in fixed (non-living) macrophages of the mouse strains described supra, looking for qualitative differences among the variants in the location, size, and distribution of compartments that contain KiflC.

To facilitate this analysis, double labeling of the cells can be undertaken with commercially available Golgi, ER, and other intracellular compartment marker antibodies, looking for differences in co-localization. The co-localization with suitable candidate cargo molecules also can be evaluated. For example, the previously observed connection between oxidative metabolite production and the sensitivity of macrophages to LeTx provides a likely avenue of pursuit. It has been previously shown that a portion of the cellular inducible nitric oxide synthase in macrophages is associated with the ER and with small punctate vesicles in the cytosol (Webb et al 2001). In addition, macrophages can assemble the NADPH oxidase complex onto the membranes of vesicles upon stimulation (Vazquez-Torres et al 2000, Science 287: 1655-1658). These vesicles that contain obvious producers of oxidative burst metabolism seem to contribute to the macrophage's ability to kill pathogens.

Cells can be labeled with a variety of commercially available antibodies against iNOS and against different components of the NADPH oxidase complex to determine if KiflC engages in trafficking of these effector molecules, and to look for differences in how the susceptible and resistant variants co-localize. Co-labelling can be performed with commercially available dyes that react with NO and/or superoxide to produce fluorescent compounds. Dyes such as 4-amino-5-methylamino-2′,7′-difluorescein (DAF, or its diacetate derivative) and 2′,7′-dichlorodihydrofluorescein (DCFH, or its diacetate derivative) are both non-fluorescent, membrane permeable dyes that are captured inside cells by reaction with esterases, and react with NO or superoxide to produce fluorescent compounds. These can be visualized by fluorescent microscopy, and have been used to study the intracellular location of NO production (Kobayashi et al 2000, Histochem. Cell. Biol. 113, 251-257).

While this work can be done static images of dynamic processes, a number of obvious perturbations to the cells can be performed, and the cells fixed and analyzed at various time points post-perturbation. One form of perturbation is the addition of LeTx. In this case, it may be necessary to achieve a high degree of synchrony for the treatment, otherwise, different cells will be at different points in the response, confusing the analysis. Since the major rate limiting step of LeTx entry is in the assembly of the PA and LF complex on the cellular receptors, this can be addressed by utilizing PA that has been “pre-nicked” with trypsin. Incubation of the nicked PA plus LF with cells at 4 degrees allows the assembly of the intact toxin complex on the surface of all the cells, but discourages internalization. Then, release of the cold block will permit toxin internalization at roughly the same time in all the cells (Beauregard et al 2000, Cell. Micorbiol. 2: 251-258).

Other kinds of perturbations would include the use of Brefeldin A (BFA) (which alters the cellular distribution of KiflC, and makes resistant macrophages become susceptible), or the use of microtubule destabilizing treatments (which also induce susceptibility in resistant cells). Using these methods, we can study the redistribution of different polymorphic forms of KiflC in response to addition and removal of these different perturbations.

Biochemical Evidence That LF can Modify the Protein Product of Ltxsl

We have differentiated embryonic stem (ES) cell lines into macrophages that behave well in a variety of our phenotypic assays. The functionality of the tagged proteins is achieved by studying the LeTx susceptibility of these ES cell derived macrophages. By administering the tagged proteins into ES cell-derived macrophages, we will also be able to study the features of the tagged proteins in the appropriate cell type.

An important issue in understanding the role of KiflC in resistance to LeTx lies in determining the cargo carried by the kinesin. In addition to the co-localization studies with a candidate cargo, the following will also be conducted. Methods for purifications of cargo vesicles attached to Kifl family members are known in the art.

“Immuno-isolation” of vesicles carried by Unc-104/Kifl kinesins has been carried out by others (e.g.: Okada et al., 1995, Cell, 81: 769-780). Partially-purified cargo vesicles that have kinesin still attached are immunoaffinity purified using beads conjugated with antibodies to kinesin. To isolate KiflC vesicles, cells (e.g., C57BL/6J macrophages) are homogenized (using a sonicator or a dounce homogenizer) in the presence of protease inhibitors. KiflC spins down with the “high-speed” microsomal membrane fraction (Nakajima et al 2002), so the vesicles can be partially purified with lower speed spins. For example, 30,000×g for 10 minutes leaves most of the cellular KiflC in solution, whereas 100,000×g for 30 minutes brought KiflC down with the microsomal vesicles (Nakajima et al 2002). The homogenate can be spun down at 30,000×g for 10 minutes, followed by recovery of the supernatant and the pellet, monitoring for the presence of KiflC in each fraction by Western, and then adsorbing the supernatant to beads conjugated with our KiflC antibody, wash, and then elute by boiling in SDS loading buffer. These purified vesicle fractions can then be loaded onto 1-D or 2-D polyacrylamide gels to resolve and observe the immuno-isolated proteins by Coomassie or silver stain. KiflC will be detected by Western as a control. Several isolates can be performed, on both unintoxicated and intoxicated cells, looking for consistency of the preparations.

Ultimately, all the proteins in the purified vesicles will be identified and/or to identify proteins whose abundance changes with LeTx intoxication. Depending on the complexity of the fractionated proteins that seen on the gels, different conditions may be used. For example, if the mixture is relatively uncomplicated, proteolytic digests of the entire set of partially purified proteins can be carried out, then subject them to tandem LC/mass spectroscopy. The sequences of individual peptides can be searched against the sequence databases to help determine the identity of proteins within the mixture. If the mixture is too complex for such an analysis, individual proteins can be eluted from the gel and mass spectroscopy utilized for protein identification. The most abundant protein species from the vesicles can be suitably focused on, and on the proteins whose abundance in the vesicles changes upon LeTx treatment.

Cargo molecules can be readily identified, particularly if the cargo is not too complex. Alternately, it may be the case that proteins, in response to LeTx treatment, suddenly associate with the vesicles carried by KiflC. In either case, the identity of these cargo molecules can provide more information about how KiflC mediates resistance mechanisms to LeTx and aid in the identification of screening for candidate drug compounds (see Example 4).

Besides protein cargoes which can be relevant to the effects that KiflC has on LeTx susceptibility, alternative molecules also can be studied such as carbohydrates, membrane components and the like. Without being to be bound by theory, an alternate explanation may be that KiflC is involved with recycling basic membrane elements of the secretion pathway.

Immunopurification of the vesicles using myc-tagged KiflC also can be performed, and the expression levels of the protein determined, since too high an overexpression may result in mislocalization of the protein.

### Example 4

Identification of Agents Useful in the Prevention or Treatment of Anthrax Infection and the Effects Thereof

As discussed above, the invention includes methods to identify agents of therapeutic interest, e.g. to identify compounds that have activity to reduce the adverse effects of LeTx. Candidate agents include numerous chemical classes, though typically they are organic compounds including small organic compounds, nucleic acids including oligonucleotides, and peptides. Small organic compounds suitably may have e.g. a molecular weight of more than about 40 or 50 yet less than about 2,500. Candidate agents may comprise functional chemical groups that interact with proteins and/or DNA.

Candidate agents may be obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of e.g. bacterial, fungal and animal extracts are available or readily produced.

Therapeutic agent assays of the invention suitably include, animal models, cell-based systems and non-cell based systems.

Preferably, KiflC, variants, fragments, or oligopeptides thereof are used for identifying agents of therapeutic interest, e.g. by screening libraries of compounds or otherwise identifying compounds of interest by any of a variety of drug screening or analysis techniques. The KiflC, allele, fragment, or oligopeptide thereof employed in such screening may be free in solution, affixed to a solid support, borne on a cell surface, or located intracellularly. The formation of binding complexes between KiflC and the agent being tested may be measured and then tested as described in the examples which follow whether there is an inhibitory effect on KiflC. Since inhibition of function of KiflC renders cells susceptible to LeTx, these agents or compounds would be deleterious. Compounds which do not inhibit KiflC are tested to determine whether they enhance protection of cells against LeTx.

Another technique for drug screening provides for high throughput screening of compounds having suitable binding affinity to the protein of interest (see, e.g., Geysen et al., 1984, PCT application WO84/03564). In this method, large numbers of different small test compounds are synthesized on a solid substrate. The test compounds are reacted with KiflC, or fragments thereof, and washed. Bound KiflC is then detected by methods well known in the art. Purified KiflC can also be coated directly onto plates for use in the aforementioned drug screening techniques. Alternatively, non-neutralizing antibodies can be used to capture the peptide and immobilize it on a solid support.

Other techniques for drug screening can be used such as for example, the methods described in Example 3 above.

### Example 5

Gene and/or Protein Therapy Using Nucleic Acid Sequences of Anthrax-Resistant KiflC Variants and/or Their Encoded Products

This example illustrates generally-useful methods by which to render macrophages from mammals susceptible to anthrax lethal toxin (LeTx) resistant to its lethal effects. For example, a nucleic acid sequence that codes for a C→T transition resulting in a proline to leucine substitution at position 578 renders macrophages susceptible to the lethal effects of anthrax toxin (LeTx). Therefore, a mutation at this position is determinative of susceptibility versus resistance to LeTx. Other important positions that are determinative of susceptibility versus resistance is a T→C transition at position 1027; an AG→TA nucleotide change at position 1066.

Ectopic Expression of a Resistance Allele of KiflC Partially Rescues Susceptible Macrophages

Our data indicate that perturbations of KiflC function cause macrophages to become susceptible to the cytolytic effects of LeTx. Previous observations, indicate that F1 hybrids between C57BL/6J and C3H/HeJ are completely susceptible to LeTx, even though they harbor one resistant KiflC allele (Watters and Dietrich, 2001, Genomics 73: 223-231). Together, these observations imply that the heterozygotes are susceptible due to haploinsufficiency of wild-type KiflC function. Under this model, any of the observed amino acid substitutions in the susceptible strains would impair the ability of KiflC to act properly as a molecular motor, and heterozygotes would have a level of KiflC function that is below a certain threshold required to maintain viability during LeTx challenge.

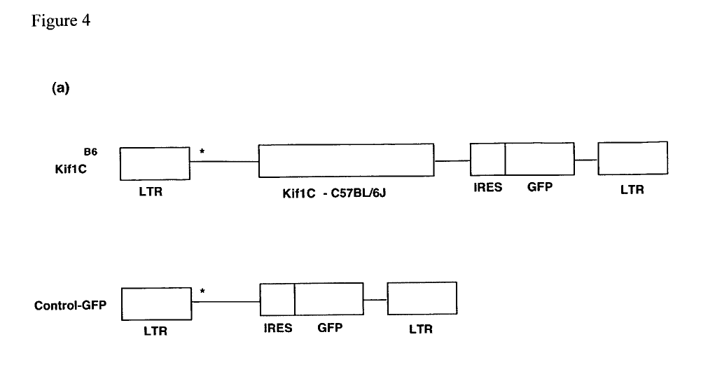

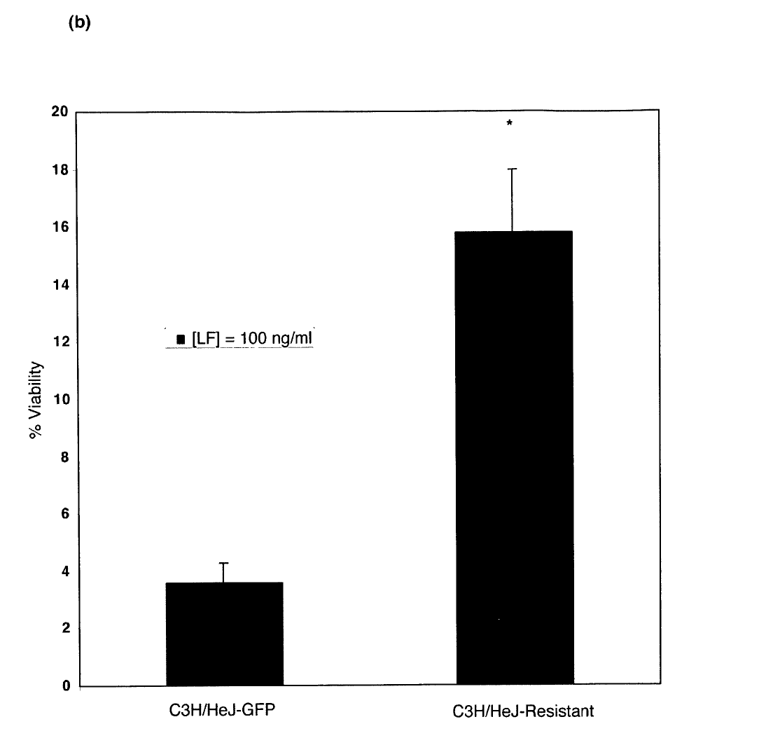

To test this directly, and to confirm the role of KiflC in resistance to LeTx, the resistant C57BL/6J allele of KiflC was transduced into susceptible C3H/HeJ macrophages using a retroviral vector derived from the Moloney Murine Leukemia Virus (MMLV) (FIG. 4A). As shown in FIG. 4B, expression of the C57BL/6J allele of KiflC resulted in a 4-fold increase in the proportion of C3H/HeJ macrophages that survived LeTx challenge. This incomplete rescue likely resulted from variable levels of expression of the construct in the primary cells, and that the cells that did not survive were expressing a level of KiflC^{B6} that is below the required resistance threshold. The reciprocal experiment was also performed, in which C57BL/6J macrophages transduced with the C3H/HeJ allele of KiflC were treated in an identical fashion, and these macrophages showed >95% viability in all fields analyzed.

The data are summarized in FIG. 4 which show that the expression of a resistance allele of KiflC partially rescues susceptible primary macrophages from LeTx-induced cytolysis. FIG. 4A presents a schematic representation of retroviral constructs containing the C57BL/6J allele of KiflC (KiflC^{B6}) or GFP only (control-GFP). The asterisk denotes the retroviral packaging signal. FIG. 4B shows rescue of susceptible macrophages. Susceptible primary macrophages were transduced with the control-GFP construct (C3H/HeJ-GFP) or the KiflC^{B6} construct (C3H/HeJ-Resistant), and viability in response to LeTx was determined. Data represents the average of 2 independent experiments±Standard Error of the Mean (SEM). The asterisk denotes a significant difference from control-GFP transduced cells, p<0.01.

Other Vectors and Nucleic Acid Molecules Useful in Gene Therapy According to the Invention, and Methods for Their Use

As discussed above, a preferred use of nucleic acid sequences (anthrax resistance genes) identified in the present invention, is for the generation of treatments that ameliorate adverse (toxic) effects of LeTx. The anthrax resistant genes can be expressed by a vector containing a DNA segment encoding the wild-type, alleles, variants, mutations or fragments of the genes. Mutations and alleles of the anthrax resistance genes are also preferably used in the construction of a vector for use in treatment. The vector comprising the desired nucleic acid sequence for conferring resistance to LeTx, preferably has at least one such nucleic acid sequence. Alternatively, the vector may be comprised of more than one such nucleic acid sequence, or combinations of allelic variants. The vector can also be comprised of cassettes of different allelic variants or wild type LeTx resistance genes.

According to the present invention, the coding sequence on the plasmid that encodes the anthrax lethal factor resistant genes is provided with a coding sequence that encodes an amino acid sequence whose presence on the protein results in a specific intracellular localization of the expressed protein. The nucleotide sequences that encode amino acid sequences which direct intracellular protein trafficking and which are included in the coding sequences of immunogenic proteins that are included in plasmid constructs used as DNA therapeutic compositions direct localization to specific areas in the cells which result in enhancement of resistance to anthrax toxin.

Introducing the genes, fragments or variants thereof, into an individual can include use of vectors, liposomes, naked DNA, adjuvant-assisted DNA, gene gun, catheters, etc. Vectors include chemical conjugates such as described in WO 93/04701, which has a targeting moiety (e.g. a ligand to a cellular surface receptor), and a nucleic acid binding moiety (e.g. polylysine), viral vector (e.g. a DNA or RNA viral vector), fusion proteins such as described in PCT/US95/02140 (WO 95/22618) which is a fusion protein containing a target moiety (e.g. an antibody specific for a target cell) and a nucleic acid binding moiety (e.g. a protamine), plasmids, phage etc. The vectors can be chromosomal, non-chromosomal or synthetic.

It is a preferred embodiment of this invention that the choice of cells for delivery of the anthrax resistance genes include embryonic stem cells, hematopoietic cells which can be differentiated into monocytes/macrophages using cytokines known in the art and delivery of these cells into an individual.

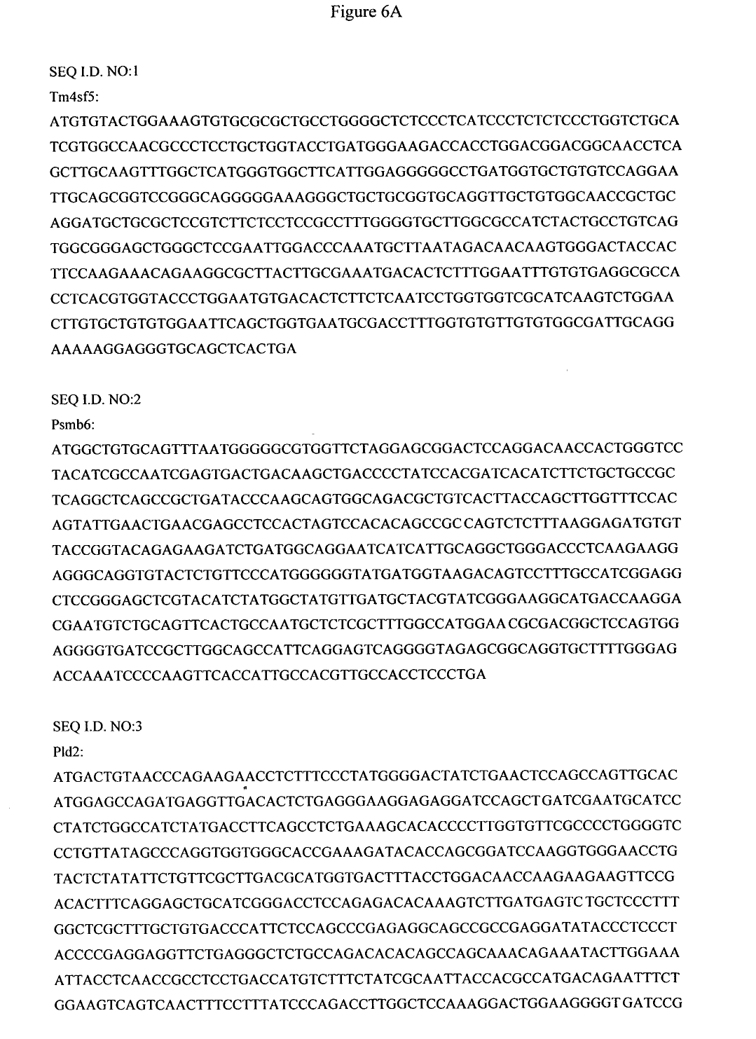

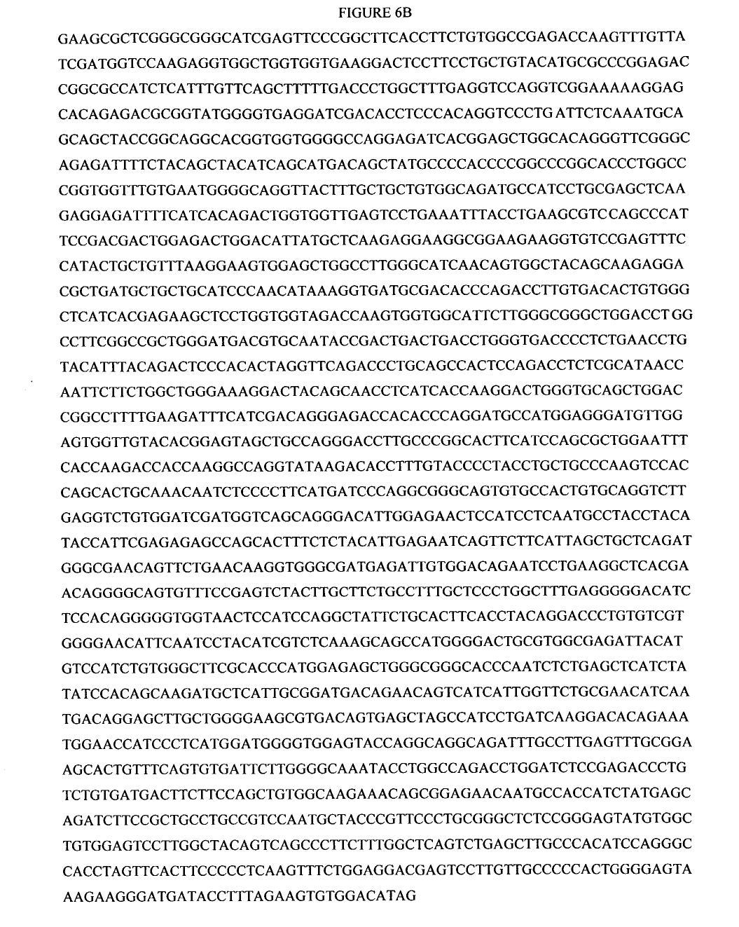

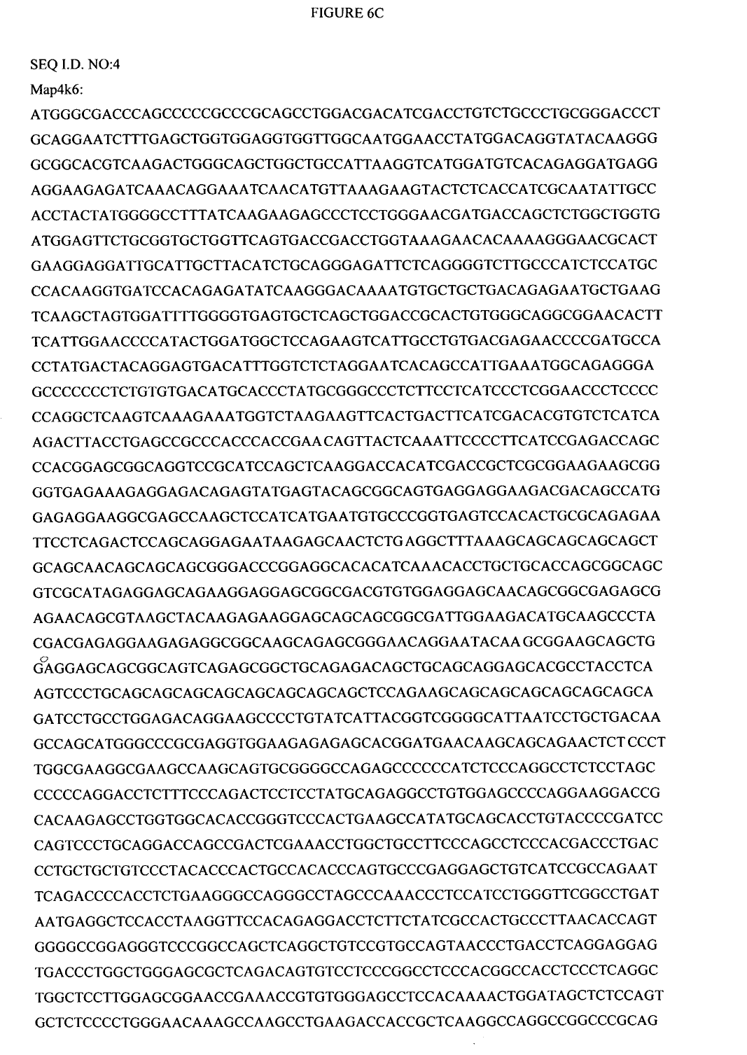

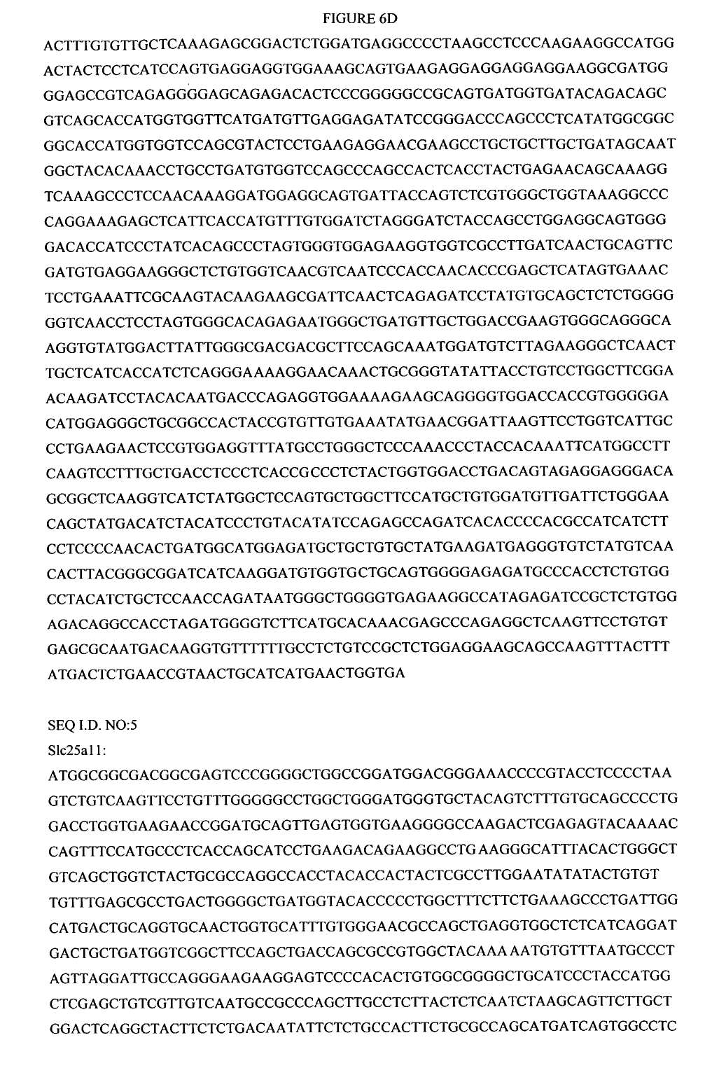

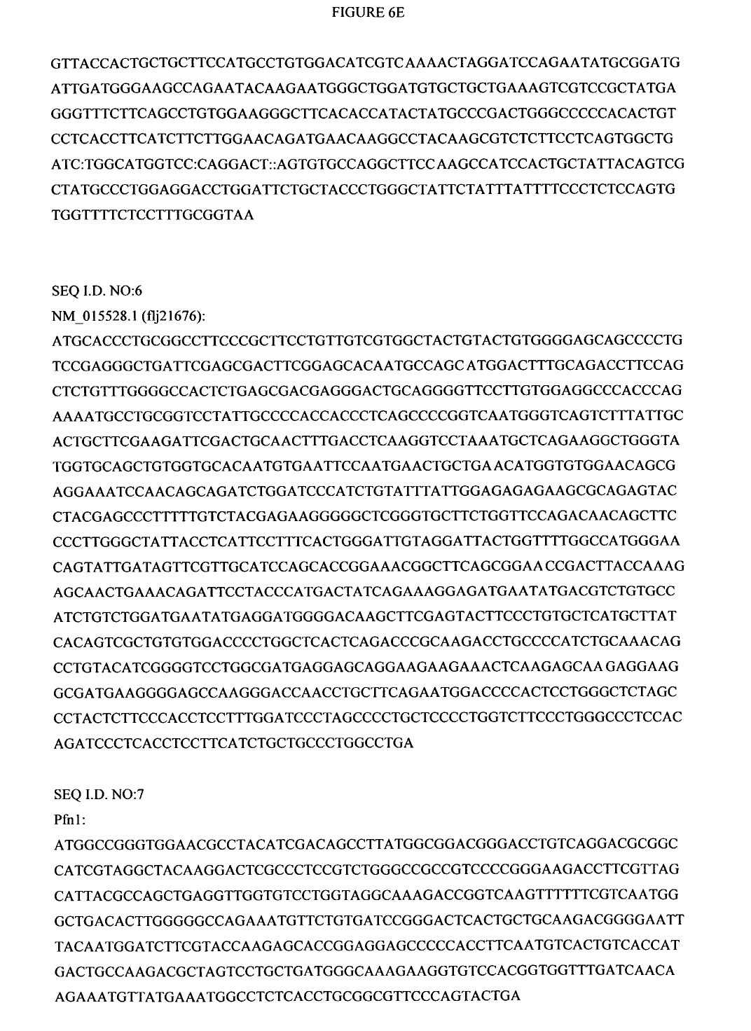

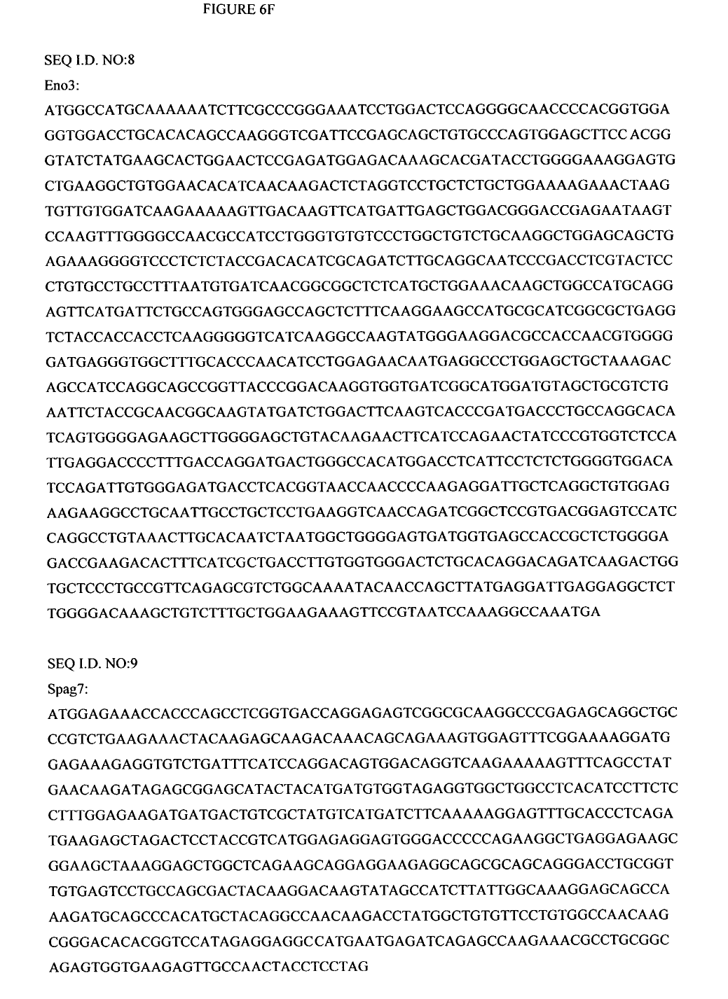

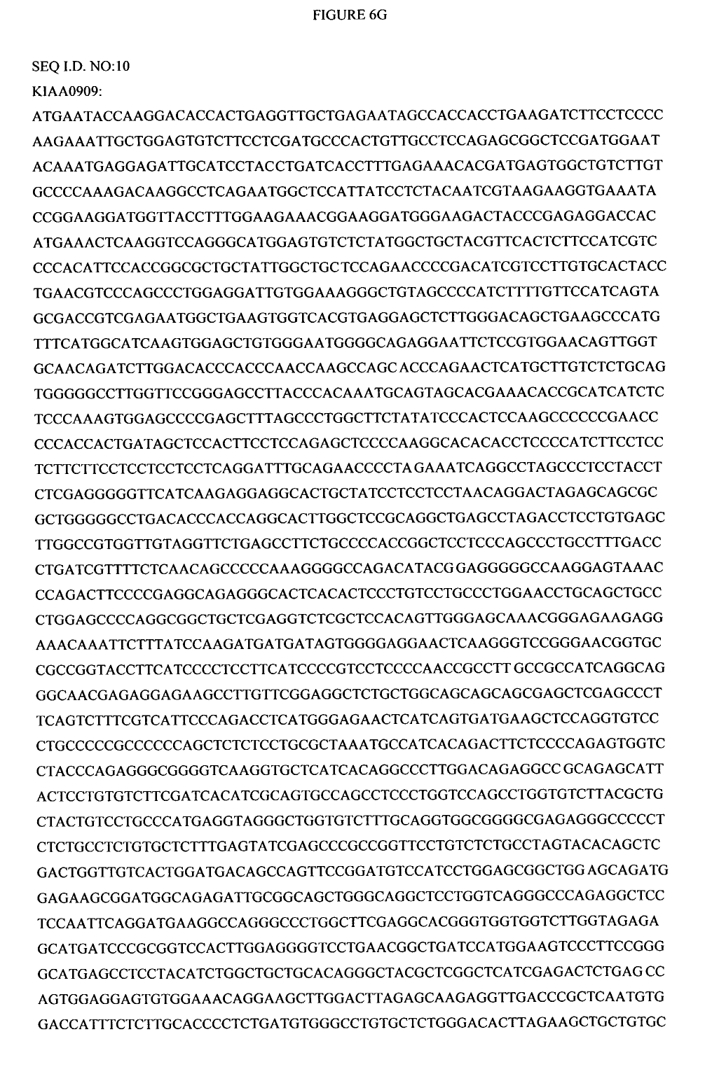

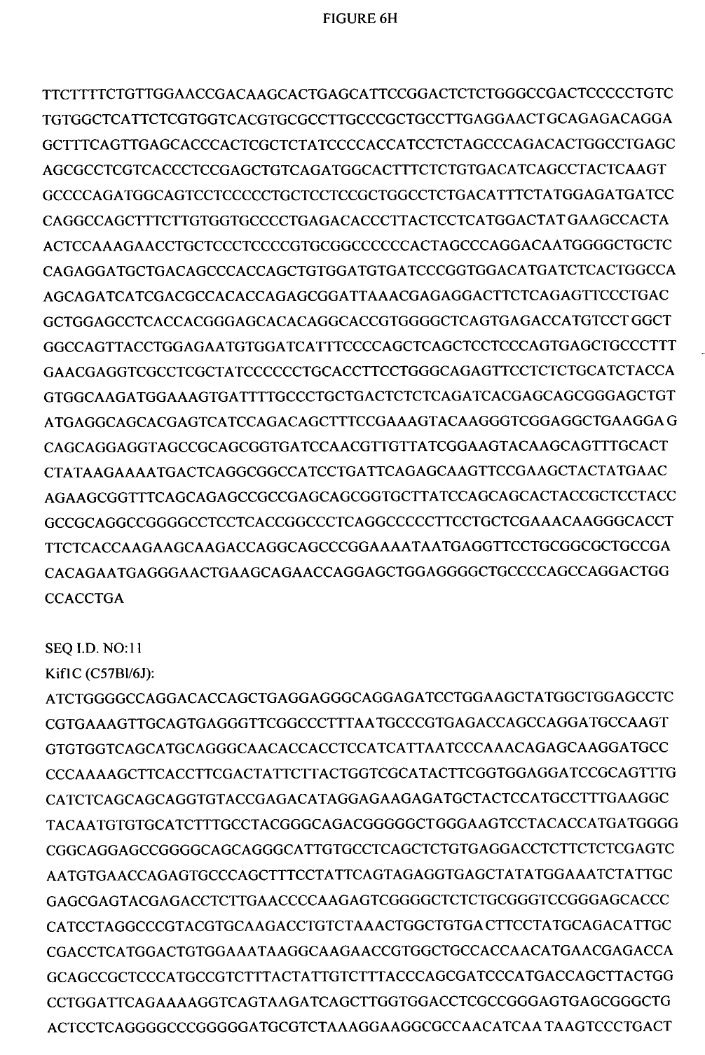

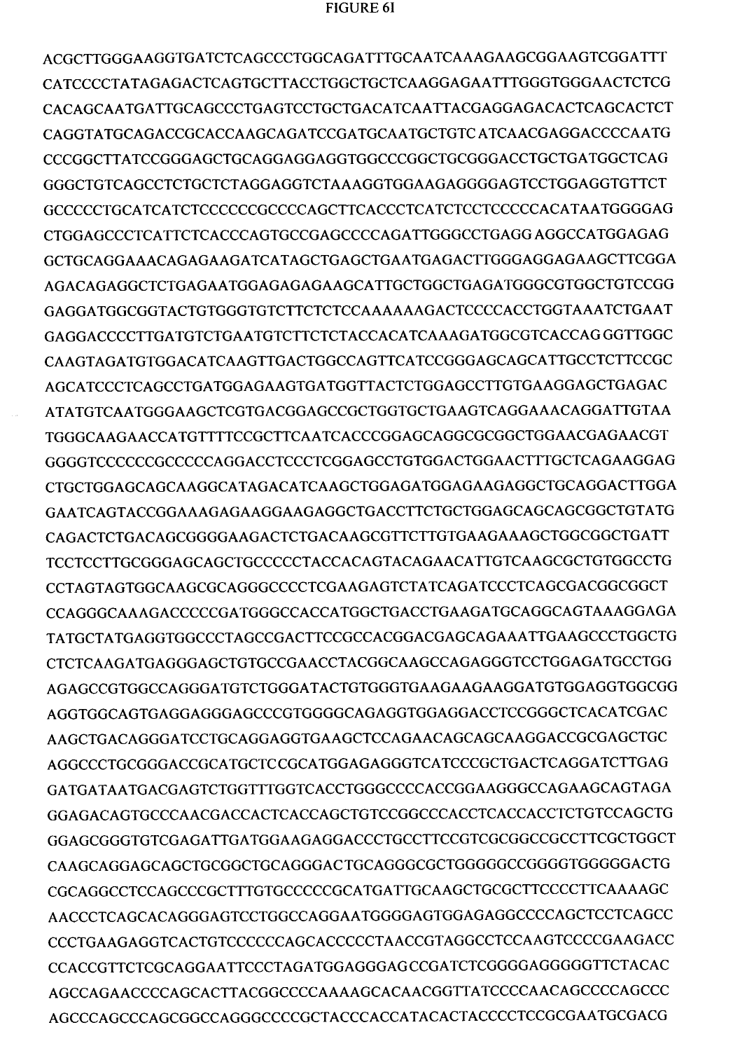

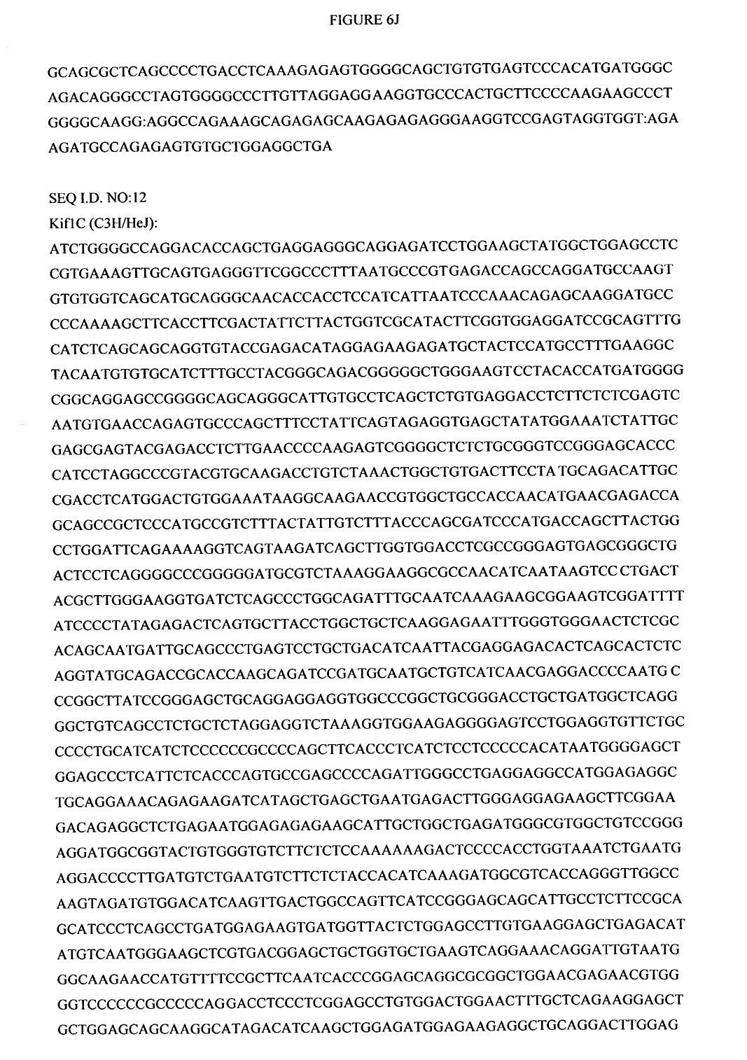

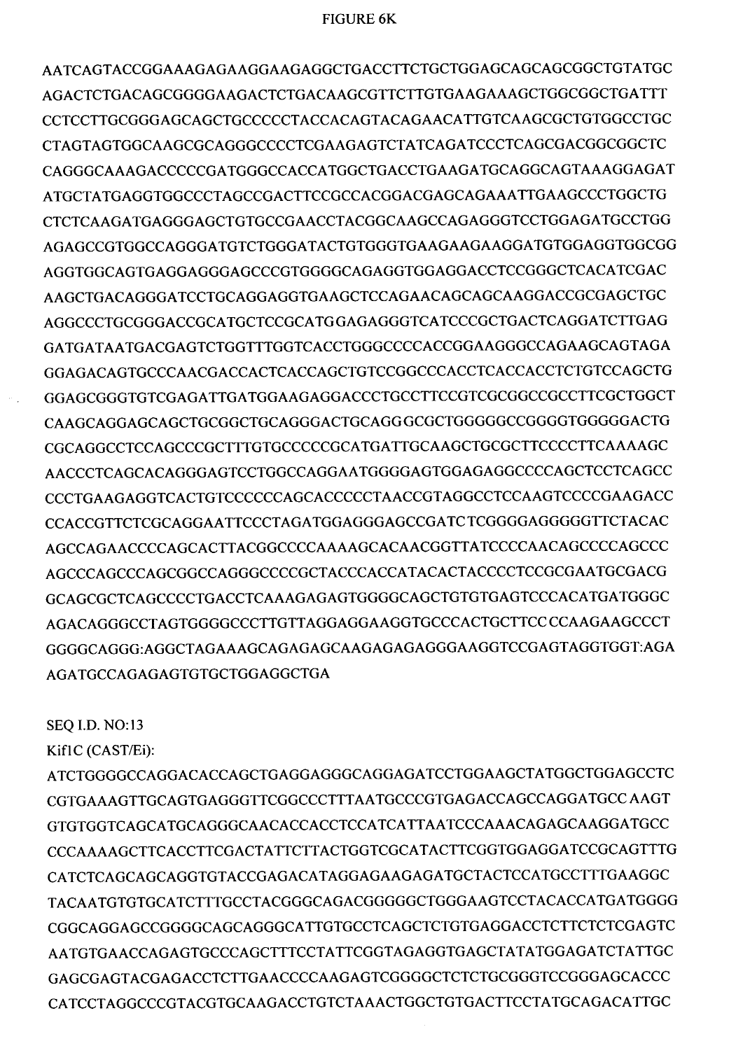

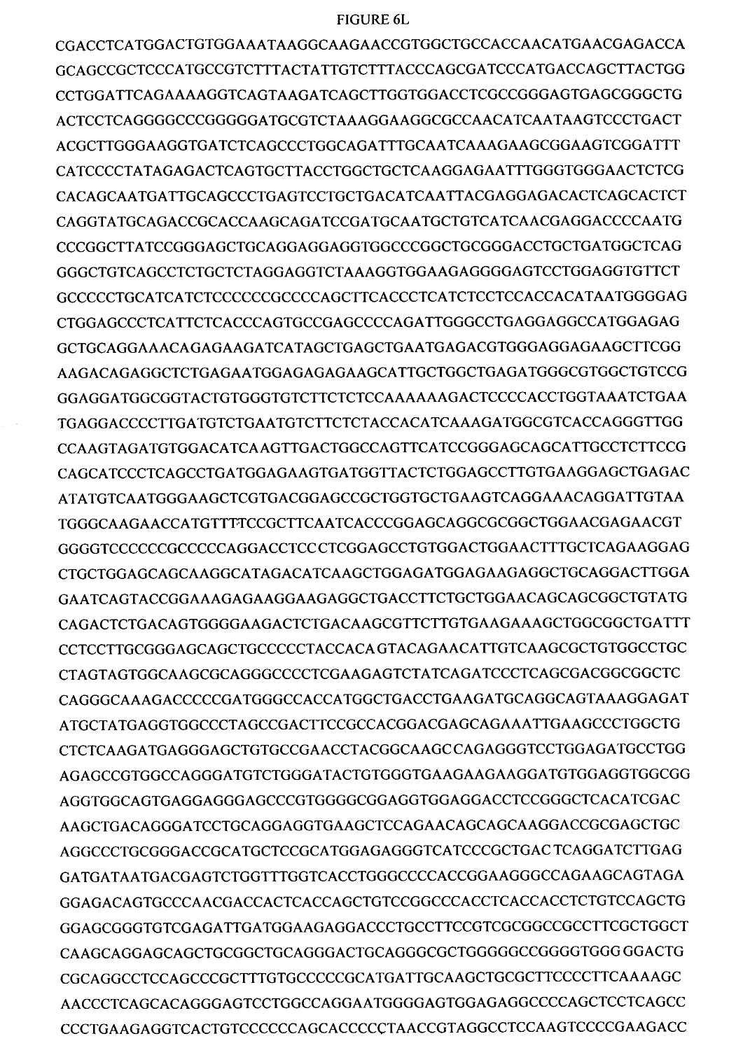

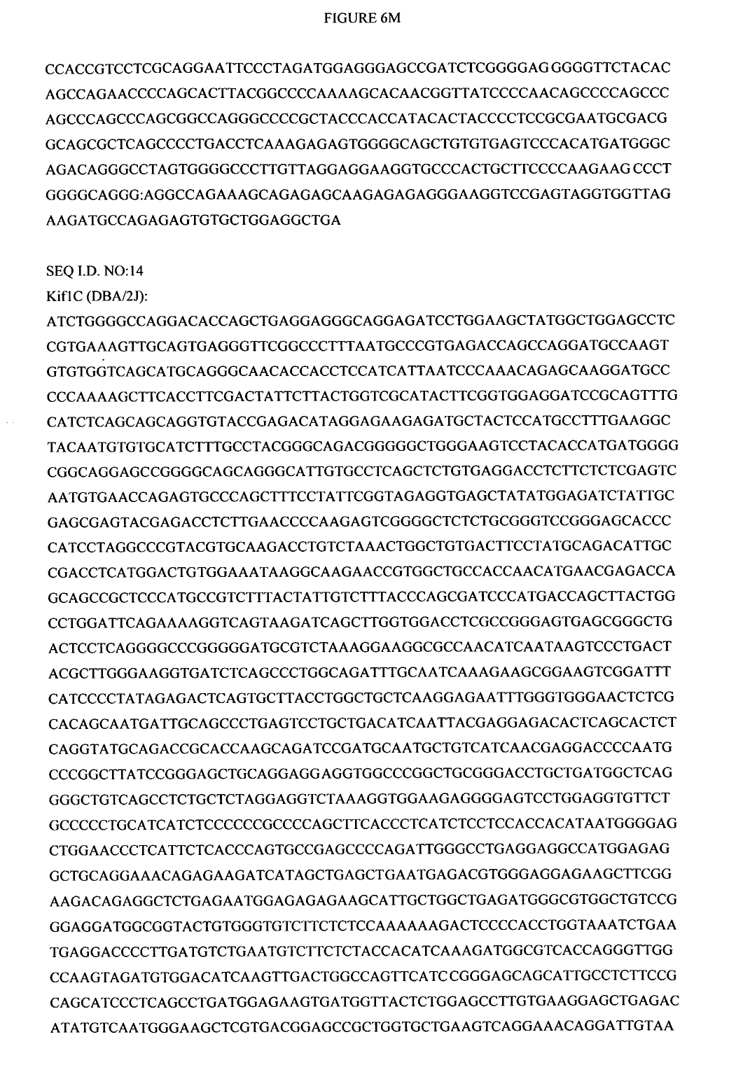

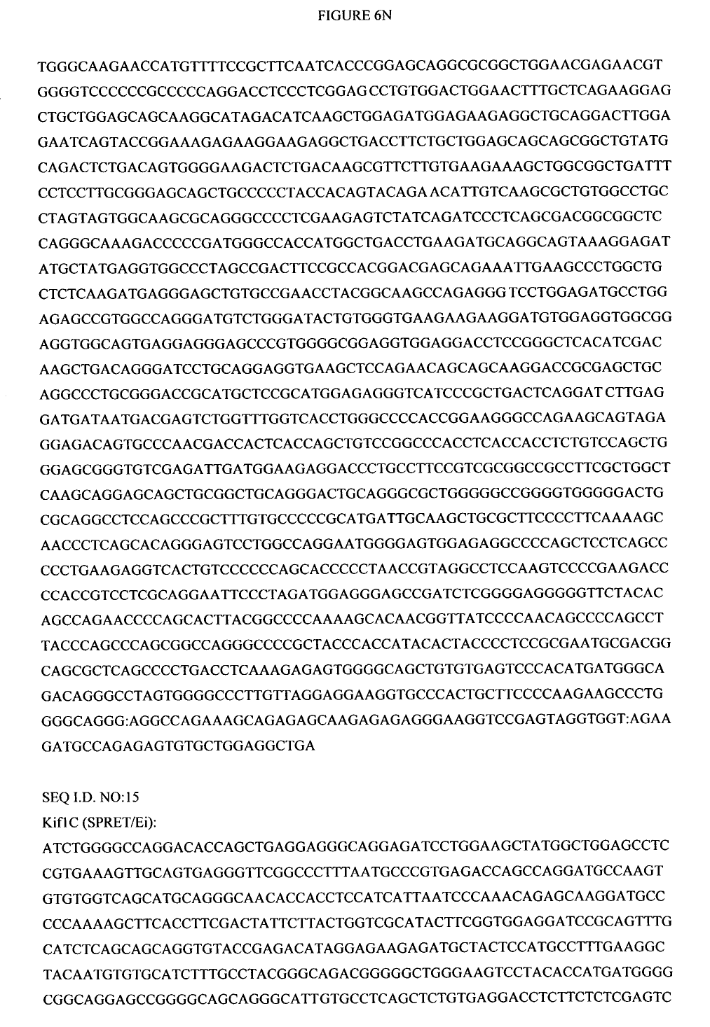

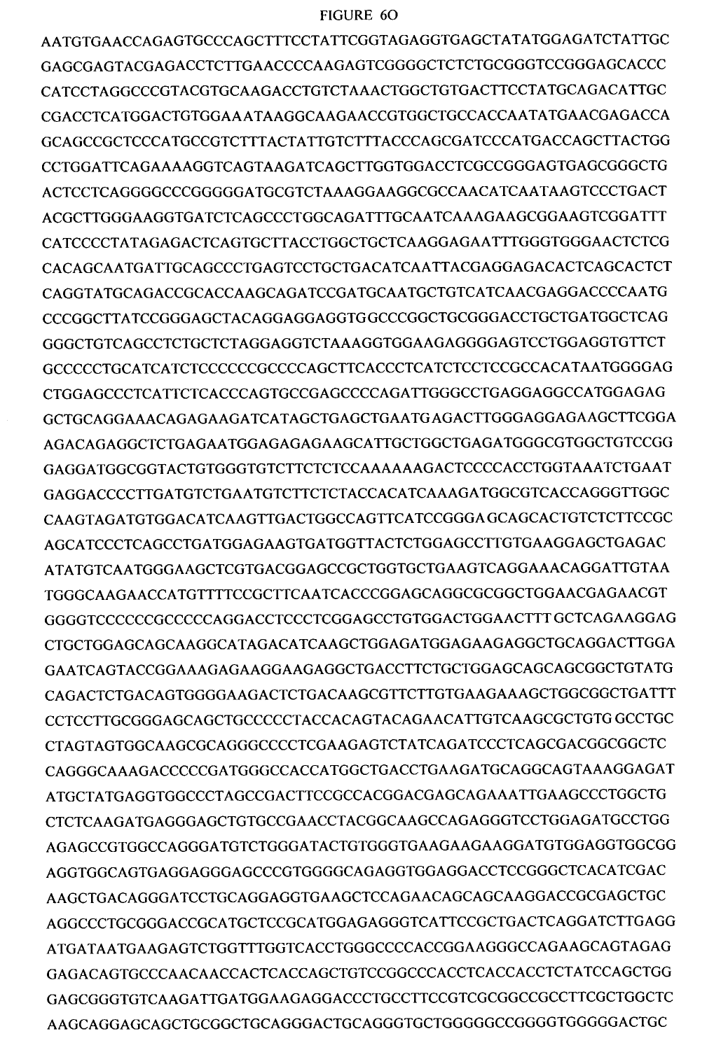

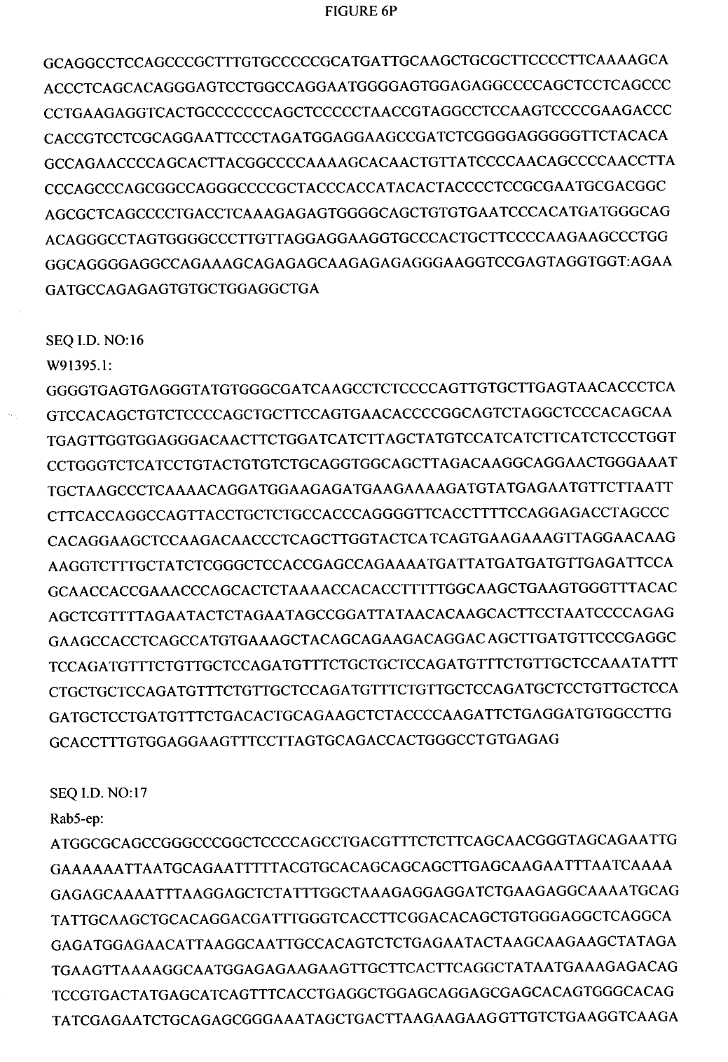

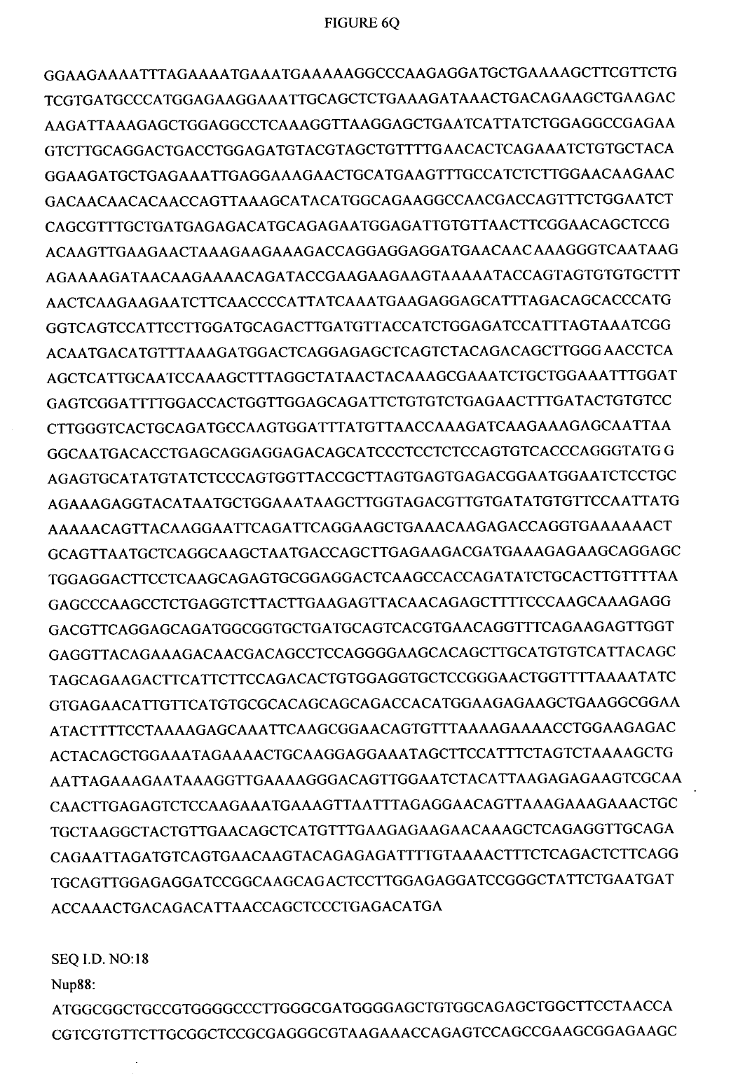

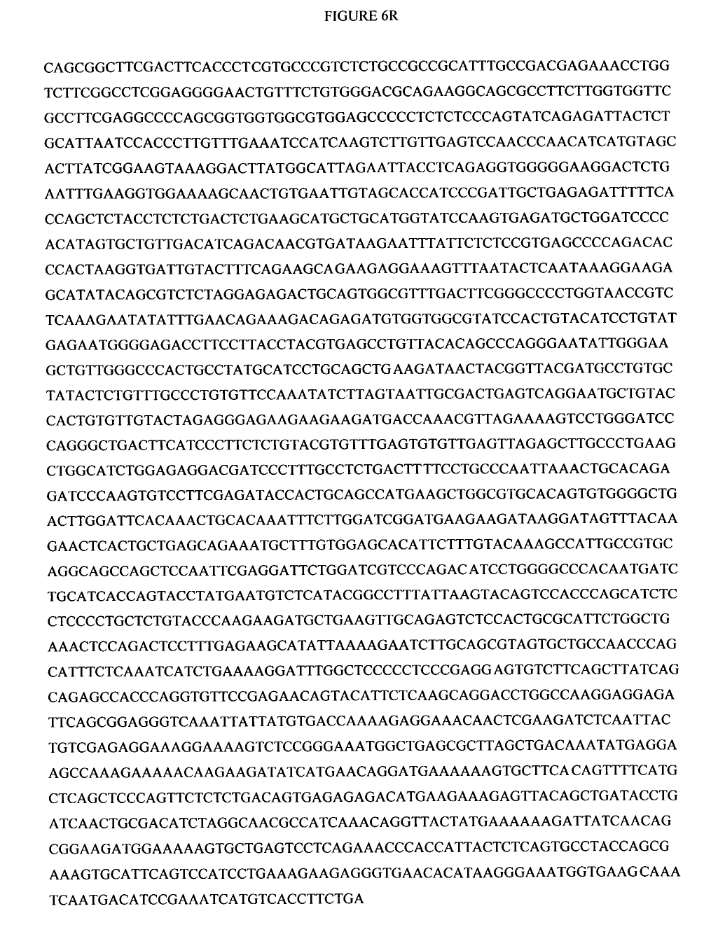

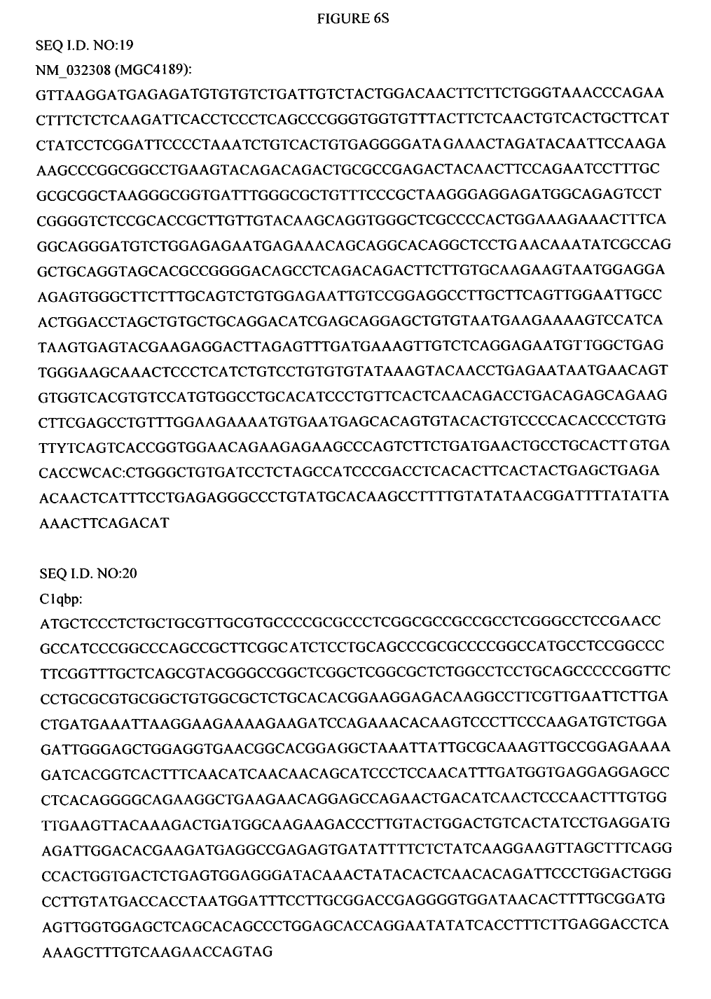

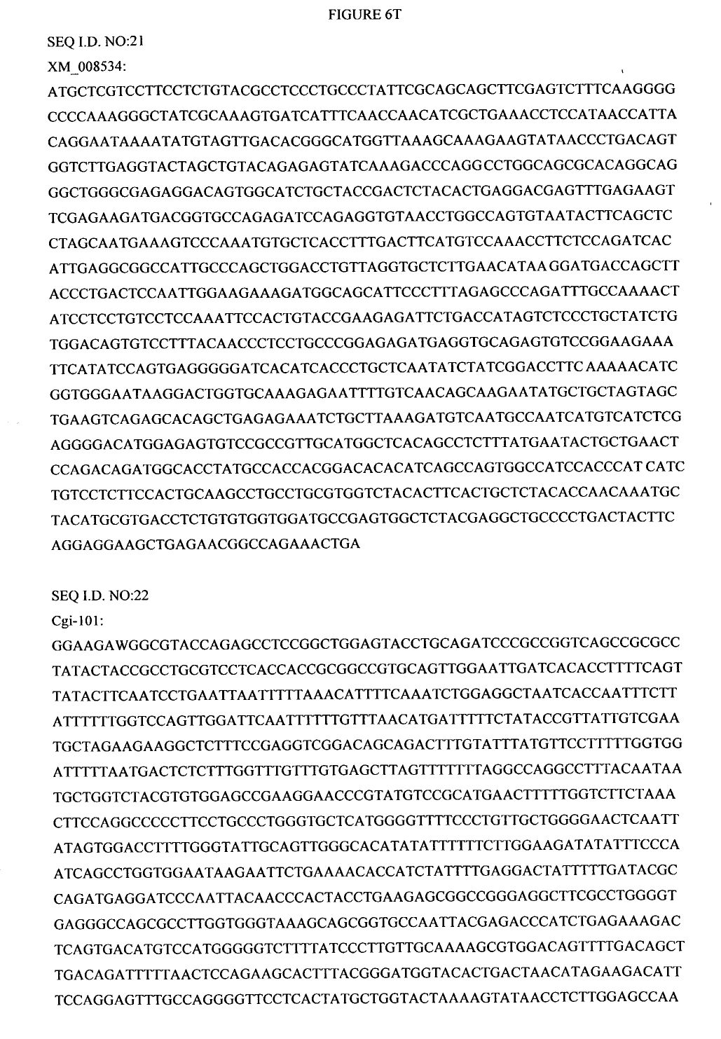

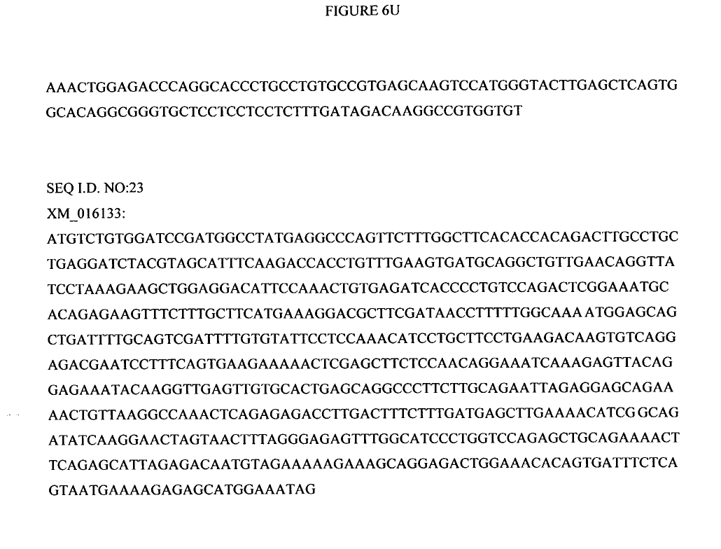

Preferred nucleic acid sequences that encode for a modified susceptible KiflC amino acid sequence may suitably comprise any of the SEQ ID NOS 1 through 23 as shown in FIGS. 6A-6U, as well as sequences that have a substantial sequence identity to SEQ ID NOS 1 through 23, e.g. at least about 70, 75, 80, 85, 90 or 95 percent sequence identity to any one or more of those sequences. Also preferred nucleic acid sequences that encode for a modified susceptible KifC1 amino acid sequence comprise a sequence that will hybridize under normal or high stringency conditions (as such conditions are defined immediately below) to any of the SEQ ID NOS 1 through 23 as shown in FIGS. 6A-6U.

Preferred vectors include viral vectors, fusion proteins and chemical conjugates. Retroviral vectors include moloney murine leukemia viruses. DNA viral vectors are preferred. Viral vectors can be chosen to introduce the genes to cells of choice. Such vectors include pox vectors such as orthopox or avipox vectors, herpesvirus vectors such as herpes simplex I virus (HSV) vector (Geller et al., 1995, J. Neurochem. 64: 487; Lim et al., 1995, in DNA Cloning: Mammalian Systems, D. Glover, ed., Oxford Univ. Press, Oxford, England; Geller et al., 1990, Proc. Natl. Acad. Sci. U.S.A. 87: 1149), adenovirus vectors (LeGal LaSalle et al., 1993, Science 259: 988; Davidson et al., 1993, Nat. Genet. 3: 219; Yang et al., 1995, J. Virol. 69: 2004) and adeno-associated virus vectors (Kaplitt et al., 1994, Nat. Genet. 8: 148).

Pox viral vectors introduce the gene into the cells cytoplasm. Avipox virus vectors result in only short term expression of the nucleic acid. Adenovirus vectors, adeno-associated virus vectors and herpes simplex virus vectors are preferred for introducing the nucleic acid into neural cells. The adenovirus vector results in a shorter term expression (about 2 months) than adeno-associated virus (about 4 months), which in turn is shorter than HSV vectors. The vectors can be introduced by standard techniques, e.g. infection, transfection, transduction or transformation. Examples of modes of gene transfer include for example, naked DNA calcium phosphate precipitation, DEAE dextran, electroporation, protoplast fusion, lipofection, cell microinjection and viral vectors.

The vector can be employed to target essentially any desired target cell. For example, stereotaxic injection can be used to direct the vectors (e.g. adenovirus, HSV) to a desired location. Other methods that can be used include catheters, intravenous, parenteral, intraperitoneal, and subcutaneous injection, and oral or other known routes of administration.

Another preferred method is DNA immunization. DNA immunization employs the subcutaneous injection of a plasmid DNA (pDNA) vector encoding a specific anthrax resistance protein. The pDNA sequence is taken up by antigen presenting cells (APC). Once inside the cell, the DNA encoding protein is transcribed and translated. Higher amounts of the protein present in the antigen presenting cell such as the macrophage may overcome the defect that renders cells susceptible to the lethal effects of the toxin. The results of such a phenotypic rescue are shown in the examples which follow.

Genetic constructs comprise a nucleotide sequence that encodes the KiflC nucleic acid sequence of choice and preferably includes an intracellular trafficking sequence operably linked to regulatory elements needed for gene expression.

When taken up by a cell, the genetic construct(s) may remain present in the cell as a functioning extrachromosomal molecule and/or integrate into the cell's chromosomal DNA. DNA may be introduced into cells where it remains as separate genetic material in the form of a plasmid or plasmids. Alternatively, linear DNA which can integrate into the chromosome may be introduced into the cell. When introducing DNA into the cell, reagents which promote DNA integration into chromosomes may be added. DNA sequences which are useful to promote integration may also be included in the DNA molecule. Alternatively, RNA may be administered to the cell. It is also contemplated to provide the genetic construct as a linear minichromosome including a centromere, telomeres and an origin of replication. Gene constructs may remain part of the genetic material in attenuated live microorganisms or recombinant microbial vectors which live in cells. Gene constructs may be part of genomes of recombinant viral vaccines where the genetic material either integrates into the chromosome of the cell or remains extrachromosomal.

Genetic constructs include regulatory elements necessary for gene expression of a nucleic acid molecule. The elements include: a promoter, an initiation codon, a stop codon, and a polyadenylation signal. In addition, enhancers may be required for gene expression of the sequence of choice, for example, the protective KiflC gene, variants or fragments thereof, that encodes the resistance to the toxic effects of LeTx. It is necessary that these elements be operably linked to the sequence that encodes the desired proteins and that the regulatory elements are operable in the individual to whom they are administered.

Initiation codons and stop codons are generally considered to be part of a nucleotide sequence that encodes the immunogenic target protein. However, it is necessary that these elements are functional in the individual to whom the gene construct is administered. The initiation and termination codons must be in frame with the coding sequence.

Promoters and polyadenylation signals used must be functional within the cells of the individual.

Examples of promoters useful to practice the present invention, especially in the production of a genetic vaccine for humans, include but are not limited to promoters from Simian Virus 40 (SV40), Mouse Mammary Tumor Virus (MMTV) promoter, Human Immunodeficiency Virus (HIV) such as the HIV Long Terminal Repeat (LTR) promoter, Moloney virus, ALV, Cytomegalovirus (CMV) such as the CMV immediate early promoter, Epstein Barr Virus (EBV), Rous Sarcoma Virus (RSV) as well as promoters from human genes such as human Actin, human Myosin, human Hemoglobin, human muscle creatine and human metallothionein.

Examples of polyadenylation signals useful to practice the present invention, especially in the production of a genetic vaccine for humans, include but are not limited to SV40 polyadenylation signals and LTR polyadenylation signals. In particular, the SV40 polyadenylation signal which is in pCEP4 plasmid (Invitrogen, San Diego Calif.), referred to as the SV40 polyadenylation signal, is used.

In addition to the regulatory elements required for DNA expression, other elements may also be included in the DNA molecule. Such additional elements include enhancers. The enhancer may be selected from the group including but not limited to: human Actin, human Myosin, human Hemoglobin, human muscle creatine and viral enhancers such as those from CMV, RSV and EBV.

Genetic constructs can be provided with mammalian origin of replication in order to maintain the construct extrachromosomally and produce multiple copies of the construct in the cell. For example, plasmids pCEP4 and pREP4 from Invitrogen (San Diego, Calif.) contain the Epstein Barr virus origin of replication and nuclear antigen EBNA-1 coding region which produces high copy episomal replication without integration.

In order to maximize protein production, regulatory sequences may be selected which are well suited for gene expression in the cells the construct is administered into. Moreover, codons may be selected which are most efficiently transcribed in the cell. One having ordinary skill in the art can produce DNA constructs which are functional in the cells.

The method of the present invention comprises the steps of administering nucleic acid molecules to tissue of the individual. In some preferred embodiments, the nucleic acid molecules are administered intramuscularly, intranasally, intraperatoneally, subcutaneously, intradermally, or topically or by lavage to mucosal tissue selected from the group consisting of vaginal, rectal, urethral, buccal and sublingual.

In some embodiments, the nucleic acid molecule is delivered to the cells in conjunction with administration of a facilitating agent. Facilitating agents are also referred to as polynucleotide function enhancers or genetic vaccine facilitator agents. Facilitating agents are described in e.g. International Application No. PCT/US94/00899 filed Jan. 26, 1994 and International Application No. PCT/US95/04071 filed Mar. 30, 1995, both incorporated herein by reference. Facilitating agents which are administered in conjunction with nucleic acid molecules may be administered as a mixture with the nucleic acid molecule or administered separately simultaneously, before or after administration of nucleic acid molecules.

In some preferred embodiments, the genetic constructs of the invention are formulated with or administered in conjunction with a facilitator selected from the group consisting of, for example, benzoic acid esters, anilides, amidines, urethans and the hydrochloride salts thereof such as those of the family of local anesthetics. The facilitating agent is administered prior to, simultaneously with or subsequent to the genetic construct. The facilitating agent and the genetic construct may be formulated in the same composition.

In some embodiments of the invention, the individual is first subject to injection of the facilitator prior to administration of the genetic construct. That is, for example, up to a about a week to ten days prior to administration of the genetic construct, the individual is first injected with the facilitator. In some embodiments, the individual is injected with the facilitator about 1 to 5 days; in some embodiments 24 hours, before or after administration of the genetic construct. Alternatively, if used at all, the facilitator is administered simultaneously, minutes before or after administration of the genetic construct. Accordingly, the facilitator and the genetic construct may be combined to form a single pharmaceutical composition.

In some embodiments, the genetic constructs are administered free of facilitating agents, that is in formulations free from facilitating agents using administration protocols in which the genetic constructions are not administered in conjunction with the administration of facilitating agents.

Nucleic acid molecules which are delivered to cells according to the invention may serve as genetic templates for proteins that function as prophylactic and/or therapeutic immunizing agents. In preferred embodiments, the nucleic acid molecules comprise the necessary regulatory sequences for transcription and translation of the coding region in the cells of the animal.

To further define nucleic acid sequences important for conferring resistance, the invention provides for mutants of KiflC in the proposed resistant-conferring sites and other regions in KiflC. Additionally, the KiflC protein-encoding nucleic acid sequences of choice can be mutated in vitro or in vivo, to create and/or destroy translation, initiation, and/or termination sequences, or to create variations in coding regions and/or form new restriction endonuclease sites or destroy preexisting ones, to facilitate further in vitro modification. Any technique for mutagenesis known in the art can be used, including but not limited to, in vitro site-directed mutagenesis (Hutchinson et al., 1978, J. Biol. Chem. 253: 6551; Zoller and Smith, 1984, DNA 3:479-488; Oliphant et al., 1986, Gene 44: 177; Hutchinson et al., 1986, Proc. Natl. Acad. Sci. U.S.A. 83: 710; and others). PCR techniques are preferred for site directed mutagenesis (see Higuchi, 1989, “Using PCR to Engineer DNA”, in PCR Technology: Principles and Applications for DNA Amplification, H. Erlich, ed., Stockton Press, Chapter 6, pp. 61-70).

Various methods known to those skilled in the art can be used to express and produce the gene conferring resistance to LeTx. For example, the identified and isolated gene can be inserted into an appropriate cloning vector. A large number of vector-host systems known in the art may be used. Possible vectors include, but are not limited to, plasmids or modified viruses, but the vector system must be compatible with the host cell used. Examples of vectors include, but are not limited to, E. coli, bacteriophages such as lambda derivatives, or plasmids such as pBR322 derivatives or pUC plasmid derivatives, e.g., pGEX vectors, pmal-c, pFLAG, etc. The insertion into a cloning vector can, for example, be accomplished by ligating the DNA fragment into a cloning vector that has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences. Recombinant molecules can be introduced into host cells via transformation, transfection, infection, electroporation, etc., so that many copies of the gene sequence are generated. Preferably, the cloned gene is contained on a shuttle vector plasmid, which provides for expansion in a cloning cell, e.g., E. coli, and facile purification for subsequent insertion into an appropriate expression cell line, if such is desired. For example, a shuttle vector, which is a vector that can replicate in more than one type of organism, can be prepared for replication in both E. coli and Saccharomyces cerevisiae by linking sequences from an E. coli plasmid with sequences from the yeast 2 plasmid.

In an alternative method, the desired gene may be identified and isolated after insertion into a suitable cloning vector in a “shot gun” approach. Enrichment for the desired gene, for example, by size fractionation, removal of highly-repetitive sequences, subtractive or otherwise selective hybridization, and other methods as may be known in the art, can be done before insertion into the cloning vector.

The nucleotide sequence coding for KiflC protein, or functional fragments, derivatives or analogs thereof, including a chimeric protein, thereof, can be inserted into an appropriate expression vector, i.e., a vector which contains the necessary elements for the transcription and translation of the inserted protein-coding sequence. Such elements are termed herein a “promoter.” Thus, the nucleic acid encoding a KiflC protein of the invention or functional fragment, derivatives or analogs thereof, is operationally associated with a promoter in an expression vector of the invention. Both cDNA and genomic sequences can be cloned and expressed under control of such regulatory sequences. An expression vector also preferably includes a replication origin. The necessary transcriptional and translational signals can be provided on a recombinant expression vector.

Potential host-vector systems include but are not limited to mammalian cell systems infected with virus (e.g., vaccinia virus, adenovirus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

A recombinant KiflC protein of the invention, may be expressed chromosomally, after integration of the coding sequence by recombination. In this regard, any of a number of amplification systems may be used to achieve high levels of stable gene expression (See Sambrook et al., 1989, supra).

The cell into which the recombinant vector comprising the nucleic acid encoding KiflC protein is cultured in an appropriate cell culture medium under conditions that provide for expression of KiflC protein by the cell.

Any of the methods previously described for the insertion of DNA fragments into a cloning vector may be used to construct expression vectors containing a gene consisting of appropriate transcriptional/translational control signals and the protein coding sequences. These methods may include in vitro recombinant DNA and synthetic techniques and in vivo recombination (genetic recombination).

Expression of KiflC protein may be controlled by any promoter/enhancer element known in the art, but these regulatory elements must be functional in the host selected for expression.

Expression vectors containing a nucleic acid encoding a KiflC protein of the invention can be detected or identified by four general approaches: (a) PCR amplification of the desired plasmid DNA or specific mRNA, (b) nucleic acid hybridization, (c) presence or absence of selection marker gene functions, and (d) expression of inserted sequences. In the first approach, the nucleic acids can be amplified by PCR to provide for detection of the amplified product. In the second approach, the presence of a foreign gene inserted in an expression vector can be detected by nucleic acid hybridization using probes comprising sequences that are homologous to an inserted marker gene. In the third approach, the recombinant vector/host system can be identified and selected based upon the presence or absence of certain “selection marker” gene functions (e.g.,—galactosidase activity, thymidine kinase activity, resistance to antibiotics, transformation phenotype, occlusion body formation in baculovirus, etc.) caused by the insertion of foreign genes in the vector. In another example, if the nucleic acid encoding KiflC protein is inserted within the “selection marker” gene sequence of the vector, recombinants containing the KiflC protein insert can be identified by the absence of the KiflC protein gene function.

A wide variety of host/expression vector combinations may be employed in expressing the DNA sequences of this invention. Useful expression vectors, for example, may consist of segments of chromosomal, nonchromosomal and synthetic DNA sequences. Suitable vectors include derivatives of SV40 and known bacterial plasmids, e.g., E. coli plasmids col E1, pCR1, pBR322, pMal-C2, pET, pGEX (Smith et al., 1988, Gene 67: 31-40), pMB9 and their derivatives, plasmids such as RP4; phage DNAS, e.g., the numerous derivatives of phage, e.g., NM989, and other phage DNA, e.g., M13 and filamentous single stranded phage DNA; yeast plasmids such as the 2 μ plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

For example, in a baculovirus expression systems, both non-fusion transfer vectors, such as but not limited to pVL941 (BamH1 cloning site; Summers), pVL1393 (BamH1, SmaI, XbaI, EcoR1, NotI, XmaIII, BglII, and PstI cloning site; Invitrogen), pVL1392 (BglII, PstI, NotI, XmaIII, EcoRI, XbaI, SmaI, and BamH1 cloning site; Summers and Invitrogen), and pBlueBacIII (BamH1, BglII, PstI, NcoI, and HindIII cloning site, with blue/white recombinant screening possible; Invitrogen), and fusion transfer vectors, such as but not limited to pAc700 (BamH1 and KpnI cloning site, in which the BamH1 recognition site begins with the initiation codon; Summers), pAc701 and pAc702 (same as pAc700, with different reading frames), pAc360 (BamH1 cloning site 36 base pairs downstream of a polyhedron initiation codon; Invitrogen(195)), and pBlueBacHisA, B, C (three different reading frames, with BamH1, BglII, PstI, NcoI, and HindIII cloning site, an N-terminal peptide for ProBond purification, and blue/white recombinant screening of plaques; Invitrogen (220)) can be used.

Mammalian expression vectors contemplated for use in the invention include vectors with inducible promoters, such as the dihydrofolate reductase (DHFR) promoter, e.g., any expression vector with a DHFR expression vector, or a DHFR/methotrexate co-amplification vector, such as pED (PstI, SalI, SbaI, SmaI, and EcoRI cloning site, with the vector expressing both the cloned gene and DHFR; see Kaufman, Current Protocols in Molecular Biology, 16.12 (1991). Alternatively, a glutamine synthetase/methionine sulfoximine co-amplification vector, such as pEE14 (HindIII, XbaI, SmaI, SbaI, EcoRI, and BclI cloning site, in which the vector expresses glutamine synthase and the cloned gene; Celltech). In another embodiment, a vector that directs episomal expression under control of Epstein Barr Virus (EBV) can be used, such as pREP4 (BamH1, SfiI, XhoI, NotI, NheI, HindIII, NheI, PvuII, and KpnI cloning site, constitutive RSV-LTR promoter, hygromycin selectable marker; Invitrogen), pCEP4 (BamH1, SfiI, XhoI, NotI, NheI, HindIII, NheI, PvuII, and KpnI cloning site, constitutive hCMV immediate early gene, hygromycin selectable marker; Invitrogen), pMEP4 (KpnI, PvuI, NheI, HindIII, NotI, XhoI, SfiI, BamH1 cloning site, inducible methallothionein IIa gene promoter, hygromycin selectable marker: Invitrogen), pREP8 (BamH1, XhoI, NotI, HindIII, NheI, and KpnI cloning site, RSV-LTR promoter, histidinol selectable marker; Invitrogen), pREP9 (KpnI, NheI, HindIII, NotI, XhoI, SfiI, and BamHI cloning site, RSV-LTR promoter, G418 selectable marker; Invitrogen), and pEBVHis (RSV-LTR promoter, hygromycin selectable marker, N-terminal peptide purifiable via ProBond resin and cleaved by enterokinase; Invitrogen). Selectable mammalian expression vectors for use in the invention include pRc/CMV (HindIII, BstXI, NotI, SbaI, and ApaI cloning site, G418 selection; Invitrogen), pRc/RSV (HindIII, SpeI, BstXI, NotI, XbaI cloning site, G418 selection; Invitrogen), and others. Vaccinia virus mammalian expression vectors (see, Kaufman, 1991, supra) for use according to the invention include but are not limited to pSC11 (SmaI cloning site, TK- and .beta.-gal selection), pMJ601 (SalI, SmaI, AflI, NarI, BspMII, BamHI, ApaI, NheI, SacII, KpnI, and HindIII cloning site; TK- and -gal selection), and pTKgptF1S (EcoRI, PstI, SalI, AccI, HindII, SbaI, BamHI, and Hpa cloning site, TK or XPRT selection).

Yeast expression systems can also be used according to the invention to express KifC1 polypeptides. For example, the non-fusion pYES2 vector (XbaI, SphI, ShoI, NotI, GstXI, EcoRI, BstXI, BamH1, SacI, Kpn1, and HindIII cloning sit; Invitrogen) or the fusion pYESHisA, B, C (XbaI, SphI, ShoI, NotI, BstXI, EcoRI, BamH1, SacI, KpnI, and HindIII cloning site, N-terminal peptide purified with ProBond resin and cleaved with enterokinase; Invitrogen), to mention just two, can be employed according to the present invention.

Once a particular recombinant DNA molecule is identified and isolated, several methods known in the art may be used to propagate it. Once a suitable host system and growth conditions are established, recombinant expression vectors can be propagated and prepared in quantity. As previously explained, the expression vectors which can be used include, but are not limited to, the following vectors or their derivatives: human or animal viruses such as vaccinia virus or adenovirus; insect viruses such as baculovirus; yeast vectors; bacteriophage vectors (e.g., lambda), and plasmid and cosmid DNA vectors, to name but a few.

A preferred vector for the present invention is a moloney murine leukemia virus derived vector.

Vectors are introduced into the desired host cells by methods known in the art, e.g., transfection, electroporation, microinjection, transduction, cell fusion, DEAE dextran, calcium phosphate precipitation, lipofection (lysosome fusion), use of a gene gun, or a DNA vector transporter (see, e.g., Wu et al., 1992, J. Biol. Chem. 267: 963-967; Wu and Wu, 1988, J. Biol. Chem. 263: 14621-14624; Hartmut et al., Canadian Patent Application No. 2,012,311, filed Mar. 15, 1990).

A preferred method of use for the invention is to treat mammals against adverse (lethal) effects of LeTx. As described above various nucleic acid and amino acid sequences can be used to achieve this. A therapeutic composition as used herein, can include e.g. any of the above viruses or vectors containing the entire nucleic acid sequence of the KiflC molecule, variants and fragments thereof; modified nucleic acid sequences of the KiflC molecule; the entire amino acid sequence of the KiflC molecule or fragments thereof; modified amino acid fragments of the KiflC molecule or any peptides embodied in the invention.

The therapeutic composition may be introduced in a suitable carrier. For example, sterile saline solution or sterile phosphate buffered saline.

Another preferred method is using the above-described vectors, or other vectors well known in the art, for introducing vectors into cells or tissues which are equally suitable for use in vivo, in vitro, and ex vivo. For ex vivo therapy, vectors may be introduced into stem cells taken from the patient and clonally propagated for autologous transplant back into that same patient. Macrophages also can be employed. Delivery by transfection, by liposome injections, or by polycationic amino polymers may be achieved using methods which are well known in the art (see, e.g., Goldman et al., 1997, Nature Biotechnology 15: 462-466).

Any of the therapeutic methods described above may be applied to any subject in need of such therapy, including, for example, mammals such as livestock such as sheep, goats, cattle and horses; pets such as dogs, cats and rabbits; preferably, primates such as monkeys; and, most preferably, humans.

### Example 6

Kits According to the Invention

Diagnostic and research reagent kits are also provided which include components to determine identity of the KiflC allele in a patient or other test subject. Thus, the kit may contain a sample of the KiflC gene, an allele or fragment thereof, or expression product of the KiflC gene, an allele or fragment thereof. The kit also may contain instructions (written) for conducting the diagnostic assay. The kit also may contain an assay or test support, typically a solid support, and other materials such as positive control samples, negative control samples, cells, enzymes, detection labels, buffers, etc.

### Example 7

Epitope-Tagged Versions of KiflC

We have cloned one resistant KiflC allele (from C57BL/6J), 2 susceptible variants (from C3H/HeJ and CAST/Ei), and one “motor-dead” allele (with an artificially introduced mutation that disables the motor) into mammalian expression vectors (pcDNA3.1) that fuse myc- and his-tags to the C-terminus of the protein. these constructs were transfected into 3T3 cells, and express proteins of the correct molecular weight on a Western probed with anti-myc antibodies.

Intracellular localization of epitope-tagged KiflC: transient transfection of the myc-tagged constructs into NIH3T3 cells and immunocytofluorescence analysis of the cells using anti-myc antibody showed similar staining patterns with each of the B6, C3H, and CAST forms of the protein (a representative example is shown below and more accurately in Figure A of the Appendix). This staining pattern, has a few notable features. First, while we do not see evidence of golgi stacks in our micrographs, but a higher concentration of staining in a single discrete perinuclear region in every cell is seen. Second, distributed punctate staining is observed throughout the cytosol. Both of these features most closely resemble the staining pattern seen by Nakajima et al, instead of the results seen by Dorner et al. Third, most cells show very intense staining at the tips of extended portions of the cell membrane (pseudopods).

Further Uses

The invention is particularly useful in the detection or diagnosis of anthrax sensitivity or resistance in an individual. Preferably, such individual is a mammal, including primates, especially a human. The invention additionally is useful in the identification of compositions useful in the prevention or treatment of anthrax infection or its effects. The invention also may be used in the prevention or treatment of anthrax infection. Still further, the invention provides kits and reagents useful in the detection, diagnostic, preventive, therapeutic and drug-screening methods described herein.

The foregoing description of the invention is merely illustrative thereof, and it is understood that variations and modifications thereof can be made without departing from the spirit or scope of the invention as set forth in the following claims.

What is claimed is:

1. An isolated nucleic acid sequence or fragment thereof coding for a modified KiflC amino acid sequence.

2. The nucleic acid sequence of claim 1 wherein the modified susceptible sequence reduces the lethal effects of LeTx on cells by at least about by at least 40 percent as determined by a standard viability assay.

3. The nucleic acid sequence of claim 1 wherein the sequence comprises any one of SEQ ID. NOS. 1 through 23 or fragments thereof.

4 The nucleic acid sequences of claim 3, wherein the fragments comprise about 5 to about 25 bases.

5. The nucleic acid sequence of claim 1 where the sequence comprises a sequence having at least about 80 percent sequence identity to any one of SEQ ID. NOS. 1 through 23.

6. The nucleic acid sequence of claim 1 where the sequence comprises a sequence having at least about 90 percent sequence identity to any one of SEQ ID. NOS. 1 through 23.

7. A nucleic acid sequence that hybridizes under normal stringency conditions to a nucleic acid sequence or functional fragment thereof coding for a modified susceptible KifC1 amino acid sequence.

8. A nucleic acid sequence that hybridizes under high stringency conditions to a nucleic acid sequence or functional fragment thereof coding for a modified susceptible KifC1 amino acid sequence.

9. A nucleic acid sequence of a kinesin family member which sequence confers cellular resistance to the lethal effects of LeTx, as determined by a standard viability test.

10. A recombinant vector comprising a sequence that encodes a LeTx-resistant KiflC protein.

11. A cell comprising a vector of claim 10.

12. The cell of claim 11, wherein such cell is a hematopoietic stem cell.

13. A method for protecting a cell against Bacillus anthracis infection, comprising administering to the cell an effective amount of a nucleic acid sequence coding for a resistant KiflC amino acid sequence or fragment thereof.

14. The method of claim 13, wherein the cell is a human cell.

15. The method of claim 13, wherein the cell is rendered resistant to adverse effects of LeTx.

16. The method of claim 11, wherein the resistant sequence reduces the lethal effects of LeTx on cells by at least about by at least 40 percent as determined by a standard viability assay.

20. The method of any one of claims 11 through 16, wherein the sequence comprises any one of SEQ ID. NOS. 1 through 23.

21. The method of any one of claims 11 through 16, wherein the sequence comprises any mammalian variants of SEQ ID. NOS. 1 through 23.

22. A method for treating a mammal against anthrax, comprising:
administering to the mammal or cells thereof an effective amount a nucleic acid sequence coding for a modified susceptible KiflC amino acid sequence or functional fragment thereof.

23. The method of claim 22 wherein the mammal is a human.

24. The method of claim 22 wherein cells are rendered resistant to adverse effects of LeTx.

25. The method of claims 22 wherein the modified susceptible sequence reduces adverse effects of LeTx on cells by at least about by at least 40 percent as determined by a standard viability assay.

26. The method of claim 22 wherein the sequence comprises any one of SEQ ID. NOS. 1 through 23.

27. The method of claim 22, wherein the sequence comprises human variants.

28. An isolated nucleic acid sequence encoding at least one or more modified amino acid sequences of wild type KiflC; one or more modified allelic amino acid sequences of KiflC, the modification comprising at least one amino acid substitution or deletion in an epitope capable of conferring resistance to adverse effects of LeTx.

29. The nucleic acid sequence of claim 38 wherein the nucleic acid sequence comprises one or more modified gene fragments of protective KiflC.

30. A modified susceptible KiflC amino acid sequence or functional fragment thereof.

31. The sequence of claim 30 wherein the sequence comprises at least about 30 amino acid residues.

32. The sequence of claim 30 or wherein the modified susceptible sequence reduces the lethal effects of LeTx on cells by at least about by at least 40 percent as determined by a standard viability assay.

33. A pharmaceutical composition comprising a nucleic acid sequence or amino acid sequence of any one of claims 1-9 or 30-32 and a pharmaceutically acceptable carrier.

34. A method for identifying a compound that interacts with KiflC gene, variants or fragments thereof, or oligopeptides, comprising
contacting a candidate agent with the KiflC gene, an allele or fragment thereof, or expression product thereof, and
performing a detection step to detect interaction between said KiflC gene, an allele or fragment thereof, or expression product thereof.

35. The method of claim 34, wherein the candidate compound is selected from the group consisting of a protein, a peptide, an oligopeptide, a nucleic acid, a small organic molecule, a polysaccharide and a polynucleotide.

36. The method of claim 34 or 35 wherein said KiflC gene, variants or fragments thereof, or oligopeptides or candidate compound comprises a label.

37. A method for identifying compounds that interact with KiflC gene, variants, or fragments thereof, or expression products thereof, comprising:
providing a KiflC gene, allele or fragment thereof, or oligopeptide expression product thereof, and,
contacting a candidate compound with the KiflC gene, allele or fragment or oligopeptide; and
detecting interaction of the candidate compound with the KiflC gene, allele or fragment or oligopeptide.

38. The method of claim 37 wherein the KiflC gene, variant or fragment oligopeptide are provided on a solid support.

39. The method of claim 37 or 38 wherein binding of the candidate compound with the KiflC gene, variant or fragment or oligopeptide is detected.

40. The method of any one of claims 37 through 39 wherein the candidate compound is selected from the group consisting of a protein, a peptide, an oligopeptide, a nucleic acid, a small organic molecule, a polysaccharide and a polynucleotide.

41. The method of claim any one of claims 37 through 40 wherein the KiflC gene, variant or fragment or peptides or candidate compound comprises a detectable label.

42. A drug compound obtained by a method of any one of claims 34 through 41.

43. A kit comprising a KiflC gene, variant or fragment thereof, or expression product thereof.

44. The kit of claim 43 comprising written instructions for a diagnostic assay using the KiflC gene, allele or fragment thereof, or expression product thereof.

45. A method for identifying a component of a test sample, comprising:
contacting a test sample with the KiflC gene, variant or fragment thereof, or expression product of the KiflC gene, variant or fragment thereof; and detecting interaction of the test sample with the KiflC gene, an variant or fragment thereof, or expression product of the KiflC gene, variant or fragment thereof.

46. The method of claim 45 wherein the test sample is a mammalian tissue or fluid sample.

47. A method for identifying one or more genes that mediate anthrax susceptibility in a mammal comprising:
hybridizing an isolated nucleic acid sequence with a KiflC nucleic acid probe to form a hybridized molecule; and
detecting sequences hybridized to the probe.

48. The method of claim 47, wherein the anthrax susceptibility gene, allele or fragment oligopeptide are provided on a solid support.

49. The method of claim 47 or 48 wherein binding of the candidate gene and/or gene product with the anthrax susceptibility gene, allele or fragment or oligopeptide is detected.

50. The method of any one of claims 47 through 49, wherein the anthrax susceptibility gene is KiflC

51. A method for characterizing variants of genes that mediate anthrax susceptibility as being linked to the susceptible or resistant phenotype comprising:
administering to a mammalian cell i) a candidate gene and/or gene product and ii) brefeldin A; and,
culturing the cell with anthrax toxin.

52. The method of claim 51 wherein a mammalian cell is intoxicated in the presence of anthrax toxin if the mammalian cell comprises a susceptibility gene or variants thereof; or a mammalian cell does not become intoxicated in the presence of anthrax toxin if the mammalian cell comprises a resistant gene or variants thereof.

53. The method of claim 51 or 52 wherein the resistant or susceptible gene or variants thereof are KiflC.

54. A method for screening a mammal for anthrax susceptibility or resistance, comprising:
contacting a candidate gene and/or gene product with an anthrax susceptibility or anthrax resistance gene, an allele or fragment thereof; and
analyzing the mixture of the candidate gene and/or gene product and the anthrax susceptibility or resistance gene, an allele or fragment thereof.

55. The method of claim 54, wherein the anthrax susceptibility or resistance gene, allele or fragment oligopeptide are provided on a solid support.

56. The method of claim 54 or 55 wherein binding of the candidate gene and/or gene product with the anthrax susceptibility or resistance gene, allele or fragment or oligopeptide is detected.

57. The method of claim 51, wherein the anthrax susceptibility or resistance genotype is further correlated to susceptibility or resistance phenotype by steps comprising:
administering to a mammalian cell i) a candidate gene and/or gene product and ii) brefeldin A; and,
culturing the cell with anthrax toxin.

58. The method of claim 57, wherein a mammalian cell is intoxicated in the presence of anthrax toxin if the mammalian cell comprises a susceptibility gene or variants thereof; or a mammalian cell does not become intoxicated in the presence of anthrax toxin if the mammalian cell comprises a resistant gene or variants thereof.

59. A method for identifying anthrax therapeutic compounds that interact with anthrax susceptibility genes, variants or fragments thereof, or expression products thereof, comprising:
administering to a mammalian cell a candidate compound and brefeldin A; and,
culturing the cell with anthrax toxin; and,
determining the intoxication of the cultured cell.

60. The method of claim 59, wherein the cell is intoxicated in the presence of anthrax toxin if the candidate compound does not inhibit the intoxicating effects of the anthrax toxin; or the cell does not become intoxicated in the presence of anthrax toxin if the candidate compound inhibits the intoxicating effects of the anthrax toxin.

61. The method of claim 60, wherein the anthrax susceptibility gene, allele or fragment oligopeptide are provided on a solid support.

62. The method of any one of claims 59 through 61, wherein binding of the candidate compound with the anthrax susceptibility gene, allele or fragment or oligopeptide is detected.

63. The method of any one of claims 59 through 62 wherein the candidate compound is selected from the group consisting of a protein, a peptide, an oligopeptide, a nucleic acid, a small organic molecule, a polysaccharide and a polynucleotide.

64. The method of any one of claims 59 through 63 wherein the anthrax susceptibility gene, allele or fragment or peptides or candidate compound comprises a detectable label.

65. The method of any one of claims 59 through 64 wherein the anthrax susceptibility gene, allele or fragment comprises at least a portion of a sequence of KiflC.

66. The method of any one of claims 59 through 65 wherein interaction of a candidate compound to KiflC gene product is detected by immunofluorescence and/or immunoprecipitation.


SEQUENCE LISTING (23 sequences)

SEQ ID NO: 1 - DNA, 591 bp, Unknown Organism
  Description of Unknown Organism KiflC nucleic acid sequence
atgtgtactggaaagtgtgcgcgctgcctggggctctccctcatccctctctccctggtctgcatcgtggccaacgccctcctgctggtacctgatgggaagaccacctggacggacggcaacctcagcttgcaagtttggctcatgggtggcttcattggagggggcctgatggtgctgtgtccaggaattgcagcggtccgggcagggggaaagggctgctgcggtgcaggttgctgtggcaaccgctgcaggatgctgcgctccgtcttctcctccgcctttggggtgcttggcgccatctactgcctgtcagtggcgggagctgggctccgaattggacccaaatgcttaatagacaacaagtgggactaccacttccaagaaacagaaggcgcttacttgcgaaatgacactctttggaatttgtgtgaggcgccacctcacgtggtaccctggaatgtgacactcttctcaatcctggtggtcgcatcaagtctggaacttgtgctgtgtggaattcagctggtgaatgcgacctttggtgtgttgtgtggcgattgcaggaaaaaggagggtgcagctcactga

SEQ ID NO: 2 - DNA, 609 bp, Unknown Organism
  Description of Unknown Organism KiflC nucleic acid sequence
atggctgtgcagtttaatgggggcgtggttctaggagcggactccaggacaaccactgggtcctacatcgccaatcgagtgactgacaagctgacccctatccacgatcacatcttctgctgccgctcaggctcagccgctgatacccaagcagtggcagacgctgtcacttaccagcttggtttccacagtattgaactgaacgagcctccactagtccacacagccgccagtctctttaaggagatgtgttaccggtacagagaagatctgatggcaggaatcatcattgcaggctgggaccctcaagaaggagggcaggtgtactctgttcccatggggggtatgatggtaagacagtcctttgccatcggaggctccgggagctcgtacatctatggctatgttgatgctacgtatcgggaaggcatgaccaaggacgaatgtctgcagttcactgccaatgctctcgctttggccatggaacgcgacggctccagtggaggggtgatccgcttggcagccattcaggagtcaggggtagagcggcaggtgcttttgggagaccaaatccccaagttcaccattgccacgttgccacctccctga

SEQ ID NO: 3 - DNA, 2802 bp, Unknown Organism
  Description of Unknown Organism KiflC nucleic acid sequence
atgactgtaacccagaagaacctctttccctatggggactatctgaactccagccagttgcacatggagccagatgaggttgacactctgagggaaggagaggatccagctgatcgaatgcatccctatctggccatctatgaccttcagcctctgaaagcacaccccttggtgttcgcccctggggtccctgttatagcccaggtggtgggcaccgaaagatacaccagcggatccaaggtgggaacctgtactctatattctgttcgcttgacgcatggtgactttacctggacaaccaagaagaagttccgacactttcaggagctgcatcgggacctccagagacacaaagtcttgatgagtctgctccctttggctcgctttgctgtgacccattctccagcccgagaggcagccgccgaggatataccctccctaccccgaggaggttctgagggctctgccagacacacagccagcaaacagaaatacttggaaaattacctcaaccgcctcctgaccatgtctttctatcgcaattaccacgccatgacagaatttctggaagtcagtcaactttcctttatcccagaccttggctccaaaggactggaaggggtgatccggaagcgctcgggcgggcatcgagttcccggcttcaccttctgtggccgagaccaagtttgttatcgatggtccaagaggtggctggtggtgaaggactccttcctgctgtacatgcgcccggagaccggcgccatctcatttgttcagctttttgaccctggctttgaggtccaggtcggaaaaaggagcacagagacgcggtatggggtgaggatcgacacctcccacaggtccctgattctcaaatgcagcagctaccggcaggcacggtggtggggccaggagatcacggagctggcacagggttcgggcagagattttctacagctacatcagcatgacagctatgccccaccccggcccggcaccctggcccggtggtttgtgaatggggcaggttactttgctgctgtggcagatgccatcctgcgagctcaagaggagattttcatcacagactggtggttgagtcctgaaatttacctgaagcgtccagcccattccgacgactggagactggacattatgctcaagaggaaggcggaagaaggtgtccgagtttccatactgctgtttaaggaagtggagctggccttgggcatcaacagtggctacagcaagaggacgctgatgctgctgcatcccaacataaaggtgatgcgacacccagaccttgtgacactgtgggctcatcacgagaagctcctggtggtagaccaagtggtggcattcttgggcgggctggacctggccttcggccgctgggatgacgtgcaataccgactgactgacctgggtgacccctctgaacctgtacatttacagactcccacactaggttcagaccctgcagccactccagacctctcgcataaccaattcttctggctgggaaaggactacagcaacctcatcaccaaggactgggtgcagctggaccggccttttgaagatttcatcgacagggagaccacacccaggatgccatggagggatgttggagtggttgtacacggagtagctgccagggaccttgcccggcacttcatccagcgctggaatttcaccaagaccaccaaggccaggtataagacacctttgtacccctacctgctgcccaagtccaccagcactgcaaacaatctccccttcatgatcccaggcgggcagtgtgccactgtgcaggtcttgaggtctgtggatcgatggtcagcagggacattggagaactccatcctcaatgcctacctacataccattcgagagagccagcactttctctacattgagaatcagttcttcattagctgctcagatgggcgaacagttctgaacaaggtgggcgatgagattgtggacagaatcctgaaggctcacgaacaggggcagtgtttccgagtctacttgcttctgcctttgctccctggctttgagggggacatctccacagggggtggtaactccatccaggctattctgcacttcacctacaggaccctgtgtcgtggggaacattcaatcctacatcgtctcaaagcagccatggggactgcgtggcgagattacatgtccatctgtgggcttcgcacccatggagagctgggcgggcacccaatctctgagctcatctatatccacagcaagatgctcattgcggatgacagaacagtcatcattggttctgcgaacatcaatgacaggagcttgctggggaagcgtgacagtgagctagccatcctgatcaaggacacagaaatggaaccatccctcatggatggggtggagtaccaggcaggcagatttgccttgagtttgcggaagcactgtttcagtgtgattcttggggcaaatacctggccagacctggatctccgagaccctgtctgtgatgacttcttccagctgtggcaagaaacagcggagaacaatgccaccatctatgagcagatcttccgctgcctgccgtccaatgctacccgttccctgcgggctctccgggagtatgtggctgtggagtccttggctacagtcagcccttctttggctcagtctgagcttgcccacatccagggccacctagttcacttccccctcaagtttctggaggacgagtccttgttgcccccactggggagtaaagaagggatgatacctttagaagtgtggacatag

SEQ ID NO: 4 - DNA, 3903 bp, Unknown Organism
  Description of Unknown Organism KiflC nucleic acid sequence
atgggcgacccagcccccgcccgcagcctggacgacatcgacctgtctgccctgcgggaccctgcaggaatctttgagctggtggaggtggttggcaatggaacctatggacaggtatacaaggggcggcacgtcaagactgggcagctggctgccattaaggtcatggatgtcacagaggatgaggaggaagagatcaaacaggaaatcaacatgttaaagaagtactctcaccatcgcaatattgccacctactatggggcctttatcaagaagagccctcctgggaacgatgaccagctctggctggtgatggagttctgcggtgctggttcagtgaccgacctggtaaagaacacaaaagggaacgcactgaaggaggattgcattgcttacatctgcagggagattctcaggggtcttgcccatctccatgcccacaaggtgatccacagagatatcaagggacaaaatgtgctgctgacagagaatgctgaagtcaagctagtggattttggggtgagtgctcagctggaccgcactgtgggcaggcggaacactttcattggaaccccatactggatggctccagaagtcattgcctgtgacgagaaccccgatgccacctatgactacaggagtgacatttggtctctaggaatcacagccattgaaatggcagagggagccccccctctgtgtgacatgcaccctatgcgggccctcttcctcatccctcggaaccctccccccaggctcaagtcaaagaaatggtctaagaagttcactgacttcatcgacacgtgtctcatcaagacttacctgagccgcccacccaccgaacagttactcaaattccccttcatccgagaccagcccacggagcggcaggtccgcatccagctcaaggaccacatcgaccgctcgcggaagaagcggggtgagaaagaggagacagagtatgagtacagcggcagtgaggaggaagacgacagccatggagaggaaggcgagccaagctccatcatgaatgtgcccggtgagtccacactgcgcagagaattcctcagactccagcaggagaataagagcaactctgaggctttaaagcagcagcagcagctgcagcaacagcagcagcgggacccggaggcacacatcaaacacctgctgcaccagcggcagcgtcgcatagaggagcagaaggaggagcggcgacgtgtggaggagcaacagcggcgagagcgagaacagcgtaagctacaagagaaggagcagcagcggcgattggaagacatgcaagccctacgacgagaggaagagaggcggcaagcagagcgggaacaggaatacaagcggaagcagctggaggagcagcggcagtcagagcggctgcagagacagctgcagcaggagcacgcctacctcaagtccctgcagcagcagcagcagcagcagcagctccagaagcagcagcagcagcagcagcagatcctgcctggagacaggaagcccctgtatcattacggtcggggcattaatcctgctgacaagccagcatgggcccgcgaggtggaagagagagcacggatgaacaagcagcagaactctcccttggcgaaggcgaagccaagcagtgcggggccagagccccccatctcccaggcctctcctagccccccaggacctctttcccagactcctcctatgcagaggcctgtggagccccaggaaggaccgcacaagagcctggtggcacaccgggtcccactgaagccatatgcagcacctgtaccccgatcccagtccctgcaggaccagccgactcgaaacctggctgccttcccagcctcccacgaccctgaccctgctgctgtccctacacccactgccacacccagtgcccgaggagctgtcatccgccagaattcagaccccacctctgaagggccagggcctagcccaaaccctccatcctgggttcggcctgataatgaggctccacctaaggttccacagaggacctcttctatcgccactgcccttaacaccagtggggccggagggtcccggccagctcaggctgtccgtgccagtaaccctgacctcaggaggagtgaccctggctgggagcgctcagacagtgtcctcccggcctcccacggccacctccctcaggctggctccttggagcggaaccgaaaccgtgtgggagcctccacaaaactggatagctctccagtgctctcccctgggaacaaagccaagcctgaagaccaccgctcaaggccaggccggcccgcagactttgtgttgctcaaagagcggactctggatgaggcccctaagcctcccaagaaggccatggactactcctcatccagtgaggaggtggaaagcagtgaagaggaggaggaggaaggcgatggggagccgtcagaggggagcagagacactcccgggggccgcagtgatggtgatacagacagcgtcagcaccatggtggttcatgatgttgaggagatatccgggacccagccctcatatggcggcggcaccatggtggtccagcgtactcctgaagaggaacgaagcctgctgcttgctgatagcaatggctacacaaacctgcctgatgtggtccagcccagccactcacctactgagaacagcaaaggtcaaagccctccaacaaaggatggaggcagtgattaccagtctcgtgggctggtaaaggccccaggaaagagctcattcaccatgtttgtggatctagggatctaccagcctggaggcagtggggacaccatccctatcacagccctagtgggtggagaaggtggtcgccttgatcaactgcagttcgatgtgaggaagggctctgtggtcaacgtcaatcccaccaacacccgagctcatagtgaaactcctgaaattcgcaagtacaagaagcgattcaactcagagatcctatgtgcagctctctggggggtcaacctcctagtgggcacagagaatgggctgatgttgctggaccgaagtgggcagggcaaggtgtatggacttattgggcgacgacgcttccagcaaatggatgtcttagaagggctcaacttgctcatcaccatctcagggaaaaggaacaaactgcgggtatattacctgtcctggcttcggaacaagatcctacacaatgacccagaggtggaaaagaagcaggggtggaccaccgtgggggacatggagggctgcggccactaccgtgttgtgaaatatgaacggattaagttcctggtcattgccctgaagaactccgtggaggtttatgcctgggctcccaaaccctaccacaaattcatggccttcaagtcctttgctgacctccctcaccgccctctactggtggacctgacagtagaggagggacagcggctcaaggtcatctatggctccagtgctggcttccatgctgtggatgttgattctgggaacagctatgacatctacatccctgtacatatccagagccagatcacaccccacgccatcatcttcctccccaacactgatggcatggagatgctgctgtgctatgaagatgagggtgtctatgtcaacacttacgggcggatcatcaaggatgtggtgctgcagtggggagagatgcccacctctgtggcctacatctgctccaaccagataatgggctggggtgagaaggccatagagatccgctctgtggagacaggccacctagatggggtcttcatgcacaaacgagcccagaggctcaagttcctgtgtgagcgcaatgacaaggtgttttttgcctctgtccgctctggaggaagcagccaagtttactttatgactctgaaccgtaactgcatcatgaactggtga

SEQ ID NO: 5 - DNA, 1091 bp, Unknown Organism
  Description of Unknown Organism KiflC nucleic acid sequence
atggcggcgacggcgagtcccggggctggccggatggacgggaaaccccgtacctcccctaagtctgtcaagttcctgtttgggggcctggctgggatgggtgctacagtctttgtgcagcccctggacctggtgaagaaccggatgcagttgagtggtgaaggggccaagactcgagagtacaaaaccagtttccatgccctcaccagcatcctgaagacagaaggcctgaagggcatttacactgggctgtcagctggtctactgcgccaggccacctacaccactactcgccttggaatatatactgtgttgtttgagcgcctgactggggctgatggtacaccccctggctttcttctgaaagccctgattggcatgactgcaggtgcaactggtgcatttgtgggaacgccagctgaggtggctctcatcaggatgactgctgatggtcggcttccagctgaccagcgccgtggctacaaaaatgtgtttaatgccctagttaggattgccagggaagaaggagtccccacactgtggcggggctgcatccctaccatggctcgagctgtcgttgtcaatgccgcccagcttgcctcttactctcaatctaagcagttcttgctggactcaggctacttctctgacaatattctctgccacttctgcgccagcatgatcagtggcctcgttaccactgctgcttccatgcctgtggacatcgtcaaaactaggatccagaatatgcggatgattgatgggaagccagaatacaagaatgggctggatgtgctgctgaaagtcgtccgctatgagggtttcttcagcctgtggaagggcttcacaccatactatgcccgactgggcccccacactgtcctcaccttcatcttcttggaacagatgaacaaggcctacaagcgtctcttcctcagtggctgatctggcatggtcccaggactagtgtgccaggcttccaagccatccactgctattacagtcgctatgccctggaggacctggattctgctaccctgggctattctatttattttccctctccagtgtggttttctcctttgcggtaa

SEQ ID NO: 6 - DNA, 1044 bp, Unknown Organism
  Description of Unknown Organism KiflC nucleic acid sequence
atgcaccctgcggccttcccgcttcctgttgtcgtggctactgtactgtggggagcagcccctgtccgagggctgattcgagcgacttcggagcacaatgccagcatggactttgcagaccttccagctctgtttggggccactctgagcgacgagggactgcaggggttccttgtggaggcccacccagaaaatgcctgcggtcctattgccccaccaccctcagccccggtcaatgggtcagtctttattgcactgcttcgaagattcgactgcaactttgacctcaaggtcctaaatgctcagaaggctgggtatggtgcagctgtggtgcacaatgtgaattccaatgaactgctgaacatggtgtggaacagcgaggaaatccaacagcagatctggatcccatctgtatttattggagagagaagcgcagagtacctacgagccctttttgtctacgagaagggggctcgggtgcttctggttccagacaacagcttccccttgggctattacctcattcctttcactgggattgtaggattactggttttggccatgggaacagtattgatagttcgttgcatccagcaccggaaacggcttcagcggaaccgacttaccaaagagcaactgaaacagattcctacccatgactatcagaaaggagatgaatatgacgtctgtgccatctgtctggatgaatatgaggatggggacaagcttcgagtacttccctgtgctcatgcttatcacagtcgctgtgtggacccctggctcactcagacccgcaagacctgccccatctgcaaacagcctgtacatcggggtcctggcgatgaggagcaggaagaagaaactcaagagcaagaggaaggcgatgaaggggagccaagggaccaacctgcttcagaatggaccccactcctgggctctagccctactcttcccacctcctttggatccctagcccctgctcccctggtcttccctgggccctccacagatccctcacctccttcatctgctgccctggcctga

SEQ ID NO: 7 - DNA, 423 bp, Unknown Organism
  Description of Unknown Organism KiflC nucleic acid sequence
atggccgggtggaacgcctacatcgacagccttatggcggacgggacctgtcaggacgcggccatcgtaggctacaaggactcgccctccgtctgggccgccgtccccgggaagaccttcgttagcattacgccagctgaggttggtgtcctggtaggcaaagaccggtcaagttttttcgtcaatgggctgacacttgggggccagaaatgttctgtgatccgggactcactgctgcaagacggggaatttacaatggatcttcgtaccaagagcaccggaggagcccccaccttcaatgtcactgtcaccatgactgccaagacgctagtcctgctgatgggcaaagaaggtgtccacggtggtttgatcaacaagaaatgttatgaaatggcctctcacctgcggcgttcccagtactga

SEQ ID NO: 8 - DNA, 1305 bp, Unknown Organism
  Description of Unknown Organism KiflC nucleic acid sequence
atggccatgcaaaaaatcttcgcccgggaaatcctggactccaggggcaaccccacggtggaggtggacctgcacacagccaagggtcgattccgagcagctgtgcccagtggagcttccacgggtatctatgaagcactggaactccgagatggagacaaagcacgatacctggggaaaggagtgctgaaggctgtggaacacatcaacaagactctaggtcctgctctgctggaaaagaaactaagtgttgtggatcaagaaaaagttgacaagttcatgattgagctggacgggaccgagaataagtccaagtttggggccaacgccatcctgggtgtgtccctggctgtctgcaaggctggagcagctgagaaaggggtccctctctaccgacacatcgcagatcttgcaggcaatcccgacctcgtactccctgtgcctgcctttaatgtgatcaacggcggctctcatgctggaaacaagctggccatgcaggagttcatgattctgccagtgggagccagctctttcaaggaagccatgcgcatcggcgctgaggtctaccaccacctcaagggggtcatcaaggccaagtatgggaaggacgccaccaacgtgggggatgagggtggctttgcacccaacatcctggagaacaatgaggccctggagctgctaaagacagccatccaggcagccggttacccggacaaggtggtgatcggcatggatgtagctgcgtctgaattctaccgcaacggcaagtatgatctggacttcaagtcacccgatgaccctgccaggcacatcagtggggagaagcttggggagctgtacaagaacttcatccagaactatcccgtggtctccattgaggacccctttgaccaggatgactgggccacatggacctcattcctctctggggtggacatccagattgtgggagatgacctcacggtaaccaaccccaagaggattgctcaggctgtggagaagaaggcctgcaattgcctgctcctgaaggtcaaccagatcggctccgtgacggagtccatccaggcctgtaaacttgcacaatctaatggctggggagtgatggtgagccaccgctctggggagaccgaagacactttcatcgctgaccttgtggtgggactctgcacaggacagatcaagactggtgctccctgccgttcagagcgtctggcaaaatacaaccagcttatgaggattgaggaggctcttggggacaaagctgtctttgctggaagaaagttccgtaatccaaaggccaaatga

SEQ ID NO: 9 - DNA, 651 bp, Unknown Organism
  Description of Unknown Organism KiflC nucleic acid sequence
atggagaaaccacccagcctcggtgaccaggagagtcggcgcaaggcccgagagcaggctgcccgtctgaagaaactacaagagcaagacaaacagcagaaagtggagtttcggaaaaggatggagaaagaggtgtctgatttcatccaggacagtggacaggtcaagaaaaagtttcagcctatgaacaagatagagcggagcatactacatgatgtggtagaggtggctggcctcacatccttctcctttggagaagatgatgactgtcgctatgtcatgatcttcaaaaaggagtttgcaccctcagatgaagagctagactcctaccgtcatggagaggagtgggacccccagaaggctgaggagaagcggaagctaaaggagctggctcagaagcaggaggaagaggcagcgcagcagggacctgcggttgtgagtcctgccagcgactacaaggacaagtatagccatcttattggcaaaggagcagccaaagatgcagcccacatgctacaggccaacaagacctatggctgtgttcctgtggccaacaagcgggacacacggtccatagaggaggccatgaatgagatcagagccaagaaacgcctgcggcagagtggtgaagagttgccaactacctcctag

SEQ ID NO: 10 - DNA, 3591 bp, Unknown Organism
  Description of Unknown Organism KiflC nucleic acid sequence
atgaataccaaggacaccactgaggttgctgagaatagccaccacctgaagatcttcctccccaagaaattgctggagtgtcttcctcgatgcccactgttgcctccagagcggctccgatggaatacaaatgaggagattgcatcctacctgatcacctttgagaaacacgatgagtggctgtcttgtgccccaaagacaaggcctcagaatggctccattatcctctacaatcgtaagaaggtgaaataccggaaggatggttacctttggaagaaacggaaggatgggaagactacccgagaggaccacatgaaactcaaggtccagggcatggagtgtctctatggctgctacgttcactcttccatcgtccccacattccaccggcgctgctattggctgctccagaaccccgacatcgtccttgtgcactacctgaacgtcccagccctggaggattgtggaaagggctgtagccccatcttttgttccatcagtagcgaccgtcgagaatggctgaagtggtcacgtgaggagctcttgggacagctgaagcccatgtttcatggcatcaagtggagctgtgggaatggggcagaggaattctccgtggaacagttggtgcaacagatcttggacacccacccaaccaagccagcacccagaactcatgcttgtctctgcagtgggggccttggttccgggagccttacccacaaatgcagtagcacgaaacaccgcatcatctctcccaaagtggagccccgagctttagccctggcttctatatcccactccaagccccccgaacccccaccactgatagctccacttcctccagagctccccaaggcacacacctccccatcttcctcctcttcttcctcctcctcctcaggatttgcagaacccctagaaatcaggcctagccctcctacctctcgagggggttcatcaagaggaggcactgctatcctcctcctaacaggactagagcagcgcgctgggggcctgacacccaccaggcacttggctccgcaggctgagcctagacctcctgtgagcttggccgtggttgtaggttctgagccttctgccccaccggctcctcccagccctgcctttgaccctgatcgttttctcaacagcccccaaaggggccagacatacggagggggccaaggagtaaacccagacttccccgaggcagagggcactcacactccctgtcctgccctggaacctgcagctgccctggagccccaggcggctgctcgaggtctcgctccacagttgggagcaaacgggagaagaggaaacaaattctttatccaagatgatgatagtggggaggaactcaagggtccgggaacggtgccgccggtaccttcatcccctccttcatccccgtcctccccaaccgccttgccgccatcaggcagggcaacgagaggagaagccttgttcggaggctctgctggcagcagcagcgagctcgagcccttcagtctttcgtcattcccagacctcatgggagaactcatcagtgatgaagctccaggtgtccctgcccccgccccccagctctctcctgcgctaaatgccatcacagacttctccccagagtggtcctacccagagggcggggtcaaggtgctcatcacaggcccttggacagaggccgcagagcattactcctgtgtcttcgatcacatcgcagtgccagcctccctggtccagcctggtgtcttacgctgctactgtcctgcccatgaggtagggctggtgtctttgcaggtggcggggcgagagggccccctctctgcctctgtgctctttgagtatcgagcccgccggttcctgtctctgcctagtacacagctcgactggttgtcactggatgacagccagttccggatgtccatcctggagcggctggagcagatggagaagcggatggcagagattgcggcagctgggcaggctcctggtcagggcccagaggctcctccaattcaggatgaaggccagggccctggcttcgaggcacgggtggtggtcttggtagagagcatgatcccgcggtccacttggaggggtcctgaacggctgatccatggaagtcccttccggggcatgagcctcctacatctggctgctgcacagggctacgctcggctcatcgagactctgagccagtggaggagtgtggaaacaggaagcttggacttagagcaagaggttgacccgctcaatgtggaccatttctcttgcacccctctgatgtgggcctgtgctctgggacacttagaagctgctgtgcttcttttctgttggaaccgacaagcactgagcattccggactctctgggccgactccccctgtctgtggctcattctcgtggtcacgtgcgccttgcccgctgccttgaggaactgcagagacaggagctttcagttgagcacccactcgctctatccccaccatcctctagcccagacactggcctgagcagcgcctcgtcaccctccgagctgtcagatggcactttctctgtgacatcagcctactcaagtgccccagatggcagtcctccccctgctcctccgctggcctctgacatttctatggagatgatcccaggccagctttcttgtggtgcccctgagacacccttactcctcatggactatgaagccactaactccaaagaacctgctccctccccgtgcggccccccactagcccaggacaatggggctgctccagaggatgctgacagcccaccagctgtggatgtgatcccggtggacatgatctcactggccaagcagatcatcgacgccacaccagagcggattaaacgagaggacttctcagagttccctgacgctggagcctcaccacgggagcacacaggcaccgtggggctcagtgagaccatgtcctggctggccagttacctggagaatgtggatcatttccccagctcagctcctcccagtgagctgccctttgaacgaggtcgcctcgctatcccccctgcaccttcctgggcagagttcctctctgcatctaccagtggcaagatggaaagtgattttgccctgctgactctctcagatcacgagcagcgggagctgtatgaggcagcacgagtcatccagacagctttccgaaagtacaagggtcggaggctgaaggagcagcaggaggtagccgcagcggtgatccaacgttgttatcggaagtacaagcagtttgcactctataagaaaatgactcaggcggccatcctgattcagagcaagttccgaagctactatgaacagaagcggtttcagcagagccgccgagcagcggtgcttatccagcagcactaccgctcctaccgccgcaggccggggcctcctcaccggccctcaggcccccttcctgctcgaaacaagggcacctttctcaccaagaagcaagaccaggcagcccggaaaataatgaggttcctgcggcgctgccgacacagaatgagggaactgaagcagaaccaggagctggaggggctgccccagccaggactggccacctga

SEQ ID NO: 11 - DNA, 3517 bp, Unknown Organism
  Description of Unknown Organism KiflC nucleic acid sequence
atctggggccaggacaccagctgaggagggcaggagatcctggaagctatggctggagcctccgtgaaagttgcagtgagggttcggccctttaatgcccgtgagaccagccaggatgccaagtgtgtggtcagcatgcagggcaacaccacctccatcattaatcccaaacagagcaaggatgcccccaaaagcttcaccttcgactattcttactggtcgcatacttcggtggaggatccgcagtttgcatctcagcagcaggtgtaccgagacataggagaagagatgctactccatgcctttgaaggctacaatgtgtgcatctttgcctacgggcagacgggggctgggaagtcctacaccatgatggggcggcaggagccggggcagcagggcattgtgcctcagctctgtgaggacctcttctctcgagtcaatgtgaaccagagtgcccagctttcctattcagtagaggtgagctatatggaaatctattgcgagcgagtacgagacctcttgaaccccaagagtcggggctctctgcgggtccgggagcaccccatcctaggcccgtacgtgcaagacctgtctaaactggctgtgacttcctatgcagacattgccgacctcatggactgtggaaataaggcaagaaccgtggctgccaccaacatgaacgagaccagcagccgctcccatgccgtctttactattgtctttacccagcgatcccatgaccagcttactggcctggattcagaaaaggtcagtaagatcagcttggtggacctcgccgggagtgagcgggctgactcctcaggggcccgggggatgcgtctaaaggaaggcgccaacatcaataagtccctgactacgcttgggaaggtgatctcagccctggcagatttgcaatcaaagaagcggaagtcggatttcatcccctatagagactcagtgcttacctggctgctcaaggagaatttgggtgggaactctcgcacagcaatgattgcagccctgagtcctgctgacatcaattacgaggagacactcagcactctcaggtatgcagaccgcaccaagcagatccgatgcaatgctgtcatcaacgaggaccccaatgcccggcttatccgggagctgcaggaggaggtggcccggctgcgggacctgctgatggctcaggggctgtcagcctctgctctaggaggtctaaaggtggaagaggggagtcctggaggtgttctgccccctgcatcatctccccccgccccagcttcaccctcatctcctcccccacataatggggagctggagccctcattctcacccagtgccgagccccagattgggcctgaggaggccatggagaggctgcaggaaacagagaagatcatagctgagctgaatgagacttgggaggagaagcttcggaagacagaggctctgagaatggagagagaagcattgctggctgagatgggcgtggctgtccgggaggatggcggtactgtgggtgtcttctctccaaaaaagactccccacctggtaaatctgaatgaggaccccttgatgtctgaatgtcttctctaccacatcaaagatggcgtcaccagggttggccaagtagatgtggacatcaagttgactggccagttcatccgggagcagcattgcctcttccgcagcatccctcagcctgatggagaagtgatggttactctggagccttgtgaaggagctgagacatatgtcaatgggaagctcgtgacggagccgctggtgctgaagtcaggaaacaggattgtaatgggcaagaaccatgttttccgcttcaatcacccggagcaggcgcggctggaacgagaacgtggggtccccccgcccccaggacctccctcggagcctgtggactggaactttgctcagaaggagctgctggagcagcaaggcatagacatcaagctggagatggagaagaggctgcaggacttggagaatcagtaccggaaagagaaggaagaggctgaccttctgctggagcagcagcggctgtatgcagactctgacagcggggaagactctgacaagcgttcttgtgaagaaagctggcggctgatttcctccttgcgggagcagctgccccctaccacagtacagaacattgtcaagcgctgtggcctgcctagtagtggcaagcgcagggcccctcgaagagtctatcagatccctcagcgacggcggctccagggcaaagacccccgatgggccaccatggctgacctgaagatgcaggcagtaaaggagatatgctatgaggtggccctagccgacttccgccacggacgagcagaaattgaagccctggctgctctcaagatgagggagctgtgccgaacctacggcaagccagagggtcctggagatgcctggagagccgtggccagggatgtctgggatactgtgggtgaagaagaaggatgtggaggtggcggaggtggcagtgaggagggagcccgtggggcagaggtggaggacctccgggctcacatcgacaagctgacagggatcctgcaggaggtgaagctccagaacagcagcaaggaccgcgagctgcaggccctgcgggaccgcatgctccgcatggagagggtcatcccgctgactcaggatcttgaggatgataatgacgagtctggtttggtcacctgggccccaccggaagggccagaagcagtagaggagacagtgcccaacgaccactcaccagctgtccggcccacctcaccacctctgtccagctgggagcgggtgtcgagattgatggaagaggaccctgccttccgtcgcggccgccttcgctggctcaagcaggagcagctgcggctgcagggactgcagggcgctgggggccggggtgggggactgcgcaggcctccagcccgctttgtgcccccgcatgattgcaagctgcgcttccccttcaaaagcaaccctcagcacagggagtcctggccaggaatggggagtggagaggccccagctcctcagccccctgaagaggtcactgtccccccagcaccccctaaccgtaggcctccaagtccccgaagaccccaccgttctcgcaggaattccctagatggagggagccgatctcggggagggggttctacacagccagaaccccagcacttacggccccaaaagcacaacggttatccccaacagccccagcccagcccagcccagcggccagggccccgctacccaccatacactacccctccgcgaatgcgacggcagcgctcagcccctgacctcaaagagagtggggcagctgtgtgagtcccacatgatgggcagacagggcctagtggggcccttgttaggaggaaggtgcccactgcttccccaagaagccctggggcaaggaggccagaaagcagagagcaagagagagggaaggtccgagtaggtggtagaagatgccagagagtgtgctggaggctga

SEQ ID NO: 12 - DNA, 3517 bp, Unknown Organism
  Description of Unknown Organism KiflC nucleic acid sequence
atctggggccaggacaccagctgaggagggcaggagatcctggaagctatggctggagcctccgtgaaagttgcagtgagggttcggccctttaatgcccgtgagaccagccaggatgccaagtgtgtggtcagcatgcagggcaacaccacctccatcattaatcccaaacagagcaaggatgcccccaaaagcttcaccttcgactattcttactggtcgcatacttcggtggaggatccgcagtttgcatctcagcagcaggtgtaccgagacataggagaagagatgctactccatgcctttgaaggctacaatgtgtgcatctttgcctacgggcagacgggggctgggaagtcctacaccatgatggggcggcaggagccggggcagcagggcattgtgcctcagctctgtgaggacctcttctctcgagtcaatgtgaaccagagtgcccagctttcctattcagtagaggtgagctatatggaaatctattgcgagcgagtacgagacctcttgaaccccaagagtcggggctctctgcgggtccgggagcaccccatcctaggcccgtacgtgcaagacctgtctaaactggctgtgacttcctatgcagacattgccgacctcatggactgtggaaataaggcaagaaccgtggctgccaccaacatgaacgagaccagcagccgctcccatgccgtctttactattgtctttacccagcgatcccatgaccagcttactggcctggattcagaaaaggtcagtaagatcagcttggtggacctcgccgggagtgagcgggctgactcctcaggggcccgggggatgcgtctaaaggaaggcgccaacatcaataagtccctgactacgcttgggaaggtgatctcagccctggcagatttgcaatcaaagaagcggaagtcggattttatcccctatagagactcagtgcttacctggctgctcaaggagaatttgggtgggaactctcgcacagcaatgattgcagccctgagtcctgctgacatcaattacgaggagacactcagcactctcaggtatgcagaccgcaccaagcagatccgatgcaatgctgtcatcaacgaggaccccaatgcccggcttatccgggagctgcaggaggaggtggcccggctgcgggacctgctgatggctcaggggctgtcagcctctgctctaggaggtctaaaggtggaagaggggagtcctggaggtgttctgccccctgcatcatctccccccgccccagcttcaccctcatctcctcccccacataatggggagctggagccctcattctcacccagtgccgagccccagattgggcctgaggaggccatggagaggctgcaggaaacagagaagatcatagctgagctgaatgagacttgggaggagaagcttcggaagacagaggctctgagaatggagagagaagcattgctggctgagatgggcgtggctgtccgggaggatggcggtactgtgggtgtcttctctccaaaaaagactccccacctggtaaatctgaatgaggaccccttgatgtctgaatgtcttctctaccacatcaaagatggcgtcaccagggttggccaagtagatgtggacatcaagttgactggccagttcatccgggagcagcattgcctcttccgcagcatccctcagcctgatggagaagtgatggttactctggagccttgtgaaggagctgagacatatgtcaatgggaagctcgtgacggagctgctggtgctgaagtcaggaaacaggattgtaatgggcaagaaccatgttttccgcttcaatcacccggagcaggcgcggctggaacgagaacgtggggtccccccgcccccaggacctccctcggagcctgtggactggaactttgctcagaaggagctgctggagcagcaaggcatagacatcaagctggagatggagaagaggctgcaggacttggagaatcagtaccggaaagagaaggaagaggctgaccttctgctggagcagcagcggctgtatgcagactctgacagcggggaagactctgacaagcgttcttgtgaagaaagctggcggctgatttcctccttgcgggagcagctgccccctaccacagtacagaacattgtcaagcgctgtggcctgcctagtagtggcaagcgcagggcccctcgaagagtctatcagatccctcagcgacggcggctccagggcaaagacccccgatgggccaccatggctgacctgaagatgcaggcagtaaaggagatatgctatgaggtggccctagccgacttccgccacggacgagcagaaattgaagccctggctgctctcaagatgagggagctgtgccgaacctacggcaagccagagggtcctggagatgcctggagagccgtggccagggatgtctgggatactgtgggtgaagaagaaggatgtggaggtggcggaggtggcagtgaggagggagcccgtggggcagaggtggaggacctccgggctcacatcgacaagctgacagggatcctgcaggaggtgaagctccagaacagcagcaaggaccgcgagctgcaggccctgcgggaccgcatgctccgcatggagagggtcatcccgctgactcaggatcttgaggatgataatgacgagtctggtttggtcacctgggccccaccggaagggccagaagcagtagaggagacagtgcccaacgaccactcaccagctgtccggcccacctcaccacctctgtccagctgggagcgggtgtcgagattgatggaagaggaccctgccttccgtcgcggccgccttcgctggctcaagcaggagcagctgcggctgcagggactgcagggcgctgggggccggggtgggggactgcgcaggcctccagcccgctttgtgcccccgcatgattgcaagctgcgcttccccttcaaaagcaaccctcagcacagggagtcctggccaggaatggggagtggagaggccccagctcctcagccccctgaagaggtcactgtccccccagcaccccctaaccgtaggcctccaagtccccgaagaccccaccgttctcgcaggaattccctagatggagggagccgatctcggggagggggttctacacagccagaaccccagcacttacggccccaaaagcacaacggttatccccaacagccccagcccagcccagcccagcggccagggccccgctacccaccatacactacccctccgcgaatgcgacggcagcgctcagcccctgacctcaaagagagtggggcagctgtgtgagtcccacatgatgggcagacagggcctagtggggcccttgttaggaggaaggtgcccactgcttccccaagaagccctggggcagggaggctagaaagcagagagcaagagagagggaaggtccgagtaggtggtagaagatgccagagagtgtgctggaggctga

SEQ ID NO: 13 - DNA, 3518 bp, Unknown Organism
  Description of Unknown Organism KiflC nucleic acid sequence
atctggggccaggacaccagctgaggagggcaggagatcctggaagctatggctggagcctccgtgaaagttgcagtgagggttcggccctttaatgcccgtgagaccagccaggatgccaagtgtgtggtcagcatgcagggcaacaccacctccatcattaatcccaaacagagcaaggatgcccccaaaagcttcaccttcgactattcttactggtcgcatacttcggtggaggatccgcagtttgcatctcagcagcaggtgtaccgagacataggagaagagatgctactccatgcctttgaaggctacaatgtgtgcatctttgcctacgggcagacgggggctgggaagtcctacaccatgatggggcggcaggagccggggcagcagggcattgtgcctcagctctgtgaggacctcttctctcgagtcaatgtgaaccagagtgcccagctttcctattcggtagaggtgagctatatggagatctattgcgagcgagtacgagacctcttgaaccccaagagtcggggctctctgcgggtccgggagcaccccatcctaggcccgtacgtgcaagacctgtctaaactggctgtgacttcctatgcagacattgccgacctcatggactgtggaaataaggcaagaaccgtggctgccaccaacatgaacgagaccagcagccgctcccatgccgtctttactattgtctttacccagcgatcccatgaccagcttactggcctggattcagaaaaggtcagtaagatcagcttggtggacctcgccgggagtgagcgggctgactcctcaggggcccgggggatgcgtctaaaggaaggcgccaacatcaataagtccctgactacgcttgggaaggtgatctcagccctggcagatttgcaatcaaagaagcggaagtcggatttcatcccctatagagactcagtgcttacctggctgctcaaggagaatttgggtgggaactctcgcacagcaatgattgcagccctgagtcctgctgacatcaattacgaggagacactcagcactctcaggtatgcagaccgcaccaagcagatccgatgcaatgctgtcatcaacgaggaccccaatgcccggcttatccgggagctgcaggaggaggtggcccggctgcgggacctgctgatggctcaggggctgtcagcctctgctctaggaggtctaaaggtggaagaggggagtcctggaggtgttctgccccctgcatcatctccccccgccccagcttcaccctcatctcctccaccacataatggggagctggagccctcattctcacccagtgccgagccccagattgggcctgaggaggccatggagaggctgcaggaaacagagaagatcatagctgagctgaatgagacgtgggaggagaagcttcggaagacagaggctctgagaatggagagagaagcattgctggctgagatgggcgtggctgtccgggaggatggcggtactgtgggtgtcttctctccaaaaaagactccccacctggtaaatctgaatgaggaccccttgatgtctgaatgtcttctctaccacatcaaagatggcgtcaccagggttggccaagtagatgtggacatcaagttgactggccagttcatccgggagcagcattgcctcttccgcagcatccctcagcctgatggagaagtgatggttactctggagccttgtgaaggagctgagacatatgtcaatgggaagctcgtgacggagccgctggtgctgaagtcaggaaacaggattgtaatgggcaagaaccatgttttccgcttcaatcacccggagcaggcgcggctggaacgagaacgtggggtccccccgcccccaggacctccctcggagcctgtggactggaactttgctcagaaggagctgctggagcagcaaggcatagacatcaagctggagatggagaagaggctgcaggacttggagaatcagtaccggaaagagaaggaagaggctgaccttctgctggaacagcagcggctgtatgcagactctgacagtggggaagactctgacaagcgttcttgtgaagaaagctggcggctgatttcctccttgcgggagcagctgccccctaccacagtacagaacattgtcaagcgctgtggcctgcctagtagtggcaagcgcagggcccctcgaagagtctatcagatccctcagcgacggcggctccagggcaaagacccccgatgggccaccatggctgacctgaagatgcaggcagtaaaggagatatgctatgaggtggccctagccgacttccgccacggacgagcagaaattgaagccctggctgctctcaagatgagggagctgtgccgaacctacggcaagccagagggtcctggagatgcctggagagccgtggccagggatgtctgggatactgtgggtgaagaagaaggatgtggaggtggcggaggtggcagtgaggagggagcccgtggggcggaggtggaggacctccgggctcacatcgacaagctgacagggatcctgcaggaggtgaagctccagaacagcagcaaggaccgcgagctgcaggccctgcgggaccgcatgctccgcatggagagggtcatcccgctgactcaggatcttgaggatgataatgacgagtctggtttggtcacctgggccccaccggaagggccagaagcagtagaggagacagtgcccaacgaccactcaccagctgtccggcccacctcaccacctctgtccagctgggagcgggtgtcgagattgatggaagaggaccctgccttccgtcgcggccgccttcgctggctcaagcaggagcagctgcggctgcagggactgcagggcgctgggggccggggtgggggactgcgcaggcctccagcccgctttgtgcccccgcatgattgcaagctgcgcttccccttcaaaagcaaccctcagcacagggagtcctggccaggaatggggagtggagaggccccagctcctcagccccctgaagaggtcactgtccccccagcaccccctaaccgtaggcctccaagtccccgaagaccccaccgtcctcgcaggaattccctagatggagggagccgatctcggggagggggttctacacagccagaaccccagcacttacggccccaaaagcacaacggttatccccaacagccccagcccagcccagcccagcggccagggccccgctacccaccatacactacccctccgcgaatgcgacggcagcgctcagcccctgacctcaaagagagtggggcagctgtgtgagtcccacatgatgggcagacagggcctagtggggcccttgttaggaggaaggtgcccactgcttccccaagaagccctggggcagggaggccagaaagcagagagcaagagagagggaaggtccgagtaggtggttagaagatgccagagagtgtgctggaggctga

SEQ ID NO: 14 - DNA, 3517 bp, Unknown Organism
  Description of Unknown Organism KiflC nucleic acid sequence
atctggggccaggacaccagctgaggagggcaggagatcctggaagctatggctggagcctccgtgaaagttgcagtgagggttcggccctttaatgcccgtgagaccagccaggatgccaagtgtgtggtcagcatgcagggcaacaccacctccatcattaatcccaaacagagcaaggatgcccccaaaagcttcaccttcgactattcttactggtcgcatacttcggtggaggatccgcagtttgcatctcagcagcaggtgtaccgagacataggagaagagatgctactccatgcctttgaaggctacaatgtgtgcatctttgcctacgggcagacgggggctgggaagtcctacaccatgatggggcggcaggagccggggcagcagggcattgtgcctcagctctgtgaggacctcttctctcgagtcaatgtgaaccagagtgcccagctttcctattcggtagaggtgagctatatggagatctattgcgagcgagtacgagacctcttgaaccccaagagtcggggctctctgcgggtccgggagcaccccatcctaggcccgtacgtgcaagacctgtctaaactggctgtgacttcctatgcagacattgccgacctcatggactgtggaaataaggcaagaaccgtggctgccaccaacatgaacgagaccagcagccgctcccatgccgtctttactattgtctttacccagcgatcccatgaccagcttactggcctggattcagaaaaggtcagtaagatcagcttggtggacctcgccgggagtgagcgggctgactcctcaggggcccgggggatgcgtctaaaggaaggcgccaacatcaataagtccctgactacgcttgggaaggtgatctcagccctggcagatttgcaatcaaagaagcggaagtcggatttcatcccctatagagactcagtgcttacctggctgctcaaggagaatttgggtgggaactctcgcacagcaatgattgcagccctgagtcctgctgacatcaattacgaggagacactcagcactctcaggtatgcagaccgcaccaagcagatccgatgcaatgctgtcatcaacgaggaccccaatgcccggcttatccgggagctgcaggaggaggtggcccggctgcgggacctgctgatggctcaggggctgtcagcctctgctctaggaggtctaaaggtggaagaggggagtcctggaggtgttctgccccctgcatcatctccccccgccccagcttcaccctcatctcctccaccacataatggggagctggaaccctcattctcacccagtgccgagccccagattgggcctgaggaggccatggagaggctgcaggaaacagagaagatcatagctgagctgaatgagacgtgggaggagaagcttcggaagacagaggctctgagaatggagagagaagcattgctggctgagatgggcgtggctgtccgggaggatggcggtactgtgggtgtcttctctccaaaaaagactccccacctggtaaatctgaatgaggaccccttgatgtctgaatgtcttctctaccacatcaaagatggcgtcaccagggttggccaagtagatgtggacatcaagttgactggccagttcatccgggagcagcattgcctcttccgcagcatccctcagcctgatggagaagtgatggttactctggagccttgtgaaggagctgagacatatgtcaatgggaagctcgtgacggagccgctggtgctgaagtcaggaaacaggattgtaatgggcaagaaccatgttttccgcttcaatcacccggagcaggcgcggctggaacgagaacgtggggtccccccgcccccaggacctccctcggagcctgtggactggaactttgctcagaaggagctgctggagcagcaaggcatagacatcaagctggagatggagaagaggctgcaggacttggagaatcagtaccggaaagagaaggaagaggctgaccttctgctggagcagcagcggctgtatgcagactctgacagtggggaagactctgacaagcgttcttgtgaagaaagctggcggctgatttcctccttgcgggagcagctgccccctaccacagtacagaacattgtcaagcgctgtggcctgcctagtagtggcaagcgcagggcccctcgaagagtctatcagatccctcagcgacggcggctccagggcaaagacccccgatgggccaccatggctgacctgaagatgcaggcagtaaaggagatatgctatgaggtggccctagccgacttccgccacggacgagcagaaattgaagccctggctgctctcaagatgagggagctgtgccgaacctacggcaagccagagggtcctggagatgcctggagagccgtggccagggatgtctgggatactgtgggtgaagaagaaggatgtggaggtggcggaggtggcagtgaggagggagcccgtggggcggaggtggaggacctccgggctcacatcgacaagctgacagggatcctgcaggaggtgaagctccagaacagcagcaaggaccgcgagctgcaggccctgcgggaccgcatgctccgcatggagagggtcatcccgctgactcaggatcttgaggatgataatgacgagtctggtttggtcacctgggccccaccggaagggccagaagcagtagaggagacagtgcccaacgaccactcaccagctgtccggcccacctcaccacctctgtccagctgggagcgggtgtcgagattgatggaagaggaccctgccttccgtcgcggccgccttcgctggctcaagcaggagcagctgcggctgcagggactgcagggcgctgggggccggggtgggggactgcgcaggcctccagcccgctttgtgcccccgcatgattgcaagctgcgcttccccttcaaaagcaaccctcagcacagggagtcctggccaggaatggggagtggagaggccccagctcctcagccccctgaagaggtcactgtccccccagcaccccctaaccgtaggcctccaagtccccgaagaccccaccgtcctcgcaggaattccctagatggagggagccgatctcggggagggggttctacacagccagaaccccagcacttacggccccaaaagcacaacggttatccccaacagccccagccttacccagcccagcggccagggccccgctacccaccatacactacccctccgcgaatgcgacggcagcgctcagcccctgacctcaaagagagtggggcagctgtgtgagtcccacatgatgggcagacagggcctagtggggcccttgttaggaggaaggtgcccactgcttccccaagaagccctggggcagggaggccagaaagcagagagcaagagagagggaaggtccgagtaggtggtagaagatgccagagagtgtgctggaggctga

SEQ ID NO: 15 - DNA, 3518 bp, Unknown Organism
  Description of Unknown Organism KiflC nucleic acid sequence
atctggggccaggacaccagctgaggagggcaggagatcctggaagctatggctggagcctccgtgaaagttgcagtgagggttcggccctttaatgcccgtgagaccagccaggatgccaagtgtgtggtcagcatgcagggcaacaccacctccatcattaatcccaaacagagcaaggatgcccccaaaagcttcaccttcgactattcttactggtcgcatacttcggtggaggatccgcagtttgcatctcagcagcaggtgtaccgagacataggagaagagatgctactccatgcctttgaaggctacaatgtgtgcatctttgcctacgggcagacgggggctgggaagtcctacaccatgatggggcggcaggagccggggcagcagggcattgtgcctcagctctgtgaggacctcttctctcgagtcaatgtgaaccagagtgcccagctttcctattcggtagaggtgagctatatggagatctattgcgagcgagtacgagacctcttgaaccccaagagtcggggctctctgcgggtccgggagcaccccatcctaggcccgtacgtgcaagacctgtctaaactggctgtgacttcctatgcagacattgccgacctcatggactgtggaaataaggcaagaaccgtggctgccaccaatatgaacgagaccagcagccgctcccatgccgtctttactattgtctttacccagcgatcccatgaccagcttactggcctggattcagaaaaggtcagtaagatcagcttggtggacctcgccgggagtgagcgggctgactcctcaggggcccgggggatgcgtctaaaggaaggcgccaacatcaataagtccctgactacgcttgggaaggtgatctcagccctggcagatttgcaatcaaagaagcggaagtcggatttcatcccctatagagactcagtgcttacctggctgctcaaggagaatttgggtgggaactctcgcacagcaatgattgcagccctgagtcctgctgacatcaattacgaggagacactcagcactctcaggtatgcagaccgcaccaagcagatccgatgcaatgctgtcatcaacgaggaccccaatgcccggcttatccgggagctacaggaggaggtggcccggctgcgggacctgctgatggctcaggggctgtcagcctctgctctaggaggtctaaaggtggaagaggggagtcctggaggtgttctgccccctgcatcatctccccccgccccagcttcaccctcatctcctccgccacataatggggagctggagccctcattctcacccagtgccgagccccagattgggcctgaggaggccatggagaggctgcaggaaacagagaagatcatagctgagctgaatgagacttgggaggagaagcttcggaagacagaggctctgagaatggagagagaagcattgctggctgagatgggcgtggctgtccgggaggatggcggtactgtgggtgtcttctctccaaaaaagactccccacctggtaaatctgaatgaggaccccttgatgtctgaatgtcttctctaccacatcaaagatggcgtcaccagggttggccaagtagatgtggacatcaagttgactggccagttcatccgggagcagcactgtctcttccgcagcatccctcagcctgatggagaagtgatggttactctggagccttgtgaaggagctgagacatatgtcaatgggaagctcgtgacggagccgctggtgctgaagtcaggaaacaggattgtaatgggcaagaaccatgttttccgcttcaatcacccggagcaggcgcggctggaacgagaacgtggggtccccccgcccccaggacctccctcggagcctgtggactggaactttgctcagaaggagctgctggagcagcaaggcatagacatcaagctggagatggagaagaggctgcaggacttggagaatcagtaccggaaagagaaggaagaggctgaccttctgctggagcagcagcggctgtatgcagactctgacagtggggaagactctgacaagcgttcttgtgaagaaagctggcggctgatttcctccttgcgggagcagctgccccctaccacagtacagaacattgtcaagcgctgtggcctgcctagtagtggcaagcgcagggcccctcgaagagtctatcagatccctcagcgacggcggctccagggcaaagacccccgatgggccaccatggctgacctgaagatgcaggcagtaaaggagatatgctatgaggtggccctagccgacttccgccacggacgagcagaaattgaagccctggctgctctcaagatgagggagctgtgccgaacctacggcaagccagagggtcctggagatgcctggagagccgtggccagggatgtctgggatactgtgggtgaagaagaaggatgtggaggtggcggaggtggcagtgaggagggagcccgtggggcagaggtggaggacctccgggctcacatcgacaagctgacagggatcctgcaggaggtgaagctccagaacagcagcaaggaccgcgagctgcaggccctgcgggaccgcatgctccgcatggagagggtcattccgctgactcaggatcttgaggatgataatgaagagtctggtttggtcacctgggccccaccggaagggccagaagcagtagaggagacagtgcccaacaaccactcaccagctgtccggcccacctcaccacctctatccagctgggagcgggtgtcaagattgatggaagaggaccctgccttccgtcgcggccgccttcgctggctcaagcaggagcagctgcggctgcagggactgcagggtgctgggggccggggtgggggactgcgcaggcctccagcccgctttgtgcccccgcatgattgcaagctgcgcttccccttcaaaagcaaccctcagcacagggagtcctggccaggaatggggagtggagaggccccagctcctcagccccctgaagaggtcactgcccccccagctccccctaaccgtaggcctccaagtccccgaagaccccaccgtcctcgcaggaattccctagatggaggaagccgatctcggggagggggttctacacagccagaaccccagcacttacggccccaaaagcacaactgttatccccaacagccccaaccttacccagcccagcggccagggccccgctacccaccatacactacccctccgcgaatgcgacggcagcgctcagcccctgacctcaaagagagtggggcagctgtgtgaatcccacatgatgggcagacagggcctagtggggcccttgttaggaggaaggtgcccactgcttccccaagaagccctggggcaggggaggccagaaagcagagagcaagagagagggaaggtccgagtaggtggtagaagatgccagagagtgtgctggaggctga

SEQ ID NO: 16 - DNA, 935 bp, Unknown Organism
  Description of Unknown Organism KiflC nucleic acid sequence
ggggtgagtgagggtatgtgggcgatcaagcctctccccagttgtgcttgagtaacaccctcagtccacagctgtctccccagctgcttccagtgaacaccccggcagtctaggctcccacagcaatgagttggtggagggacaacttctggatcatcttagctatgtccatcatcttcatctccctggtcctgggtctcatcctgtactgtgtctgcaggtggcagcttagacaaggcaggaactgggaaattgctaagccctcaaaacaggatggaagagatgaagaaaagatgtatgagaatgttcttaattcttcaccaggccagttacctgctctgccacccaggggttcaccttttccaggagacctagccccacaggaagctccaagacaaccctcagcttggtactcatcagtgaagaaagttaggaacaagaaggtctttgctatctcgggctccaccgagccagaaaatgattatgatgatgttgagattccagcaaccaccgaaacccagcactctaaaaccacacctttttggcaagctgaagtgggtttacacagctcgttttagaatactctagaatagccggattataacacaagcacttcctaatccccagaggaagccacctcagccatgtgaaagctacagcagaagacaggacagcttgatgttcccgaggctccagatgtttctgttgctccagatgtttctgctgctccagatgtttctgttgctccaaatatttctgctgctccagatgtttctgttgctccagatgtttctgttgctccagatgctcctgttgctccagatgctcctgatgtttctgacactgcagaagctctaccccaagattctgaggatgtggccttggcacctttgtggaggaagtttccttagtgcagaccactgggcctgtgagag

SEQ ID NO: 17 - DNA, 2589 bp, Unknown Organism
  Description of Unknown Organism KiflC nucleic acid sequence
atggcgcagccgggcccggctccccagcctgacgtttctcttcagcaacgggtagcagaattggaaaaaattaatgcagaatttttacgtgcacagcagcagcttgagcaagaatttaatcaaaagagagcaaaatttaaggagctctatttggctaaagaggaggatctgaagaggcaaaatgcagtattgcaagctgcacaggacgatttgggtcaccttcggacacagctgtgggaggctcaggcagagatggagaacattaaggcaattgccacagtctctgagaatactaagcaagaagctatagatgaagttaaaaggcaatggagagaagaagttgcttcacttcaggctataatgaaagagacagtccgtgactatgagcatcagtttcacctgaggctggagcaggagcgagcacagtgggcacagtatcgagaatctgcagagcgggaaatagctgacttaagaagaaggttgtctgaaggtcaagaggaagaaaatttagaaaatgaaatgaaaaaggcccaagaggatgctgaaaagcttcgttctgtcgtgatgcccatggagaaggaaattgcagctctgaaagataaactgacagaagctgaagacaagattaaagagctggaggcctcaaaggttaaggagctgaatcattatctggaggccgagaagtcttgcaggactgacctggagatgtacgtagctgttttgaacactcagaaatctgtgctacaggaagatgctgagaaattgaggaaagaactgcatgaagtttgccatctcttggaacaagaacgacaacaacacaaccagttaaagcatacatggcagaaggccaacgaccagtttctggaatctcagcgtttgctgatgagagacatgcagagaatggagattgtgttaacttcggaacagctccgacaagttgaagaactaaagaagaaagaccaggaggaggatgaacaacaaagggtcaataagagaaaagataacaagaaaacagataccgaagaagaagtaaaaataccagtagtgtgtgctttaactcaagaagaatcttcaaccccattatcaaatgaagaggagcatttagacagcacccatgggtcagtccattccttggatgcagacttgatgttaccatctggagatccatttagtaaatcggacaatgacatgtttaaagatggactcaggagagctcagtctacagacagcttgggaacctcaagctcattgcaatccaaagctttaggctataactacaaagcgaaatctgctggaaatttggatgagtcggattttggaccactggttggagcagattctgtgtctgagaactttgatactgtgtcccttgggtcactgcagatgccaagtggatttatgttaaccaaagatcaagaaagagcaattaaggcaatgacacctgagcaggaggagacagcatccctcctctccagtgtcacccagggtatggagagtgcatatgtatctcccagtggttaccgcttagtgagtgagacggaatggaatctcctgcagaaagaggtacataatgctggaaataagcttggtagacgttgtgatatgtgttccaattatgaaaaacagttacaaggaattcagattcaggaagctgaaacaagagaccaggtgaaaaaactgcagttaatgctcaggcaagctaatgaccagcttgagaagacgatgaaagagaagcaggagctggaggacttcctcaagcagagtgcggaggactcaagccaccagatatctgcacttgttttaagagcccaagcctctgaggtcttacttgaagagttacaacagagcttttcccaagcaaagagggacgttcaggagcagatggcggtgctgatgcagtcacgtgaacaggtttcagaagagttggtgaggttacagaaagacaacgacagcctccaggggaagcacagcttgcatgtgtcattacagctagcagaagacttcattcttccagacactgtggaggtgctccgggaactggttttaaaatatcgtgagaacattgttcatgtgcgcacagcagcagaccacatggaagagaagctgaaggcggaaatacttttcctaaaagagcaaattcaagcggaacagtgtttaaaagaaaacctggaagagacactacagctggaaatagaaaactgcaaggaggaaatagcttccatttctagtctaaaagctgaattagaaagaataaaggttgaaaagggacagttggaatctacattaagagagaagtcgcaacaacttgagagtctccaagaaatgaaagttaatttagaggaacagttaaagaaagaaactgctgctaaggctactgttgaacagctcatgtttgaagagaagaacaaagctcagaggttgcagacagaattagatgtcagtgaacaagtacagagagattttgtaaaactttctcagactcttcaggtgcagttggagaggatccggcaagcagactccttggagaggatccgggctattctgaatgataccaaactgacagacattaaccagctccctgagacatga

SEQ ID NO: 18 - DNA, 2229 bp, Unknown Organism
  Description of Unknown Organism KiflC nucleic acid sequence
atggcggctgccgtggggcccttgggcgatggggagctgtggcagagctggcttcctaaccacgtcgtgttcttgcggctccgcgagggcgtaagaaaccagagtccagccgaagcggagaagccagcggcttcgacttcaccctcgtgcccgtctctgccgccgcatttgccgacgagaaacctggtcttcggcctcggaggggaactgtttctgtgggacgcagaaggcagcgccttcttggtggttcgccttcgaggccccagcggtggtggcgtggagccccctctctcccagtatcagagattactctgcattaatccacccttgtttgaaatccatcaagtcttgttgagtccaacccaacatcatgtagcacttatcggaagtaaaggacttatggcattagaattacctcagaggtgggggaaggactctgaatttgaaggtggaaaagcaactgtgaattgtagcaccatcccgattgctgagagatttttcaccagctctacctctctgactctgaagcatgctgcatggtatccaagtgagatgctggatccccacatagtgctgttgacatcagacaacgtgataagaatttattctctccgtgagccccagacacccactaaggtgattgtactttcagaagcagaagaggaaagtttaatactcaataaaggaagagcatatacagcgtctctaggagagactgcagtggcgtttgacttcgggcccctggtaaccgtctcaaagaatatatttgaacagaaagacagagatgtggtggcgtatccactgtacatcctgtatgagaatggggagaccttccttacctacgtgagcctgttacacagcccagggaatattgggaagctgttgggcccactgcctatgcatcctgcagctgaagataactacggttacgatgcctgtgctatactctgtttgccctgtgttccaaatatcttagtaattgcgactgagtcaggaatgctgtaccactgtgttgtactagagggagaagaagaagatgaccaaacgttagaaaagtcctgggatcccagggctgacttcatcccttctctgtacgtgtttgagtgtgttgagttagagcttgccctgaagctggcatctggagaggacgatccctttgcctctgacttttcctgcccaattaaactgcacagagatcccaagtgtccttcgagataccactgcagccatgaagctggcgtgcacagtgtggggctgacttggattcacaaactgcacaaatttcttggatcggatgaagaagataaggatagtttacaagaactcactgctgagcagaaatgctttgtggagcacattctttgtacaaagccattgccgtgcaggcagccagctccaattcgaggattctggatcgtcccagacatcctggggcccacaatgatctgcatcaccagtacctatgaatgtctcatacggcctttattaagtacagtccacccagcatctcctcccctgctctgtacccaagaagatgctgaagttgcagagtctccactgcgcattctggctgaaactccagactcctttgagaagcatattaaaagaatcttgcagcgtagtgctgccaacccagcatttctcaaatcatctgaaaaggatttggctccccctcccgaggagtgtcttcagcttatcagcagagccacccaggtgttccgagaacagtacattctcaagcaggacctggccaaggaggagattcagcggagggtcaaattattatgtgaccaaaagaggaaacaactcgaagatctcaattactgtcgagaggaaaggaaaagtctccgggaaatggctgagcgcttagctgacaaatatgaggaagccaaagaaaaacaagaagatatcatgaacaggatgaaaaaagtgcttcacagttttcatgctcagctcccagttctctctgacagtgagagagacatgaagaaagagttacagctgatacctgatcaactgcgacatctaggcaacgccatcaaacaggttactatgaaaaaagattatcaacagcggaagatggaaaaagtgctgagtcctcagaaacccaccattactctcagtgcctaccagcgaaagtgcattcagtccatcctgaaagaagagggtgaacacataagggaaatggtgaagcaaatcaatgacatccgaaatcatgtcaccttctga

SEQ ID NO: 19 - DNA, 1080 bp, Unknown Organism
  Description of Unknown Organism KiflC nucleic acid sequence
gttaaggatgagagatgtgtgtctgattgtctactggacaacttcttctgggtaaacccagaactttctctcaagattcacctccctcagcccgggtggtgtttacttctcaactgtcactgcttcatctatcctcggattcccctaaatctgtcactgtgaggggatagaaactagatacaattccaagaaagcccggcggcctgaagtacagacagactgcgccgagactacaacttccagaatcctttgcgcgcggctaagggcggtgatttgggcgctgtttcccgctaagggaggagatggcagagtcctcggggtctccgcaccgcttgttgtacaagcaggtgggctcgccccactggaaagaaactttcaggcagggatgtctggagagaatgagaaacagcaggcacaggctcctgaacaaatatcgccaggctgcaggtagcacgccggggacagcctcagacagacttcttgtgcaagaagtaatggaggaagagtgggcttctttgcagtctgtggagaattgtccggaggccttgcttcagttggaattgccactggacctagctgtgctgcaggacatcgagcaggagctgtgtaatgaagaaaagtccatcataagtgagtacgaagaggacttagagtttgatgaaagttgtctcaggagaatgttggctgagtgggaagcaaactccctcatctgtcctgtgtgtataaagtacaacctgagaataatgaacagtgtggtcacgtgtccatgtggcctgcacatccctgttcactcaacagacctgacagagcagaagcttcgagcctgtttggaagaaaatgtgaatgagcacagtgtacactgtccccacacccctgtgttytcagtcaccggtggaacagaagagaagcccagtcttctgatgaactgcctgcacttgtgacaccwcacctgggctgtgatcctctagccatcccgacctcacacttcactactgagctgagaacaactcatttcctgagagggccctgtatgcacaagccttttgtatataacggattttatattaaaacttcagacat

SEQ ID NO: 20 - DNA, 840 bp, Unknown Organism
  Description of Unknown Organism KiflC nucleic acid sequence
atgctccctctgctgcgttgcgtgccccgcgccctcggcgccgccgcctcgggcctccgaaccgccatcccggcccagccgcttcggcatctcctgcagcccgcgccccggccatgcctccggcccttcggtttgctcagcgtacgggccggctcggctcggcgctctggcctcctgcagcccccggttccctgcgcgtgcggctgtggcgctctgcacacggaaggagacaaggccttcgttgaattcttgactgatgaaattaaggaagaaaagaagatccagaaacacaagtcccttcccaagatgtctggagattgggagctggaggtgaacggcacggaggctaaattattgcgcaaagttgccggagaaaagatcacggtcactttcaacatcaacaacagcatccctccaacatttgatggtgaggaggagccctcacaggggcagaaggctgaagaacaggagccagaactgacatcaactcccaactttgtggttgaagttacaaagactgatggcaagaagacccttgtactggactgtcactatcctgaggatgagattggacacgaagatgaggccgagagtgatattttctctatcaaggaagttagctttcaggccactggtgactctgagtggagggatacaaactatacactcaacacagattccctggactgggccttgtatgaccacctaatggatttccttgcggaccgaggggtggataacacttttgcggatgagttggtggagctcagcacagccctggagcaccaggaatatatcacctttcttgaggacctcaaaagctttgtcaagaaccagtag

SEQ ID NO: 21 - DNA, 1161 bp, Unknown Organism
  Description of Unknown Organism KiflC nucleic acid sequence
atgctcgtccttcctctgtacgcctccctgccctattcgcagcagcttcgagtctttcaaggggccccaaagggctatcgcaaagtgatcatttcaaccaacatcgctgaaacctccataaccattacaggaataaaatatgtagttgacacgggcatggttaaagcaaagaagtataaccctgacagtggtcttgaggtactagctgtacagagagtatcaaagacccaggcctggcagcgcacaggcagggctgggcgagaggacagtggcatctgctaccgactctacactgaggacgagtttgagaagttcgagaagatgacggtgccagagatccagaggtgtaacctggccagtgtaatacttcagctcctagcaatgaaagtcccaaatgtgctcacctttgacttcatgtccaaaccttctccagatcacattgaggcggccattgcccagctggacctgttaggtgctcttgaacataaggatgaccagcttaccctgactccaattggaagaaagatggcagcattccctttagagcccagatttgccaaaactatcctcctgtcctccaaattccactgtaccgaagagattctgaccatagtctccctgctatctgtggacagtgtcctttacaaccctcctgcccggagagatgaggtgcagagtgtccggaagaaattcatatccagtgagggggatcacatcaccctgctcaatatctatcggaccttcaaaaacatcggtgggaataaggactggtgcaaagagaattttgtcaacagcaagaatatgctgctagtagctgaagtcagagcacagctgagagaaatctgcttaaagatgtcaatgccaatcatgtcatctcgaggggacatggagagtgtccgccgttgcatggctcacagcctctttatgaatactgctgaactccagacagatggcacctatgccaccacggacacacatcagccagtggccatccacccatcatctgtcctcttccactgcaagcctgcctgcgtggtctacacttcactgctctacaccaacaaatgctacatgcgtgacctctgtgtggtggatgccgagtggctctacgaggctgcccctgactacttcaggaggaagctgagaacggccagaaactga

SEQ ID NO: 22 - DNA, 1063 bp, Unknown Organism
  Description of Unknown Organism KiflC nucleic acid sequence
ggaagawggcgtaccagagcctccggctggagtacctgcagatcccgccggtcagccgcgcctatactaccgcctgcgtcctcaccaccgcggccgtgcagttggaattgatcacaccttttcagttatacttcaatcctgaattaatttttaaacattttcaaatctggaggctaatcaccaatttcttattttttggtccagttggattcaattttttgtttaacatgatttttctataccgttattgtcgaatgctagaagaaggctctttccgaggtcggacagcagactttgtatttatgttcctttttggtggatttttaatgactctctttggtttgtttgtgagcttagtttttttaggccaggcctttacaataatgctggtctacgtgtggagccgaaggaacccgtatgtccgcatgaactttttggtcttctaaacttccaggcccccttcctgccctgggtgctcatggggttttccctgttgctggggaactcaattatagtggaccttttgggtattgcagttgggcacatatattttttcttggaagatatatttcccaatcagcctggtggaataagaattctgaaaacaccatctattttgaggactatttttgatacgccagatgaggatcccaattacaacccactacctgaagagcggccgggaggcttcgcctggggtgagggccagcgccttggtgggtaaagcagcggtgccaattacgagacccatctgagaaagactcagtgacatgtccatgggggtcttttatcccttgttgcaaaagcgtggacagttttgacagcttgacagatttttaactccagaagcactttacgggatggtacactgactaacatagaagacatttccaggagtttgccaggggttcctcactatgctggtactaaaagtataacctcttggagccaaaaactggagacccaggcaccctgcctgtgccgtgagcaagtccatgggtacttgagctcagtggcacaggcgggtgctcctcctcctctttgatagacaaggccgtggtgt

SEQ ID NO: 23 - DNA, 654 bp, Unknown Organism
  Description of Unknown Organism KiflC nucleic acid sequence
atgtctgtggatccgatggcctatgaggcccagttctttggcttcacaccacagacttgcctgctgaggatctacgtagcatttcaagaccacctgtttgaagtgatgcaggctgttgaacaggttatcctaaagaagctggaggacattccaaactgtgagatcacccctgtccagactcggaaatgcacagagaagtttctttgcttcatgaaaggacgcttcgataacctttttggcaaaatggagcagctgattttgcagtcgattttgtgtattcctccaaacatcctgcttcctgaagacaagtgtcaggagacgaatcctttcagtgaagaaaaactcgagcttctccaacaggaaatcaaagagttacaggagaaatacaaggttgagttgtgcactgagcaggcccttcttgcagaattagaggagcagaaaactgttaaggccaaactcagagagaccttgactttctttgatgagcttgaaaacatcggcagatatcaaggaactagtaactttagggagagtttggcatccctggtccagagctgcagaaaacttcagagcattagagacaatgtagaaaaagaaagcaggagactggaaacacagtgatttctcagtaatgaaaagagagcatggaaatag